(19) United States
(12) Patent Application Publication
(10) Pub. No.: US 2024/0239931 A1
(43) Pub. Date: Jul. 18, 2024

(54) FLUORINATED RESORCINOL AND HYDROQUINONE ANALOGS AS CURING AGENTS FOR FLUOROELASTOMERS

(71) Applicant: The Chemours Company FC, LLC, Wilmington, DE (US)

(72) Inventors: Alexander Borisovich Shtarov, Wilmington, DE (US); Peter A. Morken, Wilmington, DE (US); Andrii Matsnev, The Plains, OH (US); Seung Kyung Ko, Keongki-do (KR)

(21) Appl. No.: 18/602,643

(22) Filed: Mar. 12, 2024

Related U.S. Application Data
(63) Continuation-in-part of application No. PCT/US23/33096, filed on Sep. 19, 2023.
(60) Provisional application No. 63/408,349, filed on Sep. 20, 2022.

(51) Int. Cl. C08F 214/28

(52) U.S. Cl. CPC C08F 214/28

(57) ABSTRACT

Compositions include a curing agent of a fluorinated resorcinol or hydroquinone of Formula 1:

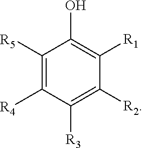

R₁ and R₅ are independently H, halogen, optionally partially or fully halogenated C₁₋₁₈ alkyl, optionally partially or fully halogenated C₁₋₁₈ alkoxy, or X. R₂-R₄ are independently OH, H, halogen, optionally partially or fully halogenated C₁₋₁₈ alkyl, optionally partially or fully halogenated C₁₋₁₈ alkoxy, or X. At least one of R₂, R₃, and R₄ is OH. Not more than 3 of R₁-R₅ are halogen. X is Formula 2 or Formula 3 as disclosed herein. Curable fluoroelastomer compositions, fluoroelastomer masterbatches, curing agent and curing accelerator mixtures, salts, and methods of curing a polyhydroxy-curable fluoroelastomer including a curing agent of a fluorinated resorcinol or hydroquinone of Formula 1 are also disclosed herein.

### REFERENCE TO RELATED APPLICATIONS

This application is a continuation-in-part of International Application No. PCT/US23/33096, filed Sep. 19, 2023, which claims priority to and the benefit of U.S. Provisional Application No. 63/408,349 filed Sep. 20, 2022, both of which are hereby incorporated by reference in their entirety.

### FIELD

This disclosure relates to the curing agents for fluoroelastomers and more particularly to fluorinated resorcinol and hydroquinone analogs useful as curing agents for fluoroelastomers.

### BACKGROUND

Fluoroelastomers have excellent heat resistance, oil resistance, and chemical resistance and have been widely used for sealing materials, containers, and hoses. Examples of fluoroelastomers include copolymers including monomer units of vinylidene fluoride (VF2) and monomer units of at least one other copolymerizable fluorine-containing monomer such as hexafluoropropylene (HFP), tetrafluoroethylene (TFE), chlorotrifluoroethylene (CTFE), vinyl fluoride (VF), or a fluorovinyl ether such as a perfluoro(alkyl vinyl ether) (PAVE). Specific examples of PAVE include perfluoro(methyl vinyl ether), perfluoro(ethyl vinyl ether), and perfluoro(propyl vinyl ether). Other fluoroelastomers include copolymers of TFE with a hydrocarbon olefin, such as ethylene or propylene. Perfluoroelastomers that are copolymers of TFE and PAVE are known.

In order to fully develop the physical properties of elastomers for use in molded elastomeric articles, the polymers must be cured, i.e., vulcanized or crosslinked. This is generally accomplished in connection with the molding process by mixing uncured polymer with a polyfunctional curing agent, heating and molding the mixture into the shape of the desired article, and then further heating the resultant molded mixture, thereby promoting a cross-linking reaction of the curing agent with the polymer to produce a cured fluoroelastomer article.

Certain grades of fluoroelastomers, for example certain copolymers of VF2/HFP or VF2/HFP/TFE that do not include a cure site monomer, are curable using a polyhydroxy compound as a curing agent. 2,2-bis(4-hydroxyphenyl)hexafluoropropane, often referred to as bisphenol AF (BPAF), is a widely used curing agent for polyhydroxy-curable grades of fluoroelastomers. As a curing agent for polyhydroxy-curable grades of fluoroelastomers, BPAF provides good processing of the fluoroelastomer during molding into articles and imparts good properties to cured fluoroelastomer articles. With regard to processing during molding, BPAF does not cause excessive “scorch”, i.e., does not cure too quickly while the article is being molded, but provides desirable short curing times once curing of the article being molded begins. In fluoroelastomer articles after curing, BPAF imparts desirable low “compression set” properties to the fluoroelastomer. Compression set is a commonly used measurement of the ability of an elastomer to return to nearly its original thickness after being compressed at an elevated temperature.

There is currently a proposed restriction in the European Union that includes BPAF in a class of compounds with endocrine disrupting properties. Thus, the use of curing agents other than BPAF is desirable. In U.S. Pat. No. 6,610,790, a number of compounds are listed in addition to BPAF as curing agents for fluoroelastomers, including resorcinol, hydroquinone, and certain alkyl substituted resorcinols and hydroquinones. However, the other curing agents listed in In U.S. Pat. No. 6,610,790 do not provide the good processing and compression set properties that can be provided by BPAF.

### SUMMARY

Fluorinated resorcinol and hydroquinone analogs disclosed herein provide a good balance of processability and compression set properties as alternative curing agents to BPAF in the curing of fluoroelastomers.

In one embodiment, a curable fluoroelastomer composition includes a polyhydroxy-curable fluoroelastomer, a curing agent of Formula 1, and an acid acceptor.

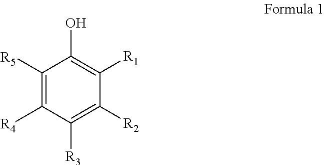

R₁ and R₅ are independently selected from the group consisting of H, halogen, C₁₋₁₈ alkyl which may be partially or fully halogenated, C₁₋₁₈ alkoxy which may be partially or fully halogenated, and X, and R₂, R₃, and R₄ are independently selected from the group consisting of OH, H, halogen, C₁₋₁₈ alkyl which may be partially or fully halogenated, C₁₋₁₈ alkoxy which may be partially or fully halogenated, and X, with the proviso that at least one of R₂, R₃, and R₄ is OH, and with the proviso that not more than 3 of R₁, R₂, R₃, R₄, and R₅ are halogen.

X is selected from the group consisting of Formula 2 and Formula 3:

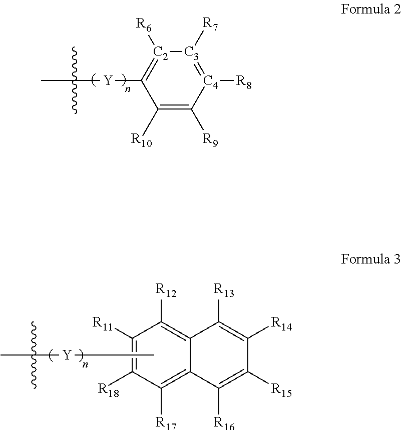

R₆, R₇, R₈, R₉, and R₁₀ are independently selected from the group consisting of H, C₁₋₁₈ alkyl which may be partially or fully halogenated, C₁₋₁₈ alkoxy which may be partially or fully halogenated, partially or fully fluorinated phenyl, acetyl or methylsulfonyl which may be alkyl- or aryl-substituted or partially or fully halogenated, nitro, nitrile, and halogen, with the proviso that N may be substituted at exactly one of C₂, C₃, or C₄ in which case respective R₆, R₇, or R₈ is absent; R₁₁, R₁₂, R₁₃, R₁₄, R₁₅, R₁₆, R₁₇, and R₁₈ are independently selected from the group consisting of H, C₁₋₁₈ alkyl which may be partially or fully halogenated, C₁₋₁₈ alkoxy which may be partially or fully halogenated, nitro, nitrile, and halogen, with the proviso that one of R₁₁, R₁₂, R₁₃, R₁₄, R₁₅, R₁₆, R₁₇, and R₁₈ is a single bond to —(Y)ₙ—; Y is selected from the group consisting of —SO₂—, —C(O)—, —C(CF₃)₂—, and —O— and n is 0 or 1, with the proviso that at least one of R₁, R₂, R₃, R₄, and R₅, is fluorine, a fluorine-containing C₁₋₁₈ alkyl, a fluorine-containing C₁₋₁₈ alkoxy, or X, and with the proviso that, when X is present and none of R₁, R₂, R₃, R₄, and R₅ is fluorine, a fluorine-containing C₁₋₁₈ alkyl, or a fluorine-containing C₁₋₁₈ alkoxy, at least one of R₆, R₇, R₈, R₉, R₁₀, R₁₁, R₁₂, R₁₃, R₁₄, R₁₅, R₁₆, R₁₇, and R₁₈ is fluorine, a fluorine-containing C₁₋₁₈ alkyl, or a fluorine-containing C₁₋₁₈ alkoxy.

In one embodiment of the composition, at least one of R₁ and R₅ is H.

In another embodiment of the composition, at least one of R₁, R₂, R₃, R₄, and R₅ is H when it is an adjacent substituent to OH.

In another embodiment of the composition, no more than one of R₁, R₂, R₃, R₄, and R₅ is X.

In another embodiment of the composition, no more than 2 of R₁, R₂, R₃, R₄, and R₅ are halogen.

In another embodiment of the composition, only one of R₂, R₃, and R₄ is OH.

In another embodiment of the composition, either R₂ or R₄ is OH.

In another embodiment of the composition, R₁ and R₅ are selected from the group consisting of H, fluorine, C₁₋₁₈ alkyl which may be partially or fully fluorinated, C₁₋₁₈ alkoxy which may be partially or fully fluorinated, and X; and R₂, R₃, and R₄ are independently selected from the group consisting of OH, H, fluorine, C₁₋₁₈ alkyl which may be partially or fully fluorinated, C₁₋₁₈ alkoxy which may be partially or fully fluorinated, and X.

In another embodiment of the composition, one of R₁, R₂, R₃, R₄, and R₅, is X and X is Formula 2.

In another embodiment of the composition, R₆, R₇, R₈, R₉, and R₁₀ are independently selected from the group consisting of H, fluorine, C₁₋₆ alkyl which may be partially or fully fluorinated, and C₁₋₆ alkoxy which may be partially or fully fluorinated and at least one of R₆, R₇, R₈, R₉, and R₁₀ is fluorine, C₁₋₆ alkyl which may be partially or fully fluorinated, or C₁₋₆ alkoxy which may be partially or fully fluorinated.

In another embodiment of the composition, R₆, R₇, R₈, R₉, and R₁₀ are independently selected from the group consisting of H, fluorine, perfluoromethyl, and perfluoromethoxy and at least one of R₆, R₇, R₈, R₉, and R₁₀ is fluorine, perfluoromethyl, or perfluoromethoxy.

In another embodiment of the composition, n is 0.

In another embodiment of the composition, —Y— is —O—.

In another embodiment of the composition, the curing agent is selected from the group consisting of:

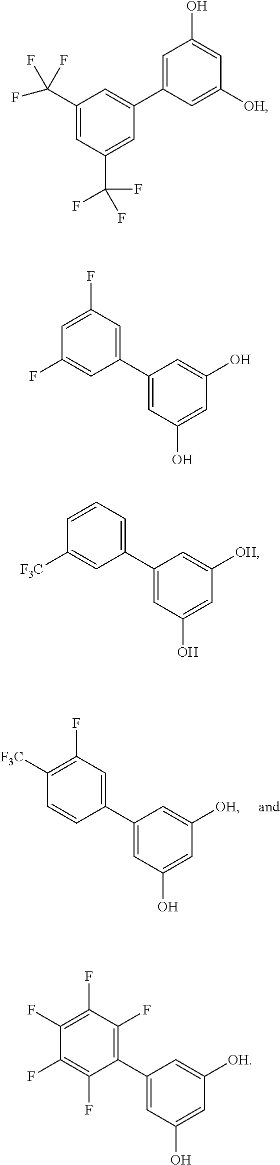 [SMILES: Oc1cc(O)cc(-c2cc(C(F)(F)F)cc(C(F)(F)F)c2)c1 ; Oc1cc(O)cc(-c2cc(F)cc(F)c2)c1 ; Oc1cc(O)cc(-c2cccc(C(F)(F)F)c2)c1 ; Oc1cc(O)cc(-c2ccc(C(F)(F)F)c(F)c2)c1 ; "and" ; Oc1cc(O)cc(-c2c(F)c(F)c(F)c(F)c2F)c1]

In another embodiment of the composition, the composition contains about 0.1 to about 10 parts by weight of said curing agent per 100 parts by weight of fluoroelastomer.

In another embodiment of the composition, the polyhydroxy-curable fluoroelastomer is a copolymer of hexafluoropropylene and vinylidene fluoride.

In another embodiment of the composition, the polyhydroxy-curable fluoroelastomer is a terpolymer of hexafluoropropylene, vinylidene fluoride, and tetrafluoroethylene.

In another embodiment of the composition, the acid acceptor is selected from the group consisting of powdered magnesium oxide, calcium hydroxide, and a combination thereof.

In another embodiment of the composition, the curable fluoroelastomer composition is free of 2,2-bis(4 hydroxyphenyl)hexafluoropropane.

In another embodiment, a fluoroelastomer masterbatch includes a polyhydroxy-curable fluoropolymer and curing agent of Formula 1. The curing agent is present at a concentration of about 1 wt % to about 50 wt %.

In one embodiment of the fluoroelastomer masterbatch, the concentration of said curing agent is about 20 wt % to about 40 wt %.

In another embodiment, a curing agent and curing accelerator mixture includes a curing agent of Formula 1 and a curing accelerator selected from the group consisting of a quaternary phosphonium salt, a quaternary ammonium salt, and a tertiary sulfonium salt.

In one embodiment of the curing agent and curing accelerator mixture, the curing accelerator is a tertiary sulfonium salt.

In another embodiment of the curing agent and curing accelerator mixture, the curing accelerator is a quaternary ammonium salt.

In another embodiment of the curing agent and curing accelerator mixture, the quaternary ammonium salt is tetrabutylammonium hydrogen sulfate.

In another embodiment of the curing agent and curing accelerator mixture, the curing accelerator is a quaternary phosphonium salt.

In another embodiment of the curing agent and curing accelerator mixture, the quaternary phosphonium salt is benzyl triphenyl phosphonium chloride.

In yet another embodiment, a salt for use as a fluoroelastomer curing agent and curing accelerator includes a quaternary phosphonium salt or quaternary ammonium salt derived from a compound of Formula 1.

In one embodiment of the salt, the curing accelerator is a quaternary ammonium salt.

In another embodiment of the salt, the quaternary ammonium salt is tetrabutylammonium hydrogen sulfate.

In another embodiment of the salt, the curing accelerator is a quaternary phosphonium salt.

In another embodiment of the salt, the salt is a benzyl triphenyl phosphonium salt.

In another embodiment, a method of curing a polyhydroxy-curable fluoroelastomer includes forming a curable fluoroelastomer composition including a polyhydroxy-curable fluoroelastomer, a curing agent of Formula 1, and an acid acceptor and heating the curable fluoroelastomer composition to cure the polyhydroxy-curable fluoroelastomer.

In one embodiment of the method, the curable fluoroelastomer composition is free of 2,2-bis(4 hydroxyphenyl)hexafluoropropane.

In another embodiment, an article is cured by the method.

In one embodiment of the article, the article is free of or substantially free of 2,2-bis(4 hydroxyphenyl)hexafluoropropane.

In another embodiment, a compound is of Formula 1A.

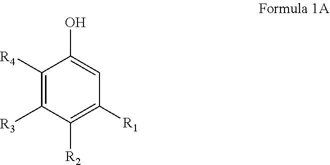

One of R₁ and R₂ is H and the other is OH. One of R₃ and R₄ is H and the other is selected from the group consisting of Formula 2A and Formula 3A.

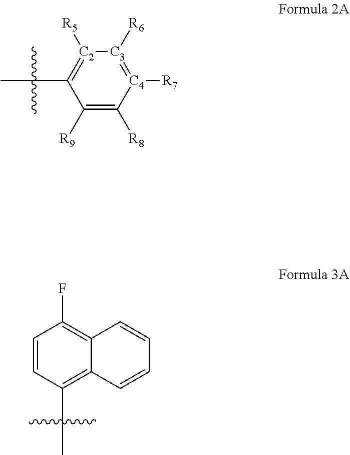

R₅, R₆, R₇, R₈, and R₉ are independently selected from the group consisting of H, F, CF₃, partially or fully fluorinated phenyl, OCF₃, CH₃, nitro, and nitrile; with the proviso that at least one of R₅, R₆, R₇, R₈, and R₉ is selected from the group consisting of F, CF₃, partially or fully fluorinated phenyl, and OCF₃; with the proviso that when R₅, R₆, R₇, R₈, and R₉ are independently selected from the group consisting of H and F, at least two are H and at least two are F; with the proviso that when R₁ is OH, R₃ is Formula 2A, and R₅, R₆, R₇, R₈, and R₉ are independently selected from the group consisting of H and CF₃, at least one of R₆ and R₈ is H; and with the proviso that N may be substituted at exactly one of C₂, C₃, or C₄ in which case respective R₅, R₆, or R₇ is absent.

In one embodiment of the compound, R₁ is OH and R₂ is H.

In some embodiments, the ring in Formula 2A is a benzyl ring.

In some embodiments, N is substituted at exactly one of C₂, C₃, or C₄ and the ring in Formula 2A is a pyridinyl ring.

Other features and advantages of the present invention will be apparent from the following more detailed description of the preferred embodiments, taken in conjunction with the accompanying drawings which illustrate, by way of example, the principles of the invention.

### DETAILED DESCRIPTION

Provided are exemplary fluorinated resorcinol and hydroquinone analogs that provide a good balance of processability, and compression set properties as curing agents to replace BPAF in the curing of fluoroelastomers.

In exemplary embodiments, the curing agent provides cure properties and cured fluoroelastomer properties similar to BPAF as a curing agent. Such cure properties may be measured by a moving die rheometer (MDR) and may include, but are not limited to, the minimum S′ torque (M_{L}), the maximum S′ torque achieved during a specified time period (M_{H}), the (scorch) time to increase one unit of S′ torque from M_{L} (ts1), the (scorch) time to increase two units of S′ torque from M_{L} (ts2), the (cure) time to an increase of 50% of S′ torque from M_{L} to M_{H} (t₅₀), and/or the (cure) time to an increase of 90% of S′ torque from M_{L} to M_{H} (t₉₀). Such cured fluoroelastomer properties may include, but are not limited to, compression set resistance, tensile strength (TS), the elongation at break (EB), and the elastic modulus at 100% (M100), and/or fluid aged properties.

In exemplary embodiments, the curing agent is of Formula 1:

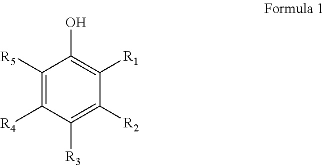

where R₁ and R₅ are independently selected from the group consisting of H, halogen, C₁₋₁₈ alkyl which may be partially or fully halogenated, C₁₋₁₈ alkoxy which may be partially or fully halogenated, and X, and R₂, R₃, and R₄ are independently selected from the group consisting of OH, H, halogen, C₁₋₁₈ alkyl which may be partially or fully halogenated, C₁₋₁₈ alkoxy which may be partially or fully halogenated, and X, with the proviso that at least one of R₂, R₃, and R₄ is OH and with the proviso that not more than 3 of R₁, R₂, R₃, R₄, and R₅ are halogen;
where X is selected from the group consisting of Formula 2 and Formula 3:

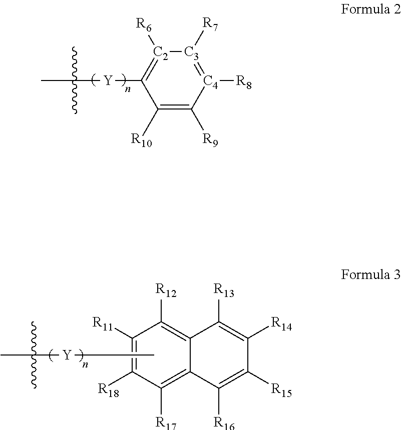

where R₆, R₇, R₈, R₉, and R₁₀ are independently selected from the group consisting of H, C₁₋₁₈ alkyl which may be partially or fully halogenated, C₁₋₁₈ alkoxy which may be partially or fully halogenated, partially or fully fluorinated phenyl, acetyl or methylsulfonyl which may be alkyl- or aryl-substituted or partially or fully halogenated, nitro, nitrile, and halogen, with the proviso that N may be substituted at exactly one of C₂, C₃, or C₄ in which case respective R₆, R₇, or R₈ is absent such that the ring in Formula 2 is a pyridinyl ring; and R₁₁, R₁₂, R₁₃, R₁₄, R₁₅, R₁₆, R₁₇, and R₁₈ are independently selected from the group consisting of H, C₁₋₁₈ alkyl which may be partially or fully halogenated, C₁₋₁₈ alkoxy which may be partially or fully halogenated, nitro, nitrile, and halogen, with the proviso that one of R₁₁, R₁₂, R₁₃, R₁₄, R₁₅, R₁₆, R₁₇, and R₁₈ is a single bond to —(Y)ₙ—; Y is selected from the group consisting of —SO₂—, —C(O)—, —C(CF₃)₂—, and —O— and n is 0 or 1, with the proviso that at least one of R₁, R₂, R₃, R₄, and R₅, is fluorine, a fluorine-containing C₁₋₁₈ alkyl, a fluorine-containing C₁₋₁₈ alkoxy, or X, and with the proviso that, when X is present and none of R₁, R₂, R₃, R₄, and R₅ is fluorine, a fluorine-containing C₁₋₁₈ alkyl, or a fluorine-containing C₁₋₁₈ alkoxy, at least one of R₆, R₇, R₈, R₉, R₁₀, R₁₁, R₁₂, R₁₃, R₁₄, R₁₅, R₁₆, R₁₇, and R₁₈ is fluorine, a fluorine-containing C₁₋₁₈ alkyl, partially or fully fluorinated phenyl, or a fluorine-containing C₁₋₁₈ alkoxy.

In some embodiments, the ring in Formula 2 is a benzyl ring.

In some embodiments, N is substituted at exactly one of C₂, C₃, or C₄ and the ring in Formula 2 is a pyridinyl ring.

In some embodiments, at least one of R₁ and R₅ is H.

In some embodiments, at least one of R₁, R₂, R₃, R₄, and R₅ is H when it is an adjacent substituent to OH.

In some embodiments, no more than one of R₁, R₂, R₃, R₄, and R₅ is X.

In some embodiments, no more than 2 of R₁, R₂, R₃, R₄, and R₅ are halogen.

In some embodiments, only one of R₂, R₃, and R₄ is OH.

In some embodiments, either R₂ or R₄ is OH.

In some embodiments, R₁ and R₅ are selected from the group consisting of H, fluorine, C₁₋₁₈ alkyl which may be partially or fully fluorinated, C₁₋₁₈ alkoxy which may be partially or fully fluorinated, and X; and R₂, R₃, and R₄ are independently selected from the group consisting of OH, H, fluorine, C₁₋₁₈ alkyl which may be partially or fully fluorinated, C₁₋₁₈ alkoxy which may be partially or fully fluorinated, and X.

In some embodiments, one of R₁, R₂, R₃, R₄, and R₅, is X and X is Formula 2.

In some embodiments, R₆, R₇, R₈, R₉, and R₁₀ are independently selected from the group consisting of H, fluorine, C₁₋₆ alkyl which may be partially or fully fluorinated, and C₁₋₆ alkoxy which may be partially or fully fluorinated and at least one of R₆, R₇, R₈, R₉, and R₁₀ is fluorine, C₁₋₆ alkyl which may be partially or fully fluorinated, or C₁₋₆ alkoxy which may be partially or fully fluorinated.

In some embodiments, R₆, R₇, R₈, R₉, and R₁₀ are independently selected from the group consisting of H, fluorine, perfluoromethyl, and perfluoromethoxy and at least one of R₆, R₇, R₈, R₉, and R₁₀ is fluorine, perfluoromethyl, or perfluoromethoxy.

In some embodiments, n is 0.

In some embodiments, —Y— is —O—.

In some embodiments, the curing agent is selected from the 19 specific single-ring resorcinol or hydroquinone analogs of Formula 1 shown in Table 1.

TABLE 1
Single-Ring Curing Agents

| No. | R1 | R2 | R3 | R4 | R5 |
|---|---|---|---|---|---|
| 1 | H | OH | H | OCF2CF2H | H |
| 2 | H | OH | OCF2CF2H | H | H |
| 3 | H | H | OH | OCF2CF2H | H |
| 4 | H | OH | H | OCF2CFHCF3 | H |
| 5 | H | OH | OCF2CFHCF3 | H | H |
| 6 | H | H | OH | OCF2CFHCF3 | H |
| 7 | H | OH | H | OCF2CFHOCF3 | H |
| 8 | H | OH | OCF2CFHOCF3 | H | H |
| 9 | H | H | OH | OCF2CFHOCF3 | H |
| 10 | H | OH | H | OCF3 | H |
| 11 | H | OH | OCF3 | H | H |
| 12 | H | OH | H | CF(CF3)2 | H |
| 13 | H | OH | CF(CF3)2 | H | H |
| 14 | H | OH | H | OCF2H | H |
| 15 | H | OH | OCF2H | H | H |
| 16 | H | H | OH | OCF2H | H |
| 17 | CF3 | OH | H | H | H |
| 18 | CF3 | OH | H | CF3 | H |
| 19 | CF3 | H | OH | CF3 | H |

In some embodiments, the curing agent is selected from the 26 specific resorcinol or hydroquinone analog salts of Formula 1 shown in Table 2, where the cation is benzyl triphenyl phosphonium (BTPP+).

TABLE 2
Single-Ring Salt Curing Agents

| | | Anion | | | | |
|---|---|---|---|---|---|---|
| No. | Cation | R1 | R2 | R3 | R4 | R5 |
| 1 | BTPP+ | H | O(—) | H | OCF2CF2H | H |
| 2 | BTPP+ | H | O(—) | OCF2CF2H | H | H |
| 3 | BTPP+ | H | H | O(—) | OCF2CF2H | H |
| 4 | BTPP+ | H | O(—) | H | OCF2CFHCF3 | H |
| 5 | BTPP+ | H | O(—) | OCF2CFHCF3 | H | H |
| 6 | BTPP+ | H | H | O(—) | OCF2CFHCF3 | H |
| 7 | BTPP+ | H | O(—) | H | OCF2CFHOCF3 | H |
| 8 | BTPP+ | H | O(—) | OCF2CFHOCF3 | H | H |
| 9 | BTPP+ | H | H | O(—) | OCF2CFHOCF3 | H |
| 10 | BTPP+ | H | O(—) | H | OCF3 | H |
| 11 | BTPP+ | H | O(—) | OCF3 | H | H |
| 12 | BTPP+ | H | O(—) | H | CF(CF3)2 | H |
| 13 | BTPP+ | H | O(—) | CF(CF3)2 | H | H |
| 14 | BTPP+ | H | O(—) | H | OCF2H | H |
| 15 | BTPP+ | H | O(—) | OCF2H | H | H |
| 16 | BTPP+ | H | H | O(—) | OCF2H | H |
| 17 | BTPP+ | CF3 | O(—) | H | H | H |
| 18 | BTPP+ | CF3 | O(—) | H | CF3 | H |
| 19 | BTPP+ | CF3 | H | O(—) | CF3 | H |
| 20 | BTPP+ | F | H | O(—) | H | H |
| 21 | BTPP+ | OCF3 | H | O(—) | H | H |
| 22 | BTPP+ | CF(CF3)2 | H | O(—) | H | H |
| 23 | BTPP+ | CF3 | H | O(—) | H | H |
| 24 | BTPP+ | H | O(—) | H | F | H |
| 25 | BTPP+ | F | O(—) | H | H | H |
| 26 | BTPP+ | H | O(—) | F | H | H |

In some embodiments, the curing agent is selected from the 196 specific multi-ring resorcinol analogs of Formula 1 (F-1), including Formula 2 (F-2) or Formula 3 (F-3), shown in Table 3, where the pendant ring(s) of F-1 or F-2 is at R5 of F-1.

TABLE 3
Resorcinol Analog Curing Agents with Pendant R5 Ring(s)

| No. | R1 | R2 | R3 | R4 | R5 | Y | n | R6 | R7 | R8 | R9 | R10 | C2 | C3 | C4 |
|---|---|---|---|---|---|---|---|---|---|---|---|---|---|---|---|
| 1 | H | OH | H | H | F-2 | | 0 | F | H | H | H | H | C | C | C |
| 2 | H | OH | H | H | F-2 | | 0 | H | F | H | H | H | C | C | C |
| 3 | H | OH | H | H | F-2 | | 0 | H | H | F | H | H | C | C | C |
| 4 | H | OH | H | H | F-2 | | 0 | F | F | H | H | H | C | C | C |
| 5 | H | OH | H | H | F-2 | | 0 | F | H | F | H | H | C | C | C |
| 6 | H | OH | H | H | F-2 | | 0 | F | H | H | H | H | C | C | C |
| 7 | H | OH | H | H | F-2 | | 0 | F | H | H | F | F | C | C | C |
| 8 | H | OH | H | H | F-2 | | 0 | F | F | F | H | H | C | C | C |
| 9 | H | OH | H | H | F-2 | | 0 | F | F | H | H | H | C | C | C |
| 10 | H | OH | H | H | F-2 | | 0 | F | F | H | F | F | C | C | C |
| 11 | H | OH | H | H | F-2 | | 0 | F | H | F | H | H | C | C | C |
| 12 | H | OH | H | H | F-2 | | 0 | F | H | F | F | F | C | C | C |
| 13 | H | OH | H | H | F-2 | | 0 | H | F | F | H | H | C | C | C |
| 14 | H | OH | H | H | F-2 | | 0 | F | F | F | F | H | C | C | C |
| 15 | H | OH | H | H | F-2 | | 0 | F | F | F | F | F | C | C | C |
| 16 | H | OH | H | H | F-2 | | 0 | F | F | H | F | F | C | C | C |
| 17 | H | OH | H | H | F-2 | | 0 | F | F | F | F | F | C | C | C |
| 18 | H | OH | H | H | F-2 | | 0 | CF3 | H | H | H | H | C | C | C |
| 19 | H | OH | H | H | F-2 | | 0 | H | CF3 | H | H | H | C | C | C |
| 20 | H | OH | H | H | F-2 | | 0 | F | H | CF3 | H | H | C | C | C |
| 21 | H | OH | H | H | F-2 | | 0 | CF3 | H | CF3 | H | H | C | C | C |
| 22 | H | OH | H | H | F-2 | | 0 | H | CF3 | CF3 | H | H | C | C | C |
| 23 | H | OH | H | H | F-2 | | 0 | CF3 | F | H | H | H | C | C | C |
| 24 | H | OH | H | H | F-2 | | 0 | CF3 | H | F | H | H | C | C | C |
| 25 | H | OH | H | H | F-2 | | 0 | CF3 | H | H | F | H | C | C | C |
| 26 | H | OH | H | H | F-2 | | 0 | CF3 | H | H | H | F | C | C | C |
| 27 | H | OH | H | H | F-2 | | 0 | F | CF3 | H | H | H | C | C | C |
| 28 | H | OH | H | H | F-2 | | 0 | H | CF3 | F | H | H | C | C | C |
| 29 | H | OH | H | H | F-2 | | 0 | H | CF3 | H | F | H | C | C | C |
| 30 | H | OH | H | H | F-2 | | 0 | H | CF3 | H | H | F | C | C | C |
| 31 | H | OH | H | H | F-2 | | 0 | F | H | CF3 | H | H | C | C | C |
| 32 | H | OH | H | H | F-2 | | 0 | H | F | CF3 | H | H | C | C | C |
| 33 | H | OH | H | H | F-2 | | 0 | H | H | CF3 | F | H | C | C | C |
| 34 | H | OH | H | H | F-2 | | 0 | H | H | CF3 | H | F | C | C | C |
| 35 | H | OH | H | H | F-2 | | 0 | CF3 | F | F | H | H | C | C | C |
| 36 | H | OH | H | H | F-2 | | 0 | CF3 | F | H | F | H | C | C | C |
| 37 | H | OH | H | H | F-2 | | 0 | CF3 | F | H | H | F | C | C | C |
| 38 | H | OH | H | H | F-2 | | 0 | CF3 | H | F | F | H | C | C | C |
| 39 | H | OH | H | H | F-2 | | 0 | CF3 | H | F | H | F | C | C | C |
| 40 | H | OH | H | H | F-2 | | 0 | CF3 | H | H | F | F | C | C | C |
| 41 | H | OH | H | H | F-2 | | 0 | F | CF3 | F | H | H | C | C | C |
| 42 | H | OH | H | H | F-2 | | 0 | F | CF3 | H | F | H | C | C | C |
| 43 | H | OH | H | H | F-2 | | 0 | F | CF3 | H | H | F | C | C | C |
| 44 | H | OH | H | H | F-2 | | 0 | H | CF3 | F | F | H | C | C | C |
| 45 | H | OH | H | H | F-2 | | 0 | H | CF3 | F | H | F | C | C | C |
| 46 | H | OH | H | H | F-2 | | 0 | H | CF3 | H | F | F | C | C | C |
| 47 | H | OH | H | H | F-2 | | 0 | F | F | CF3 | H | H | C | C | C |
| 48 | H | OH | H | H | F-2 | | 0 | F | H | CF3 | F | H | C | C | C |
| 49 | H | OH | H | H | F-2 | | 0 | F | H | CF3 | H | F | C | C | C |
| 50 | H | OH | H | H | F-2 | | 0 | H | F | CF3 | F | H | C | C | C |
| 51 | H | OH | H | H | F-2 | | 0 | H | F | CF3 | H | F | C | C | C |
| 52 | H | OH | H | H | F-2 | | 0 | CF3 | F | F | F | H | C | C | C |
| 53 | H | OH | H | H | F-2 | | 0 | CF3 | F | F | H | F | C | C | C |
| 54 | H | OH | H | H | F-2 | | 0 | CF3 | F | H | F | F | C | C | C |
| 55 | H | OH | H | H | F-2 | | 0 | F | CF3 | F | F | H | C | C | C |
| 56 | H | OH | H | H | F-2 | | 0 | F | CF3 | F | H | F | C | C | C |
| 57 | H | OH | H | H | F-2 | | 0 | F | CF3 | H | F | F | C | C | C |
| 58 | H | OH | H | H | F-2 | | 0 | H | CF3 | F | F | F | C | C | C |
| 59 | H | OH | H | H | F-2 | | 0 | F | F | CF3 | F | H | C | C | C |
| 60 | H | OH | H | H | F-2 | | 0 | F | F | CF3 | H | F | C | C | C |
| 61 | H | OH | H | H | F-2 | | 0 | F | F | CF3 | F | F | C | C | C |
| 62 | H | OH | H | H | F-2 | | 0 | H | CF3 | F | CF3 | H | C | C | C |
| 63 | H | OH | H | H | F-2 | | 0 | CF3 | F | CF3 | H | H | C | C | C |
| 64 | H | OH | H | H | F-2 | | 0 | OCF3 | H | H | H | H | C | C | C |
| 65 | H | OH | H | H | F-2 | | 0 | H | OCF3 | H | H | H | C | C | C |
| 66 | H | OH | H | H | F-2 | | 0 | H | H | OCF3 | H | H | C | C | C |
| 67 | H | OH | H | H | F-2 | | 0 | OCF3 | H | OCF3 | H | H | C | C | C |
| 68 | H | OH | H | H | F-2 | | 0 | H | OCF3 | OCF3 | H | H | C | C | C |
| 69 | H | OH | H | H | F-2 | | 0 | H | OCF3 | H | OCF3 | H | C | C | C |
| 70 | H | OH | H | H | F-2 | | 0 | OCF3 | F | H | H | H | C | C | C |
| 71 | H | OH | H | H | F-2 | | 0 | OCF3 | H | F | H | H | C | C | C |
| 72 | H | OH | H | H | F-2 | | 0 | OCF3 | H | H | F | H | C | C | C |
| 73 | H | OH | H | H | F-2 | | 0 | OCF3 | H | H | H | F | C | C | C |
| 74 | H | OH | H | H | F-2 | | 0 | F | OCF3 | H | H | H | C | C | C |
| 75 | H | OH | H | H | F-2 | | 0 | H | OCF3 | F | H | H | C | C | C |
| 76 | H | OH | H | H | F-2 | | 0 | H | OCF3 | H | F | H | C | C | C |
| 77 | H | OH | H | H | F-2 | | 0 | H | OCF3 | H | H | F | C | C | C |
| 78 | H | OH | H | H | F-2 | | 0 | F | H | OCF3 | H | H | C | C | C |
| 79 | H | OH | H | H | F-2 | | 0 | H | F | OCF3 | H | H | C | C | C |
| 80 | H | OH | H | H | F-2 | | 0 | H | H | OCF3 | F | H | C | C | C |
| 81 | H | OH | H | H | F-2 | | 0 | H | H | OCF3 | H | F | C | C | C |
| 82 | H | OH | H | H | F-2 | | 0 | OCF3 | F | F | H | H | C | C | C |
| 83 | H | OH | H | H | F-2 | | 0 | OCF3 | F | H | F | H | C | C | C |
| 84 | H | OH | H | H | F-2 | | 0 | OCF3 | F | H | H | F | C | C | C |
| 85 | H | OH | H | H | F-2 | | 0 | OCF3 | H | F | F | H | C | C | C |
| 86 | H | OH | H | H | F-2 | | 0 | OCF3 | H | F | H | F | C | C | C |
| 87 | H | OH | H | H | F-2 | | 0 | OCF3 | H | H | F | F | C | C | C |
| 88 | H | OH | H | H | F-2 | | 0 | F | OCF3 | F | H | H | C | C | C |
| 89 | H | OH | H | H | F-2 | | 0 | F | OCF3 | H | F | H | C | C | C |
| 90 | H | OH | H | H | F-2 | | 0 | F | OCF3 | H | H | F | C | C | C |
| 91 | H | OH | H | H | F-2 | | 0 | H | OCF3 | F | F | H | C | C | C |
| 92 | H | OH | H | H | F-2 | | 0 | H | OCF3 | F | H | F | C | C | C |
| 93 | H | OH | H | H | F-2 | | 0 | H | OCF3 | H | F | F | C | C | C |
| 94 | H | OH | H | H | F-2 | | 0 | F | F | OCF3 | H | H | C | C | C |
| 95 | H | OH | H | H | F-2 | | 0 | F | H | OCF3 | F | H | C | C | C |
| 96 | H | OH | H | H | F-2 | | 0 | F | H | OCF3 | H | F | C | C | C |
| 97 | H | OH | H | H | F-2 | | 0 | H | F | OCF3 | F | H | C | C | C |
| 98 | H | OH | H | H | F-2 | | 0 | H | F | OCF3 | H | F | C | C | C |
| 99 | H | OH | H | H | F-2 | | 0 | OCF3 | F | F | F | H | C | C | C |
| 100 | H | OH | H | H | F-2 | | 0 | OCF3 | F | F | H | F | C | C | C |
| 101 | H | OH | H | H | F-2 | | 0 | OCF3 | F | H | F | F | C | C | C |
| 102 | H | OH | H | H | F-2 | | 0 | F | OCF3 | F | F | H | C | C | C |
| 103 | H | OH | H | H | F-2 | | 0 | F | OCF3 | F | H | F | C | C | C |
| 104 | H | OH | H | H | F-2 | | 0 | F | OCF3 | H | F | F | C | C | C |
| 105 | H | OH | H | H | F-2 | | 0 | H | OCF3 | F | F | F | C | C | C |
| 106 | H | OH | H | H | F-2 | | 0 | F | F | OCF3 | F | H | C | C | C |
| 107 | H | OH | H | H | F-2 | | 0 | F | F | OCF3 | H | F | C | C | C |
| 108 | H | OH | H | H | F-2 | | 0 | F | F | OCF3 | F | F | C | C | C |
| 109 | H | OH | H | H | F-2 | | 0 | H | OCF3 | F | OCF3 | H | C | C | C |
| 110 | H | OH | H | H | F-2 | | 0 | OCF3 | F | OCF3 | H | H | C | C | C |
| 111 | H | OH | H | H | F-2 | | 0 | CF2H | H | H | H | H | C | C | C |
| 112 | H | OH | H | H | F-2 | | 0 | H | CF2H | H | H | H | C | C | C |
| 113 | H | OH | H | H | F-2 | | 0 | H | H | CF2H | H | H | C | C | C |
| 114 | H | OH | H | H | F-2 | | 0 | CF2H | H | CF2H | H | H | C | C | C |
| 115 | H | OH | H | H | F-2 | | 0 | H | CF2H | CF2H | H | H | C | C | C |
| 116 | H | OH | H | H | F-2 | | 0 | H | CF2H | H | CF2H | H | C | C | C |
| 117 | H | OH | H | H | F-2 | | 0 | CF2H | F | H | H | H | C | C | C |
| 118 | H | OH | H | H | F-2 | | 0 | CF2H | H | F | H | H | C | C | C |
| 119 | H | OH | H | H | F-2 | | 0 | CF2H | H | H | F | H | C | C | C |
| 120 | H | OH | H | H | F-2 | | 0 | CF2H | H | H | H | F | C | C | C |
| 121 | H | OH | H | H | F-2 | | 0 | F | CF2H | H | H | H | C | C | C |
| 122 | H | OH | H | H | F-2 | | 0 | H | CF2H | F | H | H | C | C | C |
| 123 | H | OH | H | H | F-2 | | 0 | H | CF2H | H | F | H | C | C | C |
| 124 | H | OH | H | H | F-2 | | 0 | H | CF2H | H | H | F | C | C | C |
| 125 | H | OH | H | H | F-2 | | 0 | F | H | CF2H | H | H | C | C | C |
| 126 | H | OH | H | H | F-2 | | 0 | H | F | CF2H | H | H | C | C | C |
| 127 | H | OH | H | H | F-2 | | 0 | H | H | CF2H | F | H | C | C | C |
| 128 | H | OH | H | H | F-2 | | 0 | H | H | CF2H | H | F | C | C | C |
| 129 | H | OH | H | H | F-2 | | 0 | CF2H | F | F | H | H | C | C | C |
| 130 | H | OH | H | H | F-2 | | 0 | CF2H | F | H | F | H | C | C | C |
| 131 | H | OH | H | H | F-2 | | 0 | CF2H | F | H | H | F | C | C | C |
| 132 | H | OH | H | H | F-2 | | 0 | CF2H | H | F | F | H | C | C | C |
| 133 | H | OH | H | H | F-2 | | 0 | CF2H | H | F | H | F | C | C | C |
| 134 | H | OH | H | H | F-2 | | 0 | CF2H | H | H | F | F | C | C | C |
| 135 | H | OH | H | H | F-2 | | 0 | F | CF2H | F | H | H | C | C | C |
| 136 | H | OH | H | H | F-2 | | 0 | F | CF2H | H | F | H | C | C | C |
| 137 | H | OH | H | H | F-2 | | 0 | F | CF2H | H | H | F | C | C | C |
| 138 | H | OH | H | H | F-2 | | 0 | H | CF2H | F | F | H | C | C | C |
| 139 | H | OH | H | H | F-2 | | 0 | H | CF2H | F | H | F | C | C | C |
| 140 | H | OH | H | H | F-2 | | 0 | H | CF2H | H | F | F | C | C | C |
| 141 | H | OH | H | H | F-2 | | 0 | F | F | CF2H | H | H | C | C | C |
| 142 | H | OH | H | H | F-2 | | 0 | F | H | CF2H | F | H | C | C | C |
| 143 | H | OH | H | H | F-2 | | 0 | F | H | CF2H | H | F | C | C | C |
| 144 | H | OH | H | H | F-2 | | 0 | H | F | CF2H | F | H | C | C | C |
| 145 | H | OH | H | H | F-2 | | 0 | H | F | CF2H | H | F | C | C | C |
| 146 | H | OH | H | H | F-2 | | 0 | CF2H | F | F | F | H | C | C | C |
| 147 | H | OH | H | H | F-2 | | 0 | CF2H | F | F | H | F | C | C | C |
| 148 | H | OH | H | H | F-2 | | 0 | CF2H | F | H | F | F | C | C | C |
| 149 | H | OH | H | H | F-2 | | 0 | F | CF2H | F | F | H | C | C | C |
| 150 | H | OH | H | H | F-2 | | 0 | F | CF2H | F | H | F | C | C | C |
| 151 | H | OH | H | H | F-2 | | 0 | F | CF2H | H | F | F | C | C | C |
| 152 | H | OH | H | H | F-2 | | 0 | H | CF2H | F | F | F | C | C | C |
| 153 | H | OH | H | H | F-2 | | 0 | F | F | CF2H | F | H | C | C | C |
| 154 | H | OH | H | H | F-2 | | 0 | F | F | CF2H | H | F | C | C | C |
| 155 | H | OH | H | H | F-2 | | 0 | F | F | CF2H | F | F | C | C | C |
| 156 | H | OH | H | H | F-2 | | 0 | H | CF2H | F | CF2H | H | C | C | C |
| 157 | H | OH | H | H | F-2 | | 0 | CF2H | F | CF2H | H | H | C | C | C |
| 158 | H | OH | H | H | F-2 | | 0 | C6F5 | H | H | H | H | C | C | C |
| 159 | H | OH | H | H | F-2 | | 0 | H | C6F5 | H | H | H | C | C | C |
| 160 | H | OH | H | H | F-2 | | 0 | H | H | C6F5 | H | H | C | C | C |
| 163 | H | OH | H | H | F-2 | | 0 | F | F | C6F5 | F | F | C | C | C |
| 162 | H | OH | H | H | F-2 | | 0 | F | C6F5 | F | F | F | C | C | C |
| 163 | H | OH | H | H | F-2 | | 0 | C6F5 | F | F | F | F | C | C | C |
| 164 | H | OH | H | H | F-2 | | 0 | | F | F | F | F | N | C | C |
| 165 | H | OH | H | H | F-2 | | 0 | F | | F | F | F | C | N | C |
| 166 | H | OH | H | H | F-2 | | 0 | F | F | | F | F | C | C | N |
| 167 | H | OH | H | H | F-2 | | 0 | | H | F | F | F | N | C | C |
| 168 | H | OH | H | H | F-2 | | 0 | | F | H | F | F | N | C | C |
| 169 | H | OH | H | H | F-2 | | 0 | | F | F | H | F | N | C | C |
| 170 | H | OH | H | H | F-2 | | 0 | | F | F | F | H | N | C | C |
| 171 | H | OH | H | H | F-2 | | 0 | H | | F | F | F | C | N | C |
| 172 | H | OH | H | H | F-2 | | 0 | F | | H | F | F | C | N | C |
| 173 | H | OH | H | H | F-2 | | 0 | F | | F | H | F | C | N | C |
| 174 | H | OH | H | H | F-2 | | 0 | F | | F | F | H | C | N | C |
| 175 | H | OH | H | H | F-2 | | 0 | H | F | | F | F | C | C | N |
| 176 | H | OH | H | H | F-2 | | 0 | F | H | | F | F | C | C | N |
| 177 | H | OH | H | H | F-2 | | 0 | H | | F | F | H | C | N | C |
| 178 | H | OH | H | H | F-2 | | 0 | F | | H | F | H | C | N | C |
| 179 | H | OH | H | H | F-2 | | 0 | OCF₂CF₂H | H | H | H | H | C | C | C |
| 180 | H | OH | H | H | F-2 | | 0 | H | OCF₂CF₂H | H | H | H | C | C | C |
| 181 | H | OH | H | H | F-2 | | 0 | H | H | OCF₂CF₂H | H | H | C | C | C |
| 182 | H | OH | H | H | F-2 | | 0 | H | OCF₂CF₂H | H | OCF₂CF₂H | H | C | C | C |
| 183 | H | OH | H | H | F-2 | | 0 | OCF₂CF₂H | H | OCF₂CF₂H | H | H | C | C | C |
| 184 | H | OH | H | H | F-2 | | 0 | H | tBu | H | F | H | C | C | C |
| 185 | H | OH | H | H | F-2 | | 0 | H | F | tBu | F | H | C | C | C |
| 186 | H | OH | H | H | F-2 | | 0 | F | H | tBu | H | F | C | C | C |
| 187 | H | OH | H | H | F-2 | C(O) | 1 | F | F | F | H | H | C | C | C |
| 188 | H | OH | H | H | F-2 | C(O) | 1 | F | F | F | F | H | C | C | C |
| 189 | H | OH | H | H | F-2 | C(O) | 1 | F | F | F | F | F | C | C | C |
| 190 | H | OH | H | H | F-2 | C(CF₂)₂ | 1 | F | F | F | H | H | C | C | C |
| 191 | H | OH | H | H | F-2 | C(CF₂)₂ | 1 | F | F | F | F | H | C | C | C |
| 192 | H | OH | H | H | F-2 | C(CF₂)₂ | 1 | F | F | F | F | F | C | C | C |
| 193 | H | OH | H | H | F-2 | O | 1 | F | F | F | F | F | C | C | C |
| 194 | H | OH | H | H | F-2 | O | 1 | F | F | CF3 | F | F | C | C | C |

| No. | R1 | R2 | R3 | R4 | R5 | Y | n | R11 | R12 | R13 | R14 | R15 | R16 | R17 | R18 |
|---|---|---|---|---|---|---|---|---|---|---|---|---|---|---|---|
| 195 | H | OH | H | H | F-3 | | 0 | F-1 | F | F | F | F | F | F | F |
| 196 | H | OH | H | H | F-3 | | 0 | F | F-1 | F | F | F | F | F | F |

In some embodiments, the curing agent is selected from the 197 specific multi-ring resorcinol analog salts of Formula 1 (F-1), including Formula 2 (F-2) or Formula 3 (F-3), shown in Table 4, where the pendant ring(s) of F-1 or F-2 is at R5 of F-1 and the cation is benzyl triphenyl phosphonium (BTPP+).

TABLE 4
Resorcinol Analog Salt Curing Agents with Pendant R5 Ring(s)

| | | Anion | | | | | | | | | | | | | | |
|---|---|---|---|---|---|---|---|---|---|---|---|---|---|---|---|---|
| No. | Cation | R1 | R2 | R3 | R4 | R5 | Y | n | R6 | R7 | R8 | R9 | R10 | C2 | C3 | C4 |
| 1 | BTPP+ | H | O(—) | H | H | F-2 | | 0 | F | H | H | H | H | C | C | C |
| 2 | BTPP+ | H | O(—) | H | H | F-2 | | 0 | H | F | H | H | H | C | C | C |
| 3 | BTPP+ | H | O(—) | H | H | F-2 | | 0 | H | H | F | H | H | C | C | C |
| 4 | BTPP+ | H | O(—) | H | H | F-2 | | 0 | F | F | H | H | H | C | C | C |
| 5 | BTPP+ | H | O(—) | H | H | F-2 | | 0 | F | H | F | H | H | C | C | C |
| 6 | BTPP+ | H | O(—) | H | H | F-2 | | 0 | F | H | H | F | H | C | C | C |
| 7 | BTPP+ | H | O(—) | H | H | F-2 | | 0 | F | H | H | H | F | C | C | C |
| 8 | BTPP+ | H | O(—) | H | H | F-2 | | 0 | F | F | F | H | H | C | C | C |
| 9 | BTPP+ | H | O(—) | H | H | F-2 | | 0 | F | F | H | F | H | C | C | C |
| 10 | BTPP+ | H | O(—) | H | H | F-2 | | 0 | F | F | H | H | F | C | C | C |
| 11 | BTPP+ | H | O(—) | H | H | F-2 | | 0 | F | H | F | F | H | C | C | C |
| 12 | BTPP+ | H | O(—) | H | H | F-2 | | 0 | F | H | F | H | F | C | C | C |
| 13 | BTPP+ | H | O(—) | H | H | F-2 | | 0 | H | F | F | F | H | C | C | C |
| 14 | BTPP+ | H | O(—) | H | H | F-2 | | 0 | F | F | F | F | H | C | C | C |
| 15 | BTPP+ | H | O(—) | H | H | F-2 | | 0 | F | F | F | H | F | C | C | C |
| 16 | BTPP+ | H | O(—) | H | H | F-2 | | 0 | F | F | H | F | F | C | C | C |
| 17 | BTPP+ | H | O(—) | H | H | F-2 | | 0 | F | F | F | F | F | C | C | C |
| 18 | BTPP+ | H | O(—) | H | H | F-2 | | 0 | CF3 | H | H | H | H | C | C | C |
| 19 | BTPP+ | H | O(—) | H | H | F-2 | | 0 | H | CF3 | H | H | H | C | C | C |
| 20 | BTPP+ | H | O(—) | H | H | F-2 | | 0 | H | H | CF3 | H | H | C | C | C |
| 21 | BTPP+ | H | O(—) | H | H | F-2 | | 0 | CF3 | H | CF3 | H | H | C | C | C |
| 22 | BTPP+ | H | O(—) | H | H | F-2 | | 0 | H | CF3 | H | CF3 | H | C | C | C |
| 23 | BTPP+ | H | O(—) | H | H | F-2 | | 0 | H | CF3 | CF3 | H | H | C | C | C |
| 24 | BTPP+ | H | O(—) | H | H | F-2 | | 0 | CF3 | F | H | H | H | C | C | C |
| 25 | BTPP+ | H | O(—) | H | H | F-2 | | 0 | CF3 | H | F | H | H | C | C | C |
| 26 | BTPP+ | H | O(—) | H | H | F-2 | | 0 | CF3 | H | H | F | H | C | C | C |
| 27 | BTPP+ | H | O(—) | H | H | F-2 | | 0 | CF3 | H | H | H | F | C | C | C |
| 28 | BTPP+ | H | O(—) | H | H | F-2 | | 0 | F | CF3 | H | H | H | C | C | C |
| 29 | BTPP+ | H | O(—) | H | H | F-2 | | 0 | H | CF3 | F | H | H | C | C | C |
| 30 | BTPP+ | H | O(—) | H | H | F-2 | | 0 | H | CF3 | H | F | H | C | C | C |
| 31 | BTPP+ | H | O(—) | H | H | F-2 | | 0 | H | CF3 | H | H | F | C | C | C |
| 32 | BTPP+ | H | O(—) | H | H | F-2 | | 0 | F | H | CF3 | H | H | C | C | C |
| 33 | BTPP+ | H | O(—) | H | H | F-2 | | 0 | H | F | CF3 | H | H | C | C | C |
| 34 | BTPP+ | H | O(—) | H | H | F-2 | | 0 | H | H | CF3 | F | H | C | C | C |
| 35 | BTPP+ | H | O(—) | H | H | F-2 | | 0 | H | H | CF3 | H | F | C | C | C |
| 36 | BTPP+ | H | O(—) | H | H | F-2 | | 0 | CF3 | F | F | H | H | C | C | C |
| 37 | BTPP+ | H | O(—) | H | H | F-2 | | 0 | CF3 | F | H | F | H | C | C | C |
| 38 | BTPP+ | H | O(—) | H | H | F-2 | | 0 | CF3 | F | H | H | F | C | C | C |
| 39 | BTPP+ | H | O(—) | H | H | F-2 | | 0 | CF3 | H | F | F | H | C | C | C |
| 40 | BTPP+ | H | O(—) | H | H | F-2 | | 0 | CF3 | H | F | H | F | C | C | C |
| 41 | BTPP+ | H | O(—) | H | H | F-2 | | 0 | CF3 | H | H | F | F | C | C | C |
| 42 | BTPP+ | H | O(—) | H | H | F-2 | | 0 | F | CF3 | F | H | H | C | C | C |
| 43 | BTPP+ | H | O(—) | H | H | F-2 | | 0 | F | CF3 | H | F | H | C | C | C |
| 44 | BTPP+ | H | O(—) | H | H | F-2 | | 0 | F | CF3 | H | H | F | C | C | C |
| 45 | BTPP+ | H | O(—) | H | H | F-2 | | 0 | H | CF3 | F | F | H | C | C | C |
| 46 | BTPP+ | H | O(—) | H | H | F-2 | | 0 | H | CF3 | F | H | F | C | C | C |
| 47 | BTPP+ | H | O(—) | H | H | F-2 | | 0 | H | CF3 | H | F | F | C | C | C |
| 48 | BTPP+ | H | O(—) | H | H | F-2 | | 0 | F | F | CF3 | H | H | C | C | C |
| 49 | BTPP+ | H | O(—) | H | H | F-2 | | 0 | F | H | CF3 | F | H | C | C | C |
| 50 | BTPP+ | H | O(—) | H | H | F-2 | | 0 | F | H | CF3 | H | F | C | C | C |
| 51 | BTPP+ | H | O(—) | H | H | F-2 | | 0 | H | F | CF3 | F | H | C | C | C |
| 52 | BTPP+ | H | O(—) | H | H | F-2 | | 0 | H | F | CF3 | H | F | C | C | C |
| 53 | BTPP+ | H | O(—) | H | H | F-2 | | 0 | CF3 | F | F | F | H | C | C | C |
| 54 | BTPP+ | H | O(—) | H | H | F-2 | | 0 | CF3 | F | F | H | F | C | C | C |
| 55 | BTPP+ | H | O(—) | H | H | F-2 | | 0 | CF3 | F | H | F | F | C | C | C |
| 56 | BTPP+ | H | O(—) | H | H | F-2 | | 0 | F | CF3 | F | F | H | C | C | C |
| 57 | BTPP+ | H | O(—) | H | H | F-2 | | 0 | F | CF3 | F | H | F | C | C | C |
| 58 | BTPP+ | H | O(—) | H | H | F-2 | | 0 | F | CF3 | H | F | F | C | C | C |
| 59 | BTPP+ | H | O(—) | H | H | F-2 | | 0 | H | CF3 | F | F | F | C | C | C |
| 60 | BTPP+ | H | O(—) | H | H | F-2 | | 0 | F | F | CF3 | F | H | C | C | C |
| 61 | BTPP+ | H | O(—) | H | H | F-2 | | 0 | F | F | CF3 | H | F | C | C | C |
| 62 | BTPP+ | H | O(—) | H | H | F-2 | | 0 | F | F | CF3 | F | F | C | C | C |
| 63 | BTPP+ | H | O(—) | H | H | F-2 | | 0 | H | CF3 | F | CF3 | H | C | C | C |
| 64 | BTPP+ | H | O(—) | H | H | F-2 | | 0 | CF3 | F | CF3 | H | H | C | C | C |
| 65 | BTPP+ | H | O(—) | H | H | F-2 | | 0 | OCF3 | H | H | H | H | C | C | C |
| 66 | BTPP+ | H | O(—) | H | H | F-2 | | 0 | H | OCF3 | H | H | H | C | C | C |
| 67 | BTPP+ | H | O(—) | H | H | F-2 | | 0 | H | H | OCF3 | H | H | C | C | C |
| 68 | BTPP+ | H | O(—) | H | H | F-2 | | 0 | OCF3 | H | OCF3 | H | H | C | C | C |
| 69 | BTPP+ | H | O(—) | H | H | F-2 | | 0 | H | OCE3 | OCF3 | H | H | C | C | C |
| 70 | BTPP+ | H | O(—) | H | H | F-2 | | 0 | H | OCF3 | H | OCF3 | H | C | C | C |
| 71 | BTPP+ | H | O(—) | H | H | F-2 | | 0 | OCF3 | F | H | H | H | C | C | C |
| 72 | BTPP+ | H | O(—) | H | H | F-2 | | 0 | OCF3 | H | F | H | H | C | C | C |
| 73 | BTPP+ | H | O(—) | H | H | F-2 | | 0 | OCF3 | H | H | F | H | C | C | C |
| 74 | BTPP+ | H | O(—) | H | H | F-2 | | 0 | OCF3 | H | H | H | F | C | C | C |
| 75 | BTPP+ | H | O(—) | H | H | F-2 | | 0 | F | OCF3 | H | H | H | C | C | C |
| 76 | BTPP+ | H | O(—) | H | H | F-2 | | 0 | H | OCF3 | F | H | H | C | C | C |
| 77 | BTPP+ | H | O(—) | H | H | F-2 | | 0 | H | OCF3 | H | F | H | C | C | C |
| 78 | BTPP+ | H | O(—) | H | H | F-2 | | 0 | H | OCF3 | H | H | F | C | C | C |
| 79 | BTPP+ | H | O(—) | H | H | F-2 | | 0 | F | H | OCF3 | H | H | C | C | C |
| 80 | BTPP+ | H | O(—) | H | H | F-2 | | 0 | H | F | OCF3 | H | H | C | C | C |
| 81 | BTPP+ | H | O(—) | H | H | F-2 | | 0 | H | H | OCF3 | F | H | C | C | C |
| 82 | BTPP+ | H | O(—) | H | H | F-2 | | 0 | H | H | OCF3 | H | F | C | C | C |
| 83 | BTPP+ | H | O(—) | H | H | F-2 | | 0 | OCF3 | F | F | H | H | C | C | C |
| 84 | BTPP+ | H | O(—) | H | H | F-2 | | 0 | OCF3 | F | H | F | H | C | C | C |
| 85 | BTPP+ | H | O(—) | H | H | F-2 | | 0 | OCF3 | F | H | H | F | C | C | C |
| 86 | BTPP+ | H | O(—) | H | H | F-2 | | 0 | OCF3 | H | F | F | H | C | C | C |
| 87 | BTPP+ | H | O(—) | H | H | F-2 | | 0 | OCF3 | H | F | H | F | C | C | C |
| 88 | BTPP+ | H | O(—) | H | H | F-2 | | 0 | OCF3 | H | H | F | F | C | C | C |
| 89 | BTPP+ | H | O(—) | H | H | F-2 | | 0 | F | OCF3 | F | H | H | C | C | C |
| 90 | BTPP+ | H | O(—) | H | H | F-2 | | 0 | F | OCF3 | H | F | H | C | C | C |
| 91 | BTPP+ | H | O(—) | H | H | F-2 | | 0 | F | OCF3 | H | H | F | C | C | C |
| 92 | BTPP+ | H | O(—) | H | H | F-2 | | 0 | H | OCF3 | F | F | H | C | C | C |
| 93 | BTPP+ | H | O(—) | H | H | F-2 | | 0 | H | OCF3 | F | H | F | C | C | C |
| 94 | BTPP+ | H | O(—) | H | H | F-2 | | 0 | H | OCF3 | H | F | F | C | C | C |
| 95 | BTPP+ | H | O(—) | H | H | F-2 | | 0 | F | F | OCF3 | H | H | C | C | C |
| 96 | BTPP+ | H | O(—) | H | H | F-2 | | 0 | F | H | OCF3 | F | H | C | C | C |
| 97 | BTPP+ | H | O(—) | H | H | F-2 | | 0 | F | H | OCF3 | H | F | C | C | C |
| 98 | BTPP+ | H | O(—) | H | H | F-2 | | 0 | H | F | CCF3 | F | H | C | C | C |
| 99 | BTPP+ | H | O(—) | H | H | F-2 | | 0 | H | F | OCF3 | H | F | C | C | C |
| 100 | BTPP+ | H | O(—) | H | H | F-2 | | 0 | OCF3 | F | F | F | H | C | C | C |
| 101 | BTPP+ | H | O(—) | H | H | F-2 | | 0 | OCF3 | F | F | H | F | C | C | C |
| 102 | BTPP+ | H | O(—) | H | H | F-2 | | 0 | OCF3 | F | H | F | F | C | C | C |
| 103 | BTPP+ | H | O(—) | H | H | F-2 | | 0 | F | OCF3 | F | F | H | C | C | C |
| 104 | BTPP+ | H | O(—) | H | H | F-2 | | 0 | F | OCF3 | F | H | F | C | C | C |
| 105 | BTPP+ | H | O(—) | H | H | F-2 | | 0 | F | OCF3 | H | F | F | C | C | C |
| 106 | BTPP+ | H | O(—) | H | H | F-2 | | 0 | H | OCF3 | F | F | F | C | C | C |
| 107 | BTPP+ | H | O(—) | H | H | F-2 | | 0 | F | F | OCF3 | F | H | C | C | C |
| 108 | BTPP+ | H | O(—) | H | H | F-2 | | 0 | F | F | OCF3 | H | F | C | C | C |
| 109 | BTPP+ | H | O(—) | H | H | F-2 | | 0 | F | F | OCF3 | F | F | C | C | C |
| 110 | BTPP+ | H | O(—) | H | H | F-2 | | 0 | H | OCF3 | F | OCF3 | H | C | C | C |
| 111 | BTPP+ | H | O(—) | H | H | F-2 | | 0 | OCF3 | F | OCF3 | H | H | C | C | C |
| 112 | BTPP+ | H | O(—) | H | H | F-2 | | 0 | CF2H | H | H | H | H | C | C | C |
| 113 | BTPP+ | H | O(—) | H | H | F-2 | | 0 | H | CF2H | H | H | H | C | C | C |
| 114 | BTPP+ | H | O(—) | H | H | F-2 | | 0 | H | H | CF2H | H | H | C | C | C |
| 115 | BTPP+ | H | O(—) | H | H | F-2 | | 0 | CF2H | H | CF2H | H | H | C | C | C |
| 116 | BTPP+ | H | O(—) | H | H | F-2 | | 0 | H | CF2H | CF2H | H | H | C | C | C |
| 117 | BTPP+ | H | O(—) | H | H | F-2 | | 0 | H | CF2H | H | CF2H | H | C | C | C |
| 118 | BTPP+ | H | O(—) | H | H | F-2 | | 0 | CF2H | F | H | H | H | C | C | C |
| 119 | BTPP+ | H | O(—) | H | H | F-2 | | 0 | CE2H | H | F | H | H | C | C | C |
| 120 | BTPP+ | H | O(—) | H | H | F-2 | | 0 | CF2H | H | H | F | H | C | C | C |
| 121 | BTPP+ | H | O(—) | H | H | F-2 | | 0 | CE2H | H | H | H | F | C | C | C |
| 122 | BTPP+ | H | O(—) | H | H | F-2 | | 0 | F | CF2H | H | H | H | C | C | C |
| 123 | BTPP+ | H | O(—) | H | H | F-2 | | 0 | H | CF2H | F | H | H | C | C | C |
| 124 | BTPP+ | H | O(—) | H | H | F-2 | | 0 | H | CF2H | H | F | H | C | C | C |
| 125 | BTPP+ | H | O(—) | H | H | F-2 | | 0 | H | CF2H | H | H | F | C | C | C |
| 126 | BTPP+ | H | O(—) | H | H | F-2 | | 0 | F | H | CF2H | H | H | C | C | C |
| 127 | BTPP+ | H | O(—) | H | H | F-2 | | 0 | H | F | CF2H | H | H | C | C | C |
| 128 | BTPP+ | H | O(—) | H | H | F-2 | | 0 | H | H | CF2H | F | H | C | C | C |
| 139 | BTPP+ | H | O(—) | H | H | F-2 | | 0 | H | H | CF2H | H | F | C | C | C |
| 130 | BTPP+ | H | O(—) | H | H | F-2 | | 0 | CF2H | F | F | H | H | C | C | C |
| 131 | BTPP+ | H | O(—) | H | H | F-2 | | 0 | CF2H | F | H | F | H | C | C | C |
| 132 | BTPP+ | H | O(—) | H | H | F-2 | | 0 | CF2H | F | H | H | F | C | C | C |
| 133 | BTPP+ | H | O(—) | H | H | F-2 | | 0 | CF2H | H | F | F | H | C | C | C |
| 134 | BTPP+ | H | O(—) | H | H | F-2 | | 0 | CF2H | H | F | H | F | C | C | C |
| 135 | BTPP+ | H | O(—) | H | H | F-2 | | 0 | CF2H | H | H | F | F | C | C | C |
| 136 | BTPP+ | H | O(—) | H | H | F-2 | | 0 | F | CF2H | F | H | H | C | C | C |
| 137 | BTPP+ | H | O(—) | H | H | F-2 | | 0 | F | CF2H | H | F | H | C | C | C |
| 138 | BTPP+ | H | O(—) | H | H | F-2 | | 0 | F | CF2H | H | H | F | C | C | C |
| 139 | BTPP+ | H | O(—) | H | H | F-2 | | 0 | H | CF2H | F | F | H | C | C | C |
| 140 | BTPP+ | H | O(—) | H | H | F-2 | | 0 | H | CF2H | F | H | F | C | C | C |
| 141 | BTPP+ | H | O(—) | H | H | F-2 | | 0 | H | CF2H | H | F | F | C | C | C |
| 142 | BTPP+ | H | O(—) | H | H | F-2 | | 0 | F | F | CF2H | H | H | C | C | C |
| 143 | BTPP+ | H | O(—) | H | H | F-2 | | 0 | F | H | CF2H | F | H | C | C | C |
| 144 | BTPP+ | H | O(—) | H | H | F-2 | | 0 | F | H | CF2H | H | F | C | C | C |
| 145 | BTPP+ | H | O(—) | H | H | F-2 | | 0 | H | F | CF2H | F | H | C | C | C |
| 146 | BTPP+ | H | O(—) | H | H | F-2 | | 0 | H | F | CF2H | H | F | C | C | C |
| 147 | BTPP+ | H | O(—) | H | H | F-2 | | 0 | CF2H | F | F | F | H | C | C | C |
| 148 | BTPP+ | H | O(—) | H | H | F-2 | | 0 | CF2H | F | F | H | F | C | C | C |
| 149 | BTPP+ | H | O(—) | H | H | F-2 | | 0 | CF2H | F | H | F | F | C | C | C |
| 150 | BTPP+ | H | O(—) | H | H | F-2 | | 0 | F | CF2H | F | F | H | C | C | C |
| 151 | BTPP+ | H | O(—) | H | H | F-2 | | 0 | F | CF2H | F | H | F | C | C | C |
| 152 | BTPP+ | H | O(—) | H | H | F-2 | | 0 | F | CF2H | H | F | F | C | C | C |
| 153 | BTPP+ | H | O(—) | H | H | F-2 | | 0 | H | CF2H | F | F | F | C | C | C |
| 154 | BTPP+ | H | O(—) | H | H | F-2 | | 0 | F | F | CF2H | F | H | C | C | C |
| 155 | BTPP+ | H | O(—) | H | H | F-2 | | 0 | F | F | CF2H | H | F | C | C | C |
| 156 | BTPP+ | H | O(—) | H | H | F-2 | | 0 | F | F | CF2H | F | F | C | C | C |
| 157 | BTPP+ | H | O(—) | H | H | F-2 | | 0 | H | CF2H | F | CF2H | H | C | C | C |
| 158 | BTPP+ | H | O(—) | H | H | F-2 | | 0 | CF2H | F | CF2H | H | H | C | C | C |
| 159 | BTPP+ | H | O(—) | H | H | F-2 | | 0 | C6F5 | H | H | H | H | C | C | C |
| 160 | BTPP+ | H | O(—) | H | H | F-2 | | 0 | H | C6F5 | H | H | H | C | C | C |
| 161 | BTPP+ | H | O(—) | H | H | F-2 | | 0 | H | H | C6F5 | H | H | C | C | C |
| 162 | BTPP+ | H | O(—) | H | H | F-2 | | 0 | F | F | C6F5 | F | F | C | C | C |
| 163 | BTPP+ | H | O(—) | H | H | F-2 | | 0 | F | C6F5 | F | F | F | C | C | C |
| 164 | BTPP+ | H | O(—) | H | H | F-2 | | 0 | C6F5 | F | F | F | F | C | C | C |
| 165 | BTPP+ | H | O(—) | H | H | F-2 | | 0 | | F | F | F | F | N | C | C |
| 166 | BTPP+ | H | O(—) | H | H | F-2 | | 0 | F | | F | F | F | C | N | C |
| 167 | BTPP+ | H | O(—) | H | H | F-2 | | 0 | F | F | | F | F | C | C | N |
| 168 | BTPP+ | H | O(—) | H | H | F-2 | | 0 | | H | F | F | F | N | C | C |
| 169 | BTPP+ | H | O(—) | H | H | F-2 | | 0 | | F | H | F | F | N | C | C |
| 170 | BTPP+ | H | O(—) | H | H | F-2 | | 0 | | F | F | H | F | N | C | C |
| 171 | BTPP+ | H | O(—) | H | H | F-2 | | 0 | | F | F | F | H | N | C | C |
| 172 | BTPP+ | H | O(—) | H | H | F-2 | | 0 | H | | F | F | F | C | N | C |
| 173 | BTPP+ | H | O(—) | H | H | F-2 | | 0 | F | | H | F | F | C | N | C |
| 174 | BTPP+ | H | O(—) | H | H | F-2 | | 0 | F | | F | H | F | C | N | C |
| 175 | BTPP+ | H | O(—) | H | H | F-2 | | 0 | F | | F | F | H | C | N | C |
| 176 | BTPP+ | H | O(—) | H | H | F-2 | | 0 | H | F | | F | F | C | C | N |
| 177 | BTPP+ | H | O(—) | H | H | F-2 | | 0 | F | H | | F | F | C | C | N |
| 178 | BTPP+ | H | O(—) | H | H | F-2 | | 0 | H | | F | F | H | C | N | C |
| 179 | BTPP+ | H | O(—) | H | H | F-2 | | 0 | F | | H | F | H | C | N | C |
| 180 | BTPP+ | H | O(—) | H | H | F-2 | | 0 | OCF₂CF₂H | H | H | H | H | C | C | C |
| 181 | BTPP+ | H | O(—) | H | H | F-2 | | 0 | H | OCF₂CF₂H | H | H | H | C | C | C |
| 182 | BTPP+ | H | O(—) | H | H | F-2 | | 0 | H | H | OCF₂CF₂H | H | H | C | C | C |
| 183 | BTPP+ | H | O(—) | H | H | F-2 | | 0 | H | OCF₂CF₂H | H | OCF₂CF₂H | H | C | C | C |
| 184 | BTPP+ | H | O(—) | H | H | F-2 | | 0 | OCF₂CF₂H | H | OCF₂CF₂H | H | H | C | C | C |
| 185 | BTPP+ | H | O(—) | H | H | F-2 | | 0 | H | tBu | H | F | H | C | C | C |
| 186 | BTPP+ | H | O(—) | H | H | F-2 | | 0 | H | F | tBu | F | H | C | C | C |
| 187 | BTPP+ | H | O(—) | H | H | F-2 | | 0 | F | H | tBu | H | F | C | C | C |
| 188 | BTPP+ | H | O(—) | H | H | F-2 | C(O) | 1 | F | F | F | H | H | C | C | C |
| 189 | BTPP+ | H | O(—) | H | H | F-2 | C(O) | 1 | F | F | F | F | H | C | C | C |
| 190 | BTPP+ | H | O(—) | H | H | F-2 | C(O) | 1 | F | F | F | F | F | C | C | C |
| 191 | BTPP+ | H | O(—) | H | H | F-2 | C(CF₃)₂ | 1 | F | F | F | H | H | C | C | C |
| 192 | BTPP+ | H | O(—) | H | H | F-2 | C(CF₃)₂ | 1 | F | F | F | F | H | C | C | C |
| 193 | BTPP+ | H | O(—) | H | H | F-2 | C(CF₃)₂ | 1 | F | F | F | F | F | C | C | C |
| 194 | BTPP+ | H | O(—) | H | H | F-2 | O | 1 | F | F | F | F | F | C | C | C |
| 195 | BTPP+ | H | O(—) | H | H | F-2 | O | 1 | F | F | CF3 | F | F | C | C | C |

| | | Anion | | | | | | | | | | | | | | |
|---|---|---|---|---|---|---|---|---|---|---|---|---|---|---|---|---|
| No. | Cation | R1 | R2 | R3 | R4 | R5 | Y | n | R11 | R12 | R13 | R14 | R15 | R16 | R17 | R18 |
| 196 | BTPP+ | H | O(—) | H | H | F-3 | | 0 | F-1 | F | F | F | F | F | F | F |
| 197 | BTPP+ | H | O(—) | H | H | F-3 | | 0 | F | F-1 | F | F | F | F | F | F |

In some embodiments, the curing agent is selected from the 168 specific multi-ring resorcinol analogs of Formula 1 (F-1), including Formula 2 (F-2) or Formula 3 (F-3), shown in Table 5, where the pendant ring(s) of F-1 or F-2 is at R4 of F-1.

TABLE 5
Resorcinol Analog Curing Agents with Pendant R4 Ring(s)

| No. | R1 | R2 | R3 | R4 | R5 | Y | n | R6 | R7 | R8 | R9 | R10 | C2 | C3 | C4 |
|---|---|---|---|---|---|---|---|---|---|---|---|---|---|---|---|
| 1 | H | OH | H | F-2 | H | | 0 | F | H | H | H | H | C | C | C |
| 2 | H | OH | H | F-2 | H | | 0 | H | F | H | H | H | C | C | C |
| 3 | H | OH | H | F-2 | H | | 0 | F | F | H | H | H | C | C | C |
| 4 | H | OH | H | F-2 | H | | 0 | F | H | H | F | H | C | C | C |
| 5 | H | OH | H | F-2 | H | | 0 | F | H | H | H | F | C | C | C |
| 6 | H | OH | H | F-2 | H | | 0 | F | F | H | H | F | C | C | C |
| 7 | H | OH | H | F-2 | H | | 0 | F | H | F | H | F | C | C | C |
| 8 | H | OH | H | F-2 | H | | 0 | F | F | F | H | F | C | C | C |
| 9 | H | OH | H | F-2 | H | | 0 | CF3 | H | H | H | H | C | C | C |
| 10 | H | OH | H | F-2 | H | | 0 | H | CF3 | CF3 | H | H | C | C | C |
| 11 | H | OH | H | F-2 | H | | 0 | CF3 | F | H | H | H | C | C | C |
| 12 | H | OH | H | F-2 | H | | 0 | CF3 | H | F | H | H | C | C | C |
| 13 | H | OH | H | F-2 | H | | 0 | CF3 | H | H | F | H | C | C | C |
| 14 | H | OH | H | F-2 | H | | 0 | CF3 | H | H | H | F | C | C | C |
| 15 | H | OH | H | F-2 | H | | 0 | F | CF3 | H | H | H | C | C | C |
| 16 | H | OH | H | F-2 | H | | 0 | H | CF3 | H | H | F | C | C | C |
| 17 | H | OH | H | F-2 | H | | 0 | F | H | CF3 | H | H | C | C | C |
| 18 | H | OH | H | F-2 | H | | 0 | H | H | CF3 | F | H | C | C | C |
| 19 | H | OH | H | F-2 | H | | 0 | H | H | CF3 | H | F | C | C | C |
| 20 | H | OH | H | F-2 | H | | 0 | CF3 | F | F | H | H | C | C | C |
| 21 | H | OH | H | F-2 | H | | 0 | CF3 | F | H | F | H | C | C | C |
| 22 | H | OH | H | F-2 | H | | 0 | CF3 | F | H | H | F | C | C | C |
| 23 | H | OH | H | F-2 | H | | 0 | CF3 | H | F | F | H | C | C | C |
| 24 | H | OH | H | F-2 | H | | 0 | CF3 | H | F | H | F | C | C | C |
| 25 | H | OH | H | F-2 | H | | 0 | CF3 | H | H | F | F | C | C | C |
| 26 | H | OH | H | F-2 | H | | 0 | F | CF3 | F | H | H | C | C | C |
| 27 | H | OH | H | F-2 | H | | 0 | F | CF3 | H | F | H | C | C | C |
| 28 | H | OH | H | F-2 | H | | 0 | F | CF3 | H | H | F | C | C | C |
| 29 | H | OH | H | F-2 | H | | 0 | H | CF3 | F | F | H | C | C | C |
| 30 | H | OH | H | F-2 | H | | 0 | H | CF3 | F | H | F | C | C | C |
| 31 | H | OH | H | F-2 | H | | 0 | H | CF3 | H | F | F | C | C | C |
| 32 | H | OH | H | F-2 | H | | 0 | F | F | CF3 | H | H | C | C | C |
| 33 | H | OH | H | F-2 | H | | 0 | F | H | CF3 | F | H | C | C | C |
| 34 | H | OH | H | F-2 | H | | 0 | F | H | CF3 | H | F | C | C | C |
| 35 | H | OH | H | F-2 | H | | 0 | H | F | CF3 | F | H | C | C | C |
| 36 | H | OH | H | F-2 | H | | 0 | H | F | CF3 | H | F | C | C | C |
| 37 | H | OH | H | F-2 | H | | 0 | CF3 | F | F | F | H | C | C | C |
| 38 | H | OH | H | F-2 | H | | 0 | CF3 | F | F | H | F | C | C | C |
| 39 | H | OH | H | F-2 | H | | 0 | CF3 | F | H | F | F | C | C | C |
| 40 | H | OH | H | F-2 | H | | 0 | F | CF3 | F | F | H | C | C | C |
| 41 | H | OH | H | F-2 | H | | 0 | F | CF3 | F | H | F | C | C | C |
| 42 | H | OH | H | F-2 | H | | 0 | F | CF3 | H | F | F | C | C | C |
| 43 | H | OH | H | F-2 | H | | 0 | H | CF3 | F | F | F | C | C | C |
| 44 | H | OH | H | F-2 | H | | 0 | F | F | CF3 | F | H | C | C | C |
| 45 | H | OH | H | F-2 | H | | 0 | F | F | CF3 | H | F | C | C | C |
| 46 | H | OH | H | F-2 | H | | 0 | F | F | CF3 | F | F | C | C | C |
| 47 | H | OH | H | F-2 | H | | 0 | H | CF3 | F | CF3 | H | C | C | C |
| 48 | H | OH | H | F-2 | H | | 0 | CF3 | F | CF3 | H | H | C | C | C |
| 49 | H | OH | H | F-2 | H | | 0 | OCF3 | H | H | H | H | C | C | C |
| 50 | H | OH | H | F-2 | H | | 0 | OCF3 | H | OCF3 | H | H | C | C | C |
| 51 | H | OH | H | F-2 | H | | 0 | H | OCF3 | OCF3 | H | H | C | C | C |
| 52 | H | OH | H | F-2 | H | | 0 | H | OCF3 | H | OCF3 | H | C | C | C |
| 53 | H | OH | H | F-2 | H | | 0 | OCF3 | F | H | H | H | C | C | C |
| 54 | H | OH | H | F-2 | H | | 0 | OCF3 | H | F | H | H | C | C | C |
| 55 | H | OH | H | F-2 | H | | 0 | OCF3 | H | H | F | H | C | C | C |
| 56 | H | OH | H | F-2 | H | | 0 | OCF3 | H | H | H | F | C | C | C |
| 57 | H | OH | H | F-2 | H | | 0 | F | OCF3 | H | H | H | C | C | C |
| 58 | H | OH | H | F-2 | H | | 0 | H | OCF3 | F | H | H | C | C | C |
| 59 | H | OH | H | F-2 | H | | 0 | H | OCF3 | H | F | H | C | C | C |
| 60 | H | OH | H | F-2 | H | | 0 | H | OCF3 | H | H | F | C | C | C |
| 61 | H | OH | H | F-2 | H | | 0 | F | H | OCF3 | H | H | C | C | C |
| 62 | H | OH | H | F-2 | H | | 0 | H | F | OCF3 | H | H | C | C | C |
| 63 | H | OH | H | F-2 | H | | 0 | H | H | OCF3 | F | H | C | C | C |
| 64 | H | OH | H | F-2 | H | | 0 | H | H | OCF3 | H | F | C | C | C |
| 65 | H | OH | H | F-2 | H | | 0 | OCF3 | F | F | H | H | C | C | C |
| 66 | H | OH | H | F-2 | H | | 0 | OCF3 | F | H | F | H | C | C | C |
| 67 | H | OH | H | F-2 | H | | 0 | OCF3 | F | H | H | F | C | C | C |
| 68 | H | OH | H | F-2 | H | | 0 | OCF3 | H | F | F | H | C | C | C |
| 69 | H | OH | H | F-2 | H | | 0 | OCF3 | H | F | H | F | C | C | C |
| 70 | H | OH | H | F-2 | H | | 0 | OCF3 | H | H | F | F | C | C | C |
| 71 | H | OH | H | F-2 | H | | 0 | F | OCF3 | F | H | H | C | C | C |
| 72 | H | OH | H | F-2 | H | | 0 | F | OCF3 | H | F | H | C | C | C |
| 73 | H | OH | H | F-2 | H | | 0 | F | OCF3 | H | H | F | C | C | C |
| 74 | H | OH | H | F-2 | H | | 0 | H | OCF3 | F | F | H | C | C | C |
| 75 | H | OH | H | F-2 | H | | 0 | H | OCF3 | F | H | F | C | C | C |
| 76 | H | OH | H | F-2 | H | | 0 | H | OCF3 | H | F | F | C | C | C |
| 77 | H | OH | H | F-2 | H | | 0 | F | F | OCF3 | H | H | C | C | C |
| 78 | H | OH | H | F-2 | H | | 0 | F | H | OCF3 | F | H | C | C | C |
| 79 | H | OH | H | F-2 | H | | 0 | F | H | OCF3 | H | F | C | C | C |
| 80 | H | OH | H | F-2 | H | | 0 | H | F | OCF3 | H | F | C | C | C |
| 81 | H | OH | H | F-2 | H | | 0 | OCF3 | F | F | F | H | C | C | C |
| 82 | H | OH | H | F-2 | H | | 0 | OCF3 | F | F | H | F | C | C | C |
| 83 | H | OH | H | F-2 | H | | 0 | OCF3 | F | H | F | F | C | C | C |
| 84 | H | OH | H | F-2 | H | | 0 | F | OCF3 | F | F | H | C | C | C |
| 85 | H | OH | H | F-2 | H | | 0 | F | OCF3 | F | H | F | C | C | C |
| 86 | H | OH | H | F-2 | H | | 0 | F | OCF3 | H | F | F | C | C | C |
| 87 | H | OH | H | F-2 | H | | 0 | H | OCF3 | F | F | F | C | C | C |
| 88 | H | OH | H | F-2 | H | | 0 | F | F | OCF3 | F | H | C | C | C |
| 89 | H | OH | H | F-2 | H | | 0 | F | F | OCF3 | H | F | C | C | C |
| 90 | H | OH | H | F-2 | H | | 0 | F | F | OCF3 | F | F | C | C | C |
| 91 | H | OH | H | F-2 | H | | 0 | H | OCF3 | F | OCF3 | H | C | C | C |
| 92 | H | OH | H | F-2 | H | | 0 | OCF3 | F | OCF3 | H | H | C | C | C |
| 93 | H | OH | H | F-2 | H | | 0 | CF2H | H | H | H | H | C | C | C |
| 94 | H | OH | H | F-2 | H | | 0 | H | CF2H | H | H | H | C | C | C |
| 95 | H | OH | H | F-2 | H | | 0 | H | H | CF2H | H | H | C | C | C |
| 96 | H | OH | H | F-2 | H | | 0 | CF2H | H | CF2H | H | H | C | C | C |
| 97 | H | OH | H | F-2 | H | | 0 | H | CF2H | CF2H | H | H | C | C | C |
| 98 | H | OH | H | F-2 | H | | 0 | H | CF2H | H | CF2H | H | C | C | C |
| 99 | H | OH | H | F-2 | H | | 0 | CF2H | F | H | H | H | C | C | C |
| 100 | H | OH | H | F-2 | H | | 0 | CF2H | H | F | H | H | C | C | C |
| 101 | H | OH | H | F-2 | H | | 0 | CF2H | H | H | F | H | C | C | C |
| 102 | H | OH | H | F-2 | H | | 0 | CF2H | H | H | H | F | C | C | C |
| 103 | H | OH | H | F-2 | H | | 0 | F | CF2H | H | H | H | C | C | C |
| 104 | H | OH | H | F-2 | H | | 0 | H | CF2H | F | H | H | C | C | C |
| 105 | H | OH | H | F-2 | H | | 0 | H | CF2H | H | F | H | C | C | C |
| 106 | H | OH | H | F-2 | H | | 0 | H | CF2H | H | H | F | C | C | C |
| 107 | H | OH | H | F-2 | H | | 0 | F | H | CF2H | H | H | C | C | C |
| 108 | H | OH | H | F-2 | H | | 0 | H | F | CF2H | H | H | C | C | C |
| 109 | H | OH | H | F-2 | H | | 0 | H | H | CF2H | F | H | C | C | C |
| 110 | H | OH | H | F-2 | H | | 0 | H | H | CF2H | H | F | C | C | C |
| 111 | H | OH | H | F-2 | H | | 0 | CF2H | F | F | H | H | C | C | C |
| 112 | H | OH | H | F-2 | H | | 0 | CF2H | F | H | F | H | C | C | C |
| 113 | H | OH | H | F-2 | H | | 0 | CF2H | F | H | H | F | C | C | C |
| 114 | H | OH | H | F-2 | H | | 0 | CF2H | H | F | F | H | C | C | C |
| 115 | H | OH | H | F-2 | H | | 0 | CF2H | H | F | H | F | C | C | C |
| 116 | H | OH | H | F-2 | H | | 0 | CF2H | H | H | F | F | C | C | C |
| 117 | H | OH | H | F-2 | H | | 0 | F | CF2H | F | H | H | C | C | C |
| 118 | H | OH | H | F-2 | H | | 0 | F | CF2H | H | F | H | C | C | C |
| 119 | H | OH | H | F-2 | H | | 0 | F | CF2H | H | H | F | C | C | C |
| 120 | H | OH | H | F-2 | H | | 0 | H | CF2H | F | F | H | C | C | C |
| 121 | H | OH | H | F-2 | H | | 0 | H | CF2H | F | H | F | C | C | C |
| 122 | H | OH | H | F-2 | H | | 0 | H | CF2H | H | F | F | C | C | C |
| 123 | H | OH | H | F-2 | H | | 0 | F | F | CF2H | H | H | C | C | C |
| 124 | H | OH | H | F-2 | H | | 0 | F | H | CF2H | F | H | C | C | C |
| 125 | H | OH | H | F-2 | H | | 0 | F | H | CF2H | H | F | C | C | C |
| 126 | H | OH | H | F-2 | H | | 0 | H | F | CF2H | F | H | C | C | C |
| 127 | H | OH | H | F-2 | H | | 0 | H | F | CF2H | H | F | C | C | C |
| 128 | H | OH | H | F-2 | H | | 0 | CF2H | F | F | F | H | C | C | C |
| 129 | H | OH | H | F-2 | H | | 0 | CF2H | F | F | H | F | C | C | C |
| 130 | H | OH | H | F-2 | H | | 0 | CF2H | F | H | F | F | C | C | C |
| 131 | H | OH | H | F-2 | H | | 0 | F | CF2H | F | F | H | C | C | C |
| 132 | H | OH | H | F-2 | H | | 0 | F | CF2H | F | H | F | C | C | C |
| 133 | H | OH | H | F-2 | H | | 0 | F | CF2H | H | F | F | C | C | C |
| 134 | H | OH | H | F-2 | H | | 0 | H | CF2H | F | F | F | C | C | C |
| 135 | H | OH | H | F-2 | H | | 0 | F | F | CF2H | F | H | C | C | C |
| 136 | H | OH | H | F-2 | H | | 0 | F | F | CF2H | H | F | C | C | C |
| 137 | H | OH | H | F-2 | H | | 0 | F | F | CF2H | F | F | C | C | C |
| 138 | H | OH | H | F-2 | H | | 0 | H | CF2H | F | CF2H | H | C | C | C |
| 139 | H | OH | H | F-2 | H | | 0 | CF2H | F | CF2H | H | H | C | C | C |
| 140 | H | OH | H | F-2 | H | | 0 | C6F5 | H | H | H | H | C | C | C |
| 141 | H | OH | H | F-2 | H | | 0 | H | C6F5 | H | H | H | C | C | C |
| 142 | H | OH | H | F-2 | H | | 0 | H | H | C6F5 | H | H | C | C | C |
| 143 | H | OH | H | F-2 | H | | 0 | F | C6F5 | F | F | F | C | C | C |
| 144 | H | OH | H | F-2 | H | | 0 | C6F5 | F | F | F | F | C | C | C |
| 145 | H | OH | H | F-2 | H | | 0 | F | | F | F | F | C | N | C |
| 146 | H | OH | H | F-2 | H | | 0 | | H | F | F | F | N | C | C |
| 147 | H | OH | H | F-2 | H | | 0 | | F | H | F | F | N | C | C |
| 148 | H | OH | H | F-2 | H | | 0 | | F | F | H | F | N | C | C |
| 149 | H | OH | H | F-2 | H | | 0 | | F | F | F | H | N | C | C |
| 150 | H | OH | H | F-2 | H | | 0 | H | | F | F | F | N | N | C |
| 151 | H | OH | H | F-2 | H | | 0 | F | | H | F | F | C | N | C |
| 152 | H | OH | H | F-2 | H | | 0 | F | | F | H | F | C | N | C |
| 153 | H | OH | H | F-2 | H | | 0 | F | | F | F | H | C | N | C |
| 154 | H | OH | H | F-2 | H | | 0 | H | F | | F | F | C | C | N |
| 155 | H | OH | H | F-2 | H | | 0 | F | H | | F | F | C | C | N |
| 156 | H | OH | H | F-2 | H | | 0 | H | | F | F | H | C | N | C |
| 157 | H | OH | H | F-2 | H | | 0 | F | | H | F | H | C | N | C |
| 158 | H | OH | H | F-2 | H | | 0 | OCF₂CF₂H | H | H | H | H | C | C | C |
| 159 | H | OH | H | F-2 | H | | 0 | H | OCF₂CF₂H | H | H | H | C | C | C |
| 160 | H | OH | H | F-2 | H | | 0 | H | H | OCF₂CF₂H | H | H | C | C | C |
| 161 | H | OH | H | F-2 | H | | 0 | H | OCF₂CF₂H | H | OCF₂CF₂H | H | C | C | C |
| 162 | H | OH | H | F-2 | H | | 0 | OCF₂CF₂H | H | OCF₂CF₂H | H | H | C | C | C |
| 163 | H | OH | H | F-2 | H | | 0 | H | tBu | H | F | H | C | C | C |
| 164 | H | OH | H | F-2 | H | | 0 | H | F | tBu | F | H | C | C | C |
| 165 | H | OH | H | F-2 | H | | 0 | F | H | tBu | H | F | C | C | C |
| 166 | H | OH | H | F-2 | H | O | 1 | F | F | F | F | F | C | C | C |

| No. | R1 | R2 | R3 | R4 | R5 | Y | n | R11 | R12 | R13 | R14 | R15 | R16 | R17 | R18 |
|---|---|---|---|---|---|---|---|---|---|---|---|---|---|---|---|
| 167 | H | OH | H | F-3 | H | | 0 | F-1 | F | F | F | F | F | F | F |
| 168 | H | OH | H | F-3 | H | | 0 | F | F-1 | F | F | F | F | F | F |

In some embodiments, the curing agent is selected from the 191 specific multi-ring resorcinol analog salts of Formula 1 (F-1), including Formula 2 (F-2) or Formula 3 (F-3), shown in Table 6, where the pendant ring(s) of F-1 or F-2 is at R4 of F-1 and the cation is benzyl triphenyl phosphonium (BTPP+).

TABLE 6
Resorcinol Analog Salt Curing Agents with Pendant R4 Ring(s)

| | | Anion | | | | | | | | | | | | | | |
|---|---|---|---|---|---|---|---|---|---|---|---|---|---|---|---|---|
| No. | Cation | R1 | R2 | R3 | R4 | R5 | Y | n | R6 | R7 | R8 | R9 | R10 | C2 | C3 | C4 |
| 1 | BTPP+ | H | O(—) | H | F-2 | H | | 0 | F | H | H | H | H | C | C | C |
| 2 | BTPP+ | H | O(—) | H | F-2 | H | | 0 | H | F | H | H | H | C | C | C |
| 3 | BTPP+ | H | O(—) | H | F-2 | H | | 0 | H | H | F | H | H | C | C | C |
| 4 | BTPP+ | H | O(—) | H | F-2 | H | | 0 | F | F | H | H | H | C | C | C |
| 5 | BTPP+ | H | O(—) | H | F-2 | H | | 0 | F | H | F | H | H | C | C | C |
| 6 | BTPP+ | H | O(—) | H | F-2 | H | | 0 | F | H | H | F | H | C | C | C |
| 7 | BTPP+ | H | O(—) | H | F-2 | H | | 0 | F | H | H | H | F | C | C | C |
| 8 | BTPP+ | H | O(—) | H | F-2 | H | | 0 | F | F | F | H | H | C | C | C |
| 9 | BTPP+ | H | O(—) | H | F-2 | H | | 0 | F | F | H | F | H | C | C | C |
| 10 | BTPP+ | H | O(—) | H | F-2 | H | | 0 | F | F | H | H | F | C | C | C |
| 11 | BTPP+ | H | O(—) | H | F-2 | H | | 0 | F | H | F | F | H | C | C | C |
| 12 | BTPP+ | H | O(—) | H | F-2 | H | | 0 | F | H | F | H | F | C | C | C |
| 13 | BTPP+ | H | O(—) | H | F-2 | H | | 0 | H | F | F | F | H | C | C | C |
| 14 | BTPP+ | H | O(—) | H | F-2 | H | | 0 | F | F | F | F | H | C | C | C |
| 15 | BTPP+ | H | O(—) | H | F-2 | H | | 0 | F | F | F | H | F | C | C | C |
| 16 | BTPP+ | H | O(—) | H | F-2 | H | | 0 | F | F | H | F | F | C | C | C |
| 17 | BTPP+ | H | O(—) | H | F-2 | H | | 0 | F | F | F | F | F | C | C | C |
| 18 | BTPP+ | H | O(—) | H | F-2 | H | | 0 | CF3 | H | H | H | H | C | C | C |
| 19 | BTPP+ | H | O(—) | H | F-2 | H | | 0 | H | CF3 | H | H | H | C | C | C |
| 20 | BTPP+ | H | O(—) | H | F-2 | H | | 0 | H | H | CF3 | H | H | C | C | C |
| 21 | BTPP+ | H | O(—) | H | F-2 | H | | 0 | CF3 | H | CF3 | H | H | C | C | C |
| 22 | BTPP+ | H | O(—) | H | F-2 | H | | 0 | H | CF3 | CF3 | H | H | C | C | C |
| 23 | BTPP+ | H | O(—) | H | F-2 | H | | 0 | H | CF3 | H | CF3 | H | C | C | C |
| 24 | BTPP+ | H | O(—) | H | F-2 | H | | 0 | CF3 | F | H | H | H | C | C | C |
| 25 | BTPP+ | H | O(—) | H | F-2 | H | | 0 | CF3 | H | F | H | H | C | C | C |
| 26 | BTPP+ | H | O(—) | H | F-2 | H | | 0 | CF3 | H | H | F | H | C | C | C |
| 27 | BTPP+ | H | O(—) | H | F-2 | H | | 0 | CF3 | H | H | H | F | C | C | C |
| 28 | BTPP+ | H | O(—) | H | F-2 | H | | 0 | F | CF3 | H | H | H | C | C | C |
| 29 | BTPP+ | H | O(—) | H | F-2 | H | | 0 | H | CF3 | F | H | H | C | C | C |
| 30 | BTPP+ | H | O(—) | H | F-2 | H | | 0 | H | CF3 | H | F | H | C | C | C |
| 31 | BTPP+ | H | O(—) | H | F-2 | H | | 0 | H | CF3 | H | H | F | C | C | C |
| 32 | BTPP+ | H | O(—) | H | F-2 | H | | 0 | F | H | CF3 | H | H | C | C | C |
| 33 | BTPP+ | H | O(—) | H | F-2 | H | | 0 | H | F | CF3 | H | H | C | C | C |
| 34 | BTPP+ | H | O(—) | H | F-2 | H | | 0 | H | H | CF3 | F | H | C | C | C |
| 35 | BTPP+ | H | O(—) | H | F-2 | H | | 0 | H | H | CF3 | H | F | C | C | C |
| 36 | BTPP+ | H | O(—) | H | F-2 | H | | 0 | CF3 | F | F | H | H | C | C | C |
| 37 | BTPP+ | H | O(—) | H | F-2 | H | | 0 | CF3 | F | H | F | H | C | C | C |
| 38 | BTPP+ | H | O(—) | H | F-2 | H | | 0 | CF3 | F | H | H | F | C | C | C |
| 39 | BTPP+ | H | O(—) | H | F-2 | H | | 0 | CF3 | H | F | F | H | C | C | C |
| 40 | BTPP+ | H | O(—) | H | F-2 | H | | 0 | CF3 | H | F | H | F | C | C | C |
| 41 | BTPP+ | H | O(—) | H | F-2 | H | | 0 | CF3 | H | H | F | F | C | C | C |
| 42 | BTPP+ | H | O(—) | H | F-2 | H | | 0 | F | CF3 | F | H | H | C | C | C |
| 43 | BTPP+ | H | O(—) | H | F-2 | H | | 0 | F | CF3 | H | F | H | C | C | C |
| 44 | BTPP+ | H | O(—) | H | F-2 | H | | 0 | F | CF3 | H | H | F | C | C | C |
| 45 | BTPP+ | H | O(—) | H | F-2 | H | | 0 | H | CF3 | F | F | H | C | C | C |
| 46 | BTPP+ | H | O(—) | H | F-2 | H | | 0 | H | CF3 | F | H | F | C | C | C |
| 47 | BTPP+ | H | O(—) | H | F-2 | H | | 0 | H | CF3 | H | F | F | C | C | C |
| 48 | BTPP+ | H | O(—) | H | F-2 | H | | 0 | F | F | CF3 | H | H | C | C | C |
| 49 | BTPP+ | H | O(—) | H | F-2 | H | | 0 | F | H | CF3 | F | H | C | C | C |
| 50 | BTPP+ | H | O(—) | H | F-2 | H | | 0 | F | H | CF3 | H | F | C | C | C |
| 51 | BTPP+ | H | O(—) | H | F-2 | H | | 0 | H | F | CF3 | F | H | C | C | C |
| 52 | BTPP+ | H | O(—) | H | F-2 | H | | 0 | H | F | CF3 | H | F | C | C | C |
| 53 | BTPP+ | H | O(—) | H | F-2 | H | | 0 | CF3 | F | F | F | H | C | C | C |
| 54 | BTPP+ | H | O(—) | H | F-2 | H | | 0 | CF3 | F | F | H | F | C | C | C |
| 55 | BTPP+ | H | O(—) | H | F-2 | H | | 0 | CF3 | F | H | F | F | C | C | C |
| 56 | BTPP+ | H | O(—) | H | F-2 | H | | 0 | F | CF3 | F | F | H | C | C | C |
| 57 | BTPP+ | H | O(—) | H | F-2 | H | | 0 | F | CF3 | F | H | F | C | C | C |
| 58 | BTPP+ | H | O(—) | H | F-2 | H | | 0 | F | CF3 | H | F | F | C | C | C |
| 59 | BTPP+ | H | O(—) | H | F-2 | H | | 0 | H | CF3 | F | F | F | C | C | C |
| 60 | BTPP+ | H | O(—) | H | F-2 | H | | 0 | F | F | CF3 | F | H | C | C | C |
| 61 | BTPP+ | H | O(—) | H | F-2 | H | | 0 | F | F | CF3 | H | F | C | C | C |
| 62 | BTPP+ | H | O(—) | H | F-2 | H | | 0 | F | F | CF3 | F | F | C | C | C |
| 63 | BTPP+ | H | O(—) | H | F-2 | H | | 0 | H | CF3 | F | CF3 | H | C | C | C |
| 64 | BTPP+ | H | O(—) | H | F-2 | H | | 0 | CF3 | F | CF3 | H | H | C | C | C |
| 65 | BTPP+ | H | O(—) | H | F-2 | H | | 0 | OCF3 | H | H | H | H | C | C | C |
| 66 | BTPP+ | H | O(—) | H | F-2 | H | | 0 | H | OCF3 | H | H | H | C | C | C |
| 67 | BTPP+ | H | O(—) | H | F-2 | H | | 0 | H | H | OCF3 | H | H | C | C | C |
| 68 | BTPP+ | H | O(—) | H | F-2 | H | | 0 | OCF3 | H | OCF3 | H | H | C | C | C |
| 69 | BTPP+ | H | O(—) | H | F-2 | H | | 0 | H | OCF3 | OCF3 | H | H | C | C | C |
| 70 | BTPP+ | H | O(—) | H | F-2 | H | | 0 | H | OCF3 | H | OCF3 | H | C | C | C |
| 71 | BTPP+ | H | O(—) | H | F-2 | H | | 0 | OCF3 | F | H | H | H | C | C | C |
| 72 | BTPP+ | H | O(—) | H | F-2 | H | | 0 | OCF3 | H | F | H | H | C | C | C |
| 73 | BTPP+ | H | O(—) | H | F-2 | H | | 0 | OCF3 | H | H | F | H | C | C | C |
| 74 | BTPP+ | H | O(—) | H | F-2 | H | | 0 | OCF3 | H | H | H | F | C | C | C |
| 75 | BTPP+ | H | O(—) | H | F-2 | H | | 0 | F | OCF3 | H | H | H | C | C | C |
| 76 | BTPP+ | H | O(—) | H | F-2 | H | | 0 | H | OCF3 | F | H | H | C | C | C |
| 77 | BTPP+ | H | O(—) | H | F-2 | H | | 0 | H | CCF3 | H | F | H | C | C | C |
| 78 | BTPP+ | H | O(—) | H | F-2 | H | | 0 | H | OCF3 | H | H | F | C | C | C |
| 79 | BTPP+ | H | O(—) | H | F-2 | H | | 0 | F | H | OCF3 | H | H | C | C | C |
| 80 | BTPP+ | H | O(—) | H | F-2 | H | | 0 | H | F | OCF3 | H | H | C | C | C |
| 81 | BTPP+ | H | O(—) | H | F-2 | H | | 0 | H | H | OCF3 | F | H | C | C | C |
| 82 | BTPP+ | H | O(—) | H | F-2 | H | | 0 | H | H | OCF3 | H | F | C | C | C |
| 83 | BTPP+ | H | O(—) | H | F-2 | H | | 0 | OCF3 | F | F | H | H | C | C | C |
| 84 | BTPP+ | H | O(—) | H | F-2 | H | | 0 | OCF3 | F | H | F | H | C | C | C |
| 85 | BTPP+ | H | O(—) | H | F-2 | H | | 0 | OCF3 | F | H | H | F | C | C | C |
| 86 | BTPP+ | H | O(—) | H | F-2 | H | | 0 | OCF3 | H | F | F | H | C | C | C |
| 87 | BTPP+ | H | O(—) | H | F-2 | H | | 0 | OCF3 | H | F | H | F | C | C | C |
| 88 | BTPP+ | H | O(—) | H | F-2 | H | | 0 | OCF3 | H | H | F | F | C | C | C |
| 89 | BTPP+ | H | O(—) | H | F-2 | H | | 0 | F | OCF3 | F | H | H | C | C | C |
| 90 | BTPP+ | H | O(—) | H | F-2 | H | | 0 | F | OCF3 | H | F | H | C | C | C |
| 91 | BTPP+ | H | O(—) | H | F-2 | H | | 0 | F | OCF3 | H | H | F | C | C | C |
| 92 | BTPP+ | H | O(—) | H | F-2 | H | | 0 | H | OCF3 | F | F | H | C | C | C |
| 93 | BTPP+ | H | O(—) | H | F-2 | H | | 0 | H | OCF3 | F | H | F | C | C | C |
| 94 | BTPP+ | H | O(—) | H | F-2 | H | | 0 | H | OCF3 | H | F | F | C | C | C |
| 95 | BTPP+ | H | O(—) | H | F-2 | H | | 0 | F | F | OCF3 | H | H | C | C | C |
| 96 | BTPP+ | H | O(—) | H | F-2 | H | | 0 | F | H | OCF3 | F | H | C | C | C |
| 97 | BTPP+ | H | O(—) | H | F-2 | H | | 0 | F | H | OCF3 | H | F | C | C | C |
| 98 | BTPP+ | H | O(—) | H | F-2 | H | | 0 | H | F | OCF3 | F | H | C | C | C |
| 99 | BTPP+ | H | O(—) | H | F-2 | H | | 0 | H | F | OCF3 | H | F | C | C | C |
| 100 | BTPP+ | H | O(—) | H | F-2 | H | | 0 | OCF3 | F | F | F | H | C | C | C |
| 101 | BTPP+ | H | O(—) | H | F-2 | H | | 0 | OCF3 | F | F | H | F | C | C | C |
| 102 | BTPP+ | H | O(—) | H | F-2 | H | | 0 | OCFS | F | H | F | F | C | C | C |
| 103 | BTPP+ | H | O(—) | H | F-2 | H | | 0 | F | OCF3 | F | F | H | C | C | C |
| 104 | BTPP+ | H | O(—) | H | F-2 | H | | 0 | F | OCF3 | F | H | F | C | C | C |
| 105 | BTPP+ | H | O(—) | H | F-2 | H | | 0 | F | OCF3 | H | F | F | C | C | C |
| 106 | BTPP+ | H | O(—) | H | F-2 | H | | 0 | H | OCF3 | F | F | F | C | C | C |
| 107 | BTPP+ | H | O(—) | H | F-2 | H | | 0 | F | F | OCF3 | F | H | C | C | C |
| 108 | BTPP+ | H | O(—) | H | F-2 | H | | 0 | F | F | OCF3 | H | F | C | C | C |
| 109 | BTPP+ | H | O(—) | H | F-2 | H | | 0 | F | F | OCF3 | F | F | C | C | C |
| 110 | BTPP+ | H | O(—) | H | F-2 | H | | 0 | H | OCF3 | F | OCF3 | H | C | C | C |
| 111 | BTPP+ | H | O(—) | H | F-2 | H | | 0 | OCF3 | F | OCF3 | H | H | C | C | C |
| 112 | BTPP+ | H | O(—) | H | F-2 | H | | 0 | CF2H | H | H | H | H | C | C | C |
| 113 | BTPP+ | H | O(—) | H | F-2 | H | | 0 | H | CF2H | H | H | H | C | C | C |
| 114 | BTPP+ | H | O(—) | H | F-2 | H | | 0 | H | H | CF2H | H | H | C | C | C |
| 115 | BTPP+ | H | O(—) | H | F-2 | H | | 0 | CF2H | H | CF2H | H | H | C | C | C |
| 116 | BTPP+ | H | O(—) | H | F-2 | H | | 0 | H | CF2H | CF2H | H | H | C | C | C |
| 117 | BTPP+ | H | O(—) | H | F-2 | H | | 0 | H | CF2H | H | CF2H | H | C | C | C |
| 118 | BTPP+ | H | O(—) | H | F-2 | H | | 0 | CF2H | F | H | H | H | C | C | C |
| 119 | BTPP+ | H | O(—) | H | F-2 | H | | 0 | CF2H | H | F | H | H | C | C | C |
| 120 | BTPP+ | H | O(—) | H | F-2 | H | | 0 | CF2H | H | H | F | H | C | C | C |
| 121 | BTPP+ | H | O(—) | H | F-2 | H | | 0 | CF2H | H | H | H | F | C | C | C |
| 122 | BTPP+ | H | O(—) | H | F-2 | H | | 0 | F | CF2H | H | H | H | C | C | C |
| 123 | BTPP+ | H | O(—) | H | F-2 | H | | 0 | H | CF2H | F | H | H | C | C | C |
| 124 | BTPP+ | H | O(—) | H | F-2 | H | | 0 | H | CF2H | H | F | H | C | C | C |
| 125 | BTPP+ | H | O(—) | H | F-2 | H | | 0 | H | CF2H | H | H | F | C | C | C |
| 126 | BTPP+ | H | O(—) | H | F-2 | H | | 0 | F | H | CF2H | H | H | C | C | C |
| 127 | BTPP+ | H | O(—) | H | F-2 | H | | 0 | H | F | CF2H | H | H | C | C | C |
| 128 | BTPP+ | H | O(—) | H | F-2 | H | | 0 | H | H | CF2H | F | H | C | C | C |
| 129 | BTPP+ | H | O(—) | H | F-2 | H | | 0 | H | H | CF2H | H | F | C | C | C |
| 130 | BTPP+ | H | O(—) | H | F-2 | H | | 0 | CF2H | F | F | H | H | C | C | C |
| 131 | BTPP+ | H | O(—) | H | F-2 | H | | 0 | CF2H | F | H | F | H | C | C | C |
| 132 | BTPP+ | H | O(—) | H | F-2 | H | | 0 | CF2H | F | H | H | F | C | C | C |
| 133 | BTPP+ | H | O(—) | H | F-2 | H | | 0 | CF2H | H | F | F | H | C | C | C |
| 134 | BTPP+ | H | O(—) | H | F-2 | H | | 0 | CF2H | H | F | H | F | C | C | C |
| 135 | BTPP+ | H | O(—) | H | F-2 | H | | 0 | CF2H | H | H | F | F | C | C | C |
| 136 | BTPP+ | H | O(—) | H | F-2 | H | | 0 | F | CF2H | F | H | H | C | C | C |
| 137 | BTPP+ | H | O(—) | H | F-2 | H | | 0 | F | CF2H | H | F | H | C | C | C |
| 138 | BTPP+ | H | O(—) | H | F-2 | H | | 0 | F | CF2H | H | H | F | C | C | C |
| 139 | BTPP+ | H | O(—) | H | F-2 | H | | 0 | H | CF2H | F | F | H | C | C | C |
| 140 | BTPP+ | H | O(—) | H | F-2 | H | | 0 | H | CF2H | F | H | F | C | C | C |
| 141 | BTPP+ | H | O(—) | H | F-2 | H | | 0 | H | CF2H | H | F | F | C | C | C |
| 142 | BTPP+ | H | O(—) | H | F-2 | H | | 0 | F | F | CF2H | H | H | C | C | C |
| 143 | BTPP+ | H | O(—) | H | F-2 | H | | 0 | F | H | CF2H | F | H | C | C | C |
| 144 | BTPP+ | H | O(—) | H | F-2 | H | | 0 | F | H | CF2H | H | F | C | C | C |
| 145 | BTPP+ | H | O(—) | H | F-2 | H | | 0 | H | F | CF2H | F | H | C | C | C |
| 146 | BTPP+ | H | O(—) | H | F-2 | H | | 0 | H | F | CF2H | H | F | C | C | C |
| 147 | BTPP+ | H | O(—) | H | F-2 | H | | 0 | CF2H | F | F | F | H | C | C | C |
| 148 | BTPP+ | H | O(—) | H | F-2 | H | | 0 | CF2H | F | F | H | F | C | C | C |
| 149 | BTPP+ | H | O(—) | H | F-2 | H | | 0 | CF2H | F | H | F | F | C | C | C |
| 150 | BTPP+ | H | O(—) | H | F-2 | H | | 0 | F | CF2H | F | F | H | C | C | C |
| 151 | BTPP+ | H | O(—) | H | F-2 | H | | 0 | F | CF2H | F | H | F | C | C | C |
| 152 | BTPP+ | H | O(—) | H | F-2 | H | | 0 | F | CF2H | H | F | F | C | C | C |
| 153 | BTPP+ | H | O(—) | H | F-2 | H | | 0 | H | CF2H | F | F | F | C | C | C |
| 154 | BTPP+ | H | O(—) | H | F-2 | H | | 0 | F | F | CF2H | F | H | C | C | C |
| 155 | BTPP+ | H | O(—) | H | F-2 | H | | 0 | F | F | CF2H | H | F | C | C | C |
| 156 | BTPP+ | H | O(—) | H | F-2 | H | | 0 | F | F | CF2H | F | F | C | C | C |
| 157 | BTPP+ | H | O(—) | H | F-2 | H | | 0 | H | CF2H | F | CF2H | H | C | C | C |
| 158 | BTPP+ | H | O(—) | H | F-2 | H | | 0 | CF2H | F | CF2H | H | H | C | C | C |
| 159 | BTPP+ | H | O(—) | H | F-2 | H | | 0 | C6F5 | H | H | H | H | C | C | C |
| 160 | BTPP+ | H | O(—) | H | F-2 | H | | 0 | H | C6F5 | H | H | H | C | C | C |
| 161 | BTPP+ | H | O(—) | H | F-2 | H | | 0 | H | H | C6F5 | H | H | C | C | C |
| 162 | BTPP+ | H | O(—) | H | F-2 | H | | 0 | F | F | C6F5 | F | F | C | C | C |
| 163 | BTPP+ | H | O(—) | H | F-2 | H | | 0 | F | C6F5 | F | F | F | C | C | C |
| 164 | BTPP+ | H | O(—) | H | F-2 | H | | 0 | C6F5 | F | F | F | F | C | C | C |
| 165 | BTPP+ | H | O(—) | H | F-2 | H | | 0 | | F | F | F | F | N | C | C |
| 166 | BTPP+ | H | O(—) | H | F-2 | H | | 0 | F | | F | F | F | C | N | C |
| 167 | BTPP+ | H | O(—) | H | F-2 | H | | 0 | F | F | | F | F | C | C | N |
| 168 | BTPP+ | H | O(—) | H | F-2 | H | | 0 | | H | F | F | F | N | C | C |
| 169 | BTPP+ | H | O(—) | H | F-2 | H | | 0 | | F | H | F | F | N | C | C |
| 170 | BTPP+ | H | O(—) | H | F-2 | H | | 0 | | F | F | H | F | N | C | C |
| 171 | BTPP+ | H | O(—) | H | F-2 | H | | 0 | | F | F | F | H | N | C | C |
| 172 | BTPP+ | H | O(—) | H | F-2 | H | | 0 | H | | F | F | F | C | N | C |
| 173 | BTPP+ | H | O(—) | H | F-2 | H | | 0 | F | | H | F | F | C | N | C |
| 174 | BTPP+ | H | O(—) | H | F-2 | H | | 0 | F | | F | H | F | C | N | C |
| 175 | BTPP+ | H | O(—) | H | F-2 | H | | 0 | F | | F | F | H | C | N | C |
| 176 | BTPP+ | H | O(—) | H | F-2 | H | | 0 | H | F | | F | F | C | C | N |
| 177 | BTPP+ | H | O(—) | H | F-2 | H | | 0 | F | H | | F | F | C | C | N |
| 178 | BTPP+ | H | O(—) | H | F-2 | H | | 0 | H | | F | F | H | C | N | C |
| 179 | BTPP+ | H | O(—) | H | F-2 | H | | 0 | F | | H | F | H | C | N | C |
| 180 | BTPP+ | H | O(—) | H | F-2 | H | | 0 | OCF₂CF₂H | H | H | H | H | C | C | C |
| 181 | BTPP+ | H | O(—) | H | F-2 | H | | 0 | H | OCF₂CF₂H | H | H | H | C | C | C |
| 182 | BTPP+ | H | O(—) | H | F-2 | H | | 0 | H | H | OCF₂CF₂H | H | H | C | C | C |
| 183 | BTPP+ | H | O(—) | H | F-2 | H | | 0 | H | OCF₂CF₂H | H | OCF₂CF₂H | H | C | C | C |
| 184 | BTPP+ | H | O(—) | H | F-2 | H | | 0 | OCF₂CF₂H | H | OCF₂CF₂H | H | H | C | C | C |
| 185 | BTPP+ | H | O(—) | H | F-2 | H | | 0 | H | tBu | H | F | H | C | C | C |
| 186 | BTPP+ | H | O(—) | H | F-2 | H | | 0 | H | F | tBu | F | H | C | C | C |
| 187 | BTPP+ | H | O(—) | H | F-2 | H | | 0 | F | H | tBu | H | F | C | C | C |
| 188 | BTPP+ | H | O(—) | H | F-2 | H | O | 0 | F | F | F | F | F | C | C | C |
| 189 | BTPP+ | H | O(—) | H | F-2 | H | O | 0 | F | F | CF3 | F | F | C | C | C |

| | | Anion | | | | | | | | | | | | | | |
|---|---|---|---|---|---|---|---|---|---|---|---|---|---|---|---|---|
| No. | Cation | R1 | R2 | R3 | R4 | R5 | Y | n | R11 | R12 | R13 | R14 | R15 | R16 | R17 | R18 |
| 190 | BTPP+ | H | O(—) | H | F-3 | H | — | 0 | F-1 | F | F | F | F | F | F | F |
| 191 | BTPP+ | H | O(—) | H | F-3 | H | — | 0 | F | F-1 | F | F | F | F | F | F |

In some embodiments, the curing agent is selected from the 199 specific multi-ring hydroquinone analogs of Formula 1 (F-1), including Formula 2 (F-2) or Formula 3 (F-3), shown in Table 7.

TABLE 7
Hydroquinone Analog Curing Agents with Pendant Ring(s)

| No. | R1 | R2 | R3 | R4 | R5 | Y | n | R6 | R7 | R8 | R9 | R10 | C2 | C3 | C4 |
|---|---|---|---|---|---|---|---|---|---|---|---|---|---|---|---|
| 1 | H | H | OH | F-2 | H | | 0 | F | H | H | H | H | C | C | C |
| 2 | H | H | OH | F-2 | H | | 0 | H | F | H | H | H | C | C | C |
| 3 | H | H | OH | F-2 | H | | 0 | H | H | F | H | H | C | C | C |
| 4 | H | H | OH | F-2 | H | | 0 | F | F | H | H | H | C | C | C |
| 5 | H | H | OH | F-2 | H | | 0 | F | H | F | H | H | C | C | C |
| 6 | H | H | OH | F-2 | H | | 0 | F | H | H | F | H | C | C | C |
| 7 | H | H | OH | F-2 | H | | 0 | F | H | H | H | F | C | C | C |
| 8 | H | H | OH | F-2 | H | | 0 | F | F | F | H | H | C | C | C |
| 9 | H | H | OH | F-2 | H | | 0 | F | F | H | F | H | C | C | C |
| 10 | H | H | OH | F-2 | H | | 0 | F | F | H | H | F | C | C | C |
| 11 | H | H | OH | F-2 | H | | 0 | F | H | F | F | H | C | C | C |
| 12 | H | H | OH | F-2 | H | | 0 | F | H | F | H | F | C | C | C |
| 13 | H | H | OH | F-2 | H | | 0 | H | F | F | F | H | C | C | C |
| 14 | H | H | OH | F-2 | H | | 0 | F | F | F | F | H | C | C | C |
| 15 | H | H | OH | F-2 | H | | 0 | F | F | F | H | F | C | C | C |
| 16 | H | H | OH | F-2 | H | | 0 | F | F | H | F | F | C | C | C |
| 17 | H | H | OH | F-2 | H | | 0 | F | F | F | F | F | C | C | C |
| 18 | H | H | OH | F-2 | H | | 0 | CF3 | H | H | H | H | C | C | C |
| 19 | H | H | OH | F-2 | H | | 0 | H | CF3 | H | H | H | C | C | C |
| 20 | H | H | OH | F-2 | H | | 0 | H | H | CF3 | H | H | C | C | C |
| 21 | H | H | OH | F-2 | H | | 0 | CF3 | H | CF3 | H | H | C | C | C |
| 22 | H | H | OH | F-2 | H | | 0 | H | CF3 | CF3 | H | H | C | C | C |
| 23 | H | H | OH | F-2 | H | | 0 | CF3 | F | H | H | H | C | C | C |
| 24 | H | H | OH | F-2 | H | | 0 | CF3 | H | F | H | H | C | C | C |
| 25 | H | H | OH | F-2 | H | | 0 | CF3 | H | H | F | H | C | C | C |
| 26 | H | H | OH | F-2 | H | | 0 | CF3 | H | H | H | F | C | C | C |
| 27 | H | H | OH | F-2 | H | | 0 | F | CF3 | H | H | H | C | C | C |
| 28 | H | H | OH | F-2 | H | | 0 | H | CF3 | F | H | H | C | C | C |
| 29 | H | H | OH | F-2 | H | | 0 | H | CF3 | H | F | H | C | C | C |
| 30 | H | H | OH | F-2 | H | | 0 | H | CF3 | H | H | F | C | C | C |
| 31 | H | H | OH | F-2 | H | | 0 | F | H | CF3 | H | H | C | C | C |
| 32 | H | H | OH | F-2 | H | | 0 | H | F | CF3 | H | H | C | C | C |
| 33 | H | H | OH | F-2 | H | | 0 | H | H | CF3 | F | H | C | C | C |
| 34 | H | H | OH | F-2 | H | | 0 | H | H | CF3 | H | F | C | C | C |
| 35 | H | H | OH | F-2 | H | | 0 | CF3 | F | F | H | H | C | C | C |
| 36 | H | H | OH | F-2 | H | | 0 | CF3 | F | H | F | H | C | C | C |
| 37 | H | H | OH | F-2 | H | | 0 | CF3 | F | H | H | F | C | C | C |
| 38 | H | H | OH | F-2 | H | | 0 | CF3 | H | F | F | H | C | C | C |
| 39 | H | H | OH | F-2 | H | | 0 | CF3 | H | F | H | F | C | C | C |
| 40 | H | H | OH | F-2 | H | | 0 | CF3 | H | H | F | F | C | C | C |
| 41 | H | H | OH | F-2 | H | | 0 | F | CF3 | F | H | H | C | C | C |
| 42 | H | H | OH | F-2 | H | | 0 | F | CF3 | H | F | H | C | C | C |
| 43 | H | H | OH | F-2 | H | | 0 | F | CF3 | H | H | F | C | C | C |
| 44 | H | H | OH | F-2 | H | | 0 | H | CF3 | F | F | H | C | C | C |
| 45 | H | H | OH | F-2 | H | | 0 | H | CF3 | F | H | F | C | C | C |
| 46 | H | H | OH | F-2 | H | | 0 | H | CF3 | H | F | F | C | C | C |
| 47 | H | H | OH | F-2 | H | | 0 | F | F | CF3 | H | H | C | C | C |
| 48 | H | H | OH | F-2 | H | | 0 | F | H | CF3 | F | H | C | C | C |
| 49 | H | H | OH | F-2 | H | | 0 | F | H | CF3 | H | F | C | C | C |
| 50 | H | H | OH | F-2 | H | | 0 | H | F | CF3 | F | H | C | C | C |
| 51 | H | H | OH | F-2 | H | | 0 | H | F | CF3 | H | F | C | C | C |
| 52 | H | H | OH | F-2 | H | | 0 | CF3 | F | F | F | H | C | C | C |
| 53 | H | H | OH | F-2 | H | | 0 | CF3 | F | F | H | F | C | C | C |
| 54 | H | H | OH | F-2 | H | | 0 | CF3 | F | H | F | F | C | C | C |
| 55 | H | H | OH | F-2 | H | | 0 | F | CF3 | F | F | H | C | C | C |
| 56 | H | H | OH | F-2 | H | | 0 | F | CF3 | F | H | F | C | C | C |
| 57 | H | H | OH | F-2 | H | | 0 | F | CF3 | H | F | F | C | C | C |
| 58 | H | H | OH | F-2 | H | | 0 | H | CF3 | F | F | F | C | C | C |
| 59 | H | H | OH | F-2 | H | | 0 | F | F | CF3 | F | H | C | C | C |
| 60 | H | H | OH | F-2 | H | | 0 | F | F | CF3 | H | F | C | C | C |
| 61 | H | H | OH | F-2 | H | | 0 | F | F | CF3 | F | F | C | C | C |
| 62 | H | H | OH | F-2 | H | | 0 | H | CF3 | F | CF3 | H | C | C | C |
| 63 | H | H | OH | F-2 | H | | 0 | CF3 | F | CF3 | H | H | C | C | C |
| 64 | H | H | OH | F-2 | H | | 0 | OCF3 | H | H | H | H | C | C | C |
| 65 | H | H | OH | F-2 | H | | 0 | H | OCF3 | H | H | H | C | C | C |
| 66 | H | H | OH | F-2 | H | | 0 | H | H | OCF3 | H | H | C | C | C |
| 67 | H | H | OH | F-2 | H | | 0 | OCF3 | H | OCF3 | H | H | C | C | C |
| 68 | H | H | OH | F-2 | H | | 0 | H | OCF3 | OCF3 | H | H | C | C | C |
| 69 | H | H | OH | F-2 | H | | 0 | H | OCF3 | H | OCF3 | H | C | C | C |
| 70 | H | H | OH | F-2 | H | | 0 | OCF3 | F | H | H | H | C | C | C |
| 71 | H | H | OH | F-2 | H | | 0 | OCF3 | H | F | H | H | C | C | C |
| 72 | H | H | OH | F-2 | H | | 0 | OCF3 | H | H | F | H | C | C | C |
| 73 | H | H | OH | F-2 | H | | 0 | OCF3 | H | H | H | F | C | C | C |
| 74 | H | H | OH | F-2 | H | | 0 | F | OCF3 | H | H | H | C | C | C |
| 75 | H | H | OH | F-2 | H | | 0 | H | OCF3 | F | H | H | C | C | C |
| 76 | H | H | OH | F-2 | H | | 0 | H | OCF3 | H | F | H | C | C | C |
| 77 | H | H | OH | F-2 | H | | 0 | H | OCF3 | H | H | F | C | C | C |
| 78 | H | H | OH | F-2 | H | | 0 | F | H | OCF3 | H | H | C | C | C |
| 79 | H | H | OH | F-2 | H | | 0 | H | F | OCF3 | H | H | C | C | C |
| 80 | H | H | OH | F-2 | H | | 0 | H | H | OCF3 | F | H | C | C | C |
| 81 | H | H | OH | F-2 | H | | 0 | H | H | OCF3 | H | F | C | C | C |
| 82 | H | H | OH | F-2 | H | | 0 | OCF3 | F | F | H | H | C | C | C |
| 83 | H | H | OH | F-2 | H | | 0 | OCF3 | F | H | F | H | C | C | C |
| 84 | H | H | OH | F-2 | H | | 0 | OCF3 | F | H | H | F | C | C | C |
| 85 | H | H | OH | F-2 | H | | 0 | OCF3 | H | F | F | H | C | C | C |
| 86 | H | H | OH | F-2 | H | | 0 | OCF3 | H | F | H | F | C | C | C |
| 87 | H | H | OH | F-2 | H | | 0 | OCF3 | H | H | F | F | C | C | C |
| 88 | H | H | OH | F-2 | H | | 0 | F | OCF3 | F | H | H | C | C | C |
| 89 | H | H | OH | F-2 | H | | 0 | F | OCF3 | H | F | H | C | C | C |
| 90 | H | H | OH | F-2 | H | | 0 | F | OCF3 | H | H | F | C | C | C |
| 91 | H | H | OH | F-2 | H | | 0 | H | OCF3 | F | F | H | C | C | C |
| 92 | H | H | OH | F-2 | H | | 0 | H | OCF3 | F | H | F | C | C | C |
| 93 | H | H | OH | F-2 | H | | 0 | H | OCF3 | H | F | F | C | C | C |
| 94 | H | H | OH | F-2 | H | | 0 | F | F | OCF3 | H | H | C | C | C |
| 95 | H | H | OH | F-2 | H | | 0 | F | H | OCF3 | F | H | C | C | C |
| 96 | H | H | OH | F-2 | H | | 0 | F | H | OCF3 | H | F | C | C | C |
| 97 | H | H | OH | F-2 | H | | 0 | H | F | OCF3 | F | H | C | C | C |
| 98 | H | H | OH | F-2 | H | | 0 | H | F | OCF3 | H | F | C | C | C |
| 99 | H | H | OH | F-2 | H | | 0 | OCF3 | F | F | F | H | C | C | C |
| 100 | H | H | OH | F-2 | H | | 0 | OCF3 | F | F | H | F | C | C | C |
| 101 | H | H | OH | F-2 | H | | 0 | OCF3 | F | H | F | F | C | C | C |
| 102 | H | H | OH | F-2 | H | | 0 | F | OCF3 | F | F | H | C | C | C |
| 103 | H | H | OH | F-2 | H | | 0 | F | OCF3 | F | H | F | C | C | C |
| 104 | H | H | OH | F-2 | H | | 0 | F | OCF3 | H | F | F | C | C | C |
| 105 | H | H | OH | F-2 | H | | 0 | H | OCF3 | F | F | F | C | C | C |
| 106 | H | H | OH | F-2 | H | | 0 | F | F | OCF3 | F | H | C | C | C |
| 107 | H | H | OH | F-2 | H | | 0 | F | F | OCF3 | H | F | C | C | C |
| 108 | H | H | OH | F-2 | H | | 0 | F | F | OCF3 | F | F | C | C | C |
| 109 | H | H | OH | F-2 | H | | 0 | H | OCF3 | F | OCF3 | H | C | C | C |
| 110 | H | H | OH | F-2 | H | | 0 | OCF3 | F | OCF3 | H | H | C | C | C |
| 111 | H | H | OH | F-2 | H | | 0 | CF2H | H | H | H | H | C | C | C |
| 112 | H | H | OH | F-2 | H | | 0 | H | CF2H | H | H | H | C | C | C |
| 113 | H | H | OH | F-2 | H | | 0 | H | H | CF2H | H | H | C | C | C |
| 114 | H | H | OH | F-2 | H | | 0 | CF2H | H | CF2H | H | H | C | C | C |
| 115 | H | H | OH | F-2 | H | | 0 | H | CF2H | CF2H | H | H | C | C | C |
| 116 | H | H | OH | F-2 | H | | 0 | H | CF2H | H | CF2H | H | C | C | C |
| 117 | H | H | OH | F-2 | H | | 0 | CF2H | F | H | H | H | C | C | C |
| 118 | H | H | OH | F-2 | H | | 0 | CF2H | H | F | H | H | C | C | C |
| 119 | H | H | OH | F-2 | H | | 0 | CF2H | H | H | F | H | C | C | C |
| 120 | H | H | OH | F-2 | H | | 0 | CF2H | H | H | H | F | C | C | C |
| 121 | H | H | OH | F-2 | H | | 0 | F | CF2H | H | H | H | C | C | C |
| 122 | H | H | OH | F-2 | H | | 0 | H | CF2H | F | H | H | C | C | C |
| 123 | H | H | OH | F-2 | H | | 0 | H | CF2H | H | F | H | C | C | C |
| 124 | H | H | OH | F-2 | H | | 0 | H | CF2H | H | H | F | C | C | C |
| 125 | H | H | OH | F-2 | H | | 0 | F | H | CF2H | H | H | C | C | C |
| 126 | H | H | OH | F-2 | H | | 0 | H | F | CF2H | H | H | C | C | C |
| 127 | H | H | OH | F-2 | H | | 0 | H | H | CF2H | F | H | C | C | C |
| 128 | H | H | OH | F-2 | H | | 0 | H | H | CF2H | H | F | C | C | C |
| 129 | H | H | OH | F-2 | H | | 0 | CF2H | F | F | H | H | C | C | C |
| 130 | H | H | OH | F-2 | H | | 0 | CF2H | F | H | F | H | C | C | C |
| 131 | H | H | OH | F-2 | H | | 0 | CF2H | F | H | H | F | C | C | C |
| 132 | H | H | OH | F-2 | H | | 0 | CF2H | H | F | F | H | C | C | C |
| 133 | H | H | OH | F-2 | H | | 0 | CF2H | H | F | H | F | C | C | C |
| 134 | H | H | OH | F-2 | H | | 0 | CF2H | H | H | F | F | C | C | C |
| 135 | H | H | OH | F-2 | H | | 0 | F | CF2H | F | H | H | C | C | C |
| 136 | H | H | OH | F-2 | H | | 0 | F | CF2H | H | F | H | C | C | C |
| 137 | H | H | OH | F-2 | H | | 0 | F | CF2H | H | H | F | C | C | C |
| 138 | H | H | OH | F-2 | H | | 0 | H | CF2H | F | F | H | C | C | C |
| 139 | H | H | OH | F-2 | H | | 0 | H | CF2H | F | H | F | C | C | C |
| 140 | H | H | OH | F-2 | H | | 0 | H | CF2H | H | F | F | C | C | C |
| 141 | H | H | OH | F-2 | H | | 0 | F | F | CF2H | H | H | C | C | C |
| 142 | H | H | OH | F-2 | H | | 0 | F | H | CF2H | F | H | C | C | C |
| 143 | H | H | OH | F-2 | H | | 0 | F | H | CF2H | H | F | C | C | C |
| 144 | H | H | OH | F-2 | H | | 0 | H | F | CF2H | F | H | C | C | C |
| 145 | H | H | OH | F-2 | H | | 0 | H | F | CF2H | H | F | C | C | C |
| 146 | H | H | OH | F-2 | H | | 0 | CF2H | F | F | F | H | C | C | C |
| 147 | H | H | OH | F-2 | H | | 0 | CF2H | F | F | H | F | C | C | C |
| 148 | H | H | OH | F-2 | H | | 0 | CF2H | F | H | F | F | C | C | C |
| 149 | H | H | OH | F-2 | H | | 0 | F | CF2H | F | F | H | C | C | C |
| 150 | H | H | OH | F-2 | H | | 0 | F | CF2H | F | H | F | C | C | C |
| 151 | H | H | OH | F-2 | H | | 0 | F | CF2H | H | F | F | C | C | C |
| 152 | H | H | OH | F-2 | H | | 0 | H | CF2H | F | F | F | C | C | C |
| 153 | H | H | OH | F-2 | H | | 0 | F | F | CF2H | F | H | C | C | C |
| 154 | H | H | OH | F-2 | H | | 0 | F | F | CF2H | H | F | C | C | C |
| 155 | H | H | OH | F-2 | H | | 0 | F | F | CF2H | F | F | C | C | C |
| 156 | H | H | OH | F-2 | H | | 0 | H | CF2H | F | CF2H | H | C | C | C |
| 157 | H | H | OH | F-2 | H | | 0 | CF2H | F | CF2H | H | H | C | C | C |
| 158 | H | H | OH | F-2 | H | | 0 | C6F5 | H | H | H | H | C | C | C |
| 159 | H | H | OH | F-2 | H | | 0 | H | C6F5 | H | H | H | C | C | C |
| 160 | H | H | OH | F-2 | H | | 0 | H | H | C6F5 | H | H | C | C | C |
| 161 | H | H | OH | F-2 | H | | 0 | F | F | C6F5 | F | F | C | C | C |
| 162 | H | H | OH | F-2 | H | | 0 | F | C6F5 | F | F | F | C | C | C |
| 163 | H | H | OH | F-2 | H | | 0 | C6F5 | F | F | F | F | C | C | C |
| 164 | H | H | OH | F-2 | H | | 0 | — | F | F | F | F | N | C | C |
| 165 | H | H | OH | F-2 | H | | 0 | F | — | F | F | F | C | N | C |
| 166 | H | H | OH | F-2 | H | | 0 | F | F | — | F | F | C | C | N |
| 167 | H | H | OH | F-2 | H | | 0 | — | H | F | F | F | N | C | C |
| 168 | H | H | OH | F-2 | H | | 0 | — | F | H | F | F | N | C | C |
| 169 | H | H | OH | F-2 | H | | 0 | — | F | F | H | F | N | C | C |
| 170 | H | H | OH | F-2 | H | | 0 | — | F | F | F | H | N | C | C |
| 171 | H | H | OH | F-2 | H | | 0 | H | — | F | F | F | C | N | C |
| 172 | H | H | OH | F-2 | H | | 0 | F | — | H | F | F | C | N | C |
| 173 | H | H | OH | F-2 | H | | 0 | F | — | F | H | F | C | N | C |
| 174 | H | H | OH | F-2 | H | | 0 | F | — | F | F | H | C | N | C |
| 175 | H | H | OH | F-2 | H | | 0 | H | F | — | F | F | C | C | N |
| 176 | H | H | OH | F-2 | H | | 0 | F | H | — | F | F | C | C | N |
| 177 | H | H | OH | F-2 | H | | 0 | H | — | F | F | H | C | N | C |
| 178 | H | H | OH | F-2 | H | | 0 | F | — | H | F | H | C | N | C |
| 179 | H | H | OH | F-2 | H | | 0 | OCF₂CF₂H | H | H | H | H | C | C | C |
| 180 | H | H | OH | F-2 | H | | 0 | H | OCF₂CF₂H | H | H | H | C | C | C |
| 181 | H | H | OH | F-2 | H | | 0 | H | H | OCF₂CF₂H | H | H | C | C | C |
| 182 | H | H | OH | F-2 | H | | 0 | H | OCF₂CF₂H | H | OCF₂CF₂H | H | C | C | C |
| 183 | H | H | OH | F-2 | H | | 0 | OCF₂CF₂H | H | OCF₂CF₂H | H | H | C | C | C |
| 184 | H | H | OH | F-2 | H | | 0 | H | tBu | H | F | H | C | C | C |
| 185 | H | H | OH | F-2 | H | | 0 | H | F | tBu | F | H | C | C | C |
| 186 | H | H | OH | F-2 | H | | 0 | F | H | tBu | H | F | C | C | C |
| 187 | H | H | OH | F-2 | H | C(O) | 1 | F | F | F | H | H | C | C | C |
| 188 | H | H | OH | F-2 | H | C(O) | 1 | F | F | F | F | H | C | C | C |
| 189 | H | H | OH | F-2 | H | C(O) | 1 | F | F | F | F | F | C | C | C |
| 190 | H | H | OH | F-2 | H | C(CF₃)₂ | 1 | F | F | F | H | H | C | C | C |
| 191 | H | H | OH | F-2 | H | C(CF₃)₂ | 1 | F | F | F | F | H | C | C | C |
| 192 | H | H | OH | F-2 | H | C(CF₃)₂ | 1 | F | F | F | F | F | C | C | C |
| 193 | H | H | OH | F-2 | H | O | 1 | F | F | F | F | F | C | C | C |
| 194 | H | H | OH | F-2 | H | O | 1 | F | F | CF3 | F | F | C | C | C |
| 195 | F-2 | H | OH | F-2 | H | — | 0 | F | F | F | F | F | C | C | C |
| 196 | F-2 | H | OH | F-2 | H | — | 0 | H | F | F | F | F | C | C | C |
| 197 | F-2 | H | OH | F-2 | H | — | 0 | H | CF3 | H | CF3 | H | C | C | C |

| No. | R1 | R2 | R3 | R4 | R5 | Y | n | R11 | R12 | R13 | R14 | R15 | R16 | R17 | R18 |
|---|---|---|---|---|---|---|---|---|---|---|---|---|---|---|---|
| 198 | H | H | OH | F-3 | H | — | | F-1 | F | F | F | F | F | F | F |
| 199 | H | H | OH | F-3 | H | — | | F | F-1 | F | F | F | F | F | F |

In some embodiments, the curing agent is selected from the 200 specific multi-ring hydroquinone analog salts of Formula 1 (F-1), including Formula 2 (F-2) or Formula 3 (F-3), shown in Table 8, where the cation is benzyl triphenyl phosphonium (BTPP+).

TABLE 8
Hydroquinone Analog Salt Curing Agents with Pendant Ring(s)

| | | Anion | | | | | | | | | | | | | | |
|---|---|---|---|---|---|---|---|---|---|---|---|---|---|---|---|---|
| No. | Cation | R1 | R2 | R3 | R4 | R5 | Y | n | R6 | R7 | R8 | R9 | R10 | C2 | C3 | C4 |
| 1 | BTPP+ | H | H | O(—) | F-2 | H | | 0 | F | H | H | H | H | C | C | C |
| 2 | BTPP+ | H | H | O(—) | F-2 | H | | 0 | H | F | H | H | H | C | C | C |
| 3 | BTPP+ | H | H | O(—) | F-2 | H | | 0 | H | H | F | H | H | C | C | C |
| 4 | BTPP+ | H | H | O(—) | F-2 | H | | 0 | F | F | H | H | H | C | C | C |
| 5 | BTPP+ | H | H | O(—) | F-2 | H | | 0 | F | H | F | H | H | C | C | C |
| 6 | BTPP+ | H | H | O(—) | F-2 | H | | 0 | F | H | H | F | H | C | C | C |
| 7 | BTPP+ | H | H | O(—) | F-2 | H | | 0 | F | H | H | H | F | C | C | C |
| 8 | BTPP+ | H | H | O(—) | F-2 | H | | 0 | F | F | F | H | H | C | C | C |
| 9 | BTPP+ | H | H | O(—) | F-2 | H | | 0 | F | F | H | F | H | C | C | C |
| 10 | BTPP+ | H | H | O(—) | F-2 | H | | 0 | F | F | H | H | F | C | C | C |
| 11 | BTPP+ | H | H | O(—) | F-2 | H | | 0 | F | H | F | F | H | C | C | C |
| 12 | BTPP+ | H | H | O(—) | F-2 | H | | 0 | F | H | F | H | F | C | C | C |
| 13 | BTPP+ | H | H | O(—) | F-2 | H | | 0 | H | F | F | F | H | C | C | C |
| 14 | BTPP+ | H | H | O(—) | F-2 | H | | 0 | F | F | F | F | H | C | C | C |
| 15 | BTPP+ | H | H | O(—) | F-2 | H | | 0 | F | F | F | H | F | C | C | C |
| 16 | BTPP+ | H | H | O(—) | F-2 | H | | 0 | F | F | H | F | F | C | C | C |
| 17 | BTPP+ | H | H | O(—) | F-2 | H | | 0 | F | F | F | F | F | C | C | C |
| 18 | BTPP+ | H | H | O(—) | F-2 | H | | 0 | CF3 | H | H | H | H | C | C | C |
| 19 | BTPP+ | H | H | O(—) | F-2 | H | | 0 | H | CF3 | H | H | H | C | C | C |
| 20 | BTPP+ | H | H | O(—) | F-2 | H | | 0 | H | H | CF3 | H | H | C | C | C |
| 21 | BTPP+ | H | H | O(—) | F-2 | H | | 0 | CF3 | H | CF3 | H | H | C | C | C |
| 22 | BTPP+ | H | H | O(—) | F-2 | H | | 0 | H | CF3 | CF3 | H | H | C | C | C |
| 23 | BTPP+ | H | H | O(—) | F-2 | H | | 0 | H | CF3 | H | CF3 | H | C | C | C |
| 24 | BTPP+ | H | H | O(—) | F-2 | H | | 0 | CF3 | F | H | H | H | C | C | C |
| 25 | BTPP+ | H | H | O(—) | F-2 | H | | 0 | CF3 | H | F | H | H | C | C | C |
| 26 | BTPP+ | H | H | O(—) | F-2 | H | | 0 | CF3 | H | H | F | H | C | C | C |
| 27 | BTPP+ | H | H | O(—) | F-2 | H | | 0 | CF3 | H | H | H | F | C | C | C |
| 28 | BTPP+ | H | H | O(—) | F-2 | H | | 0 | F | CF3 | H | H | H | C | C | C |
| 29 | BTPP+ | H | H | O(—) | F-2 | H | | 0 | H | CF3 | F | H | H | C | C | C |
| 30 | BTPP+ | H | H | O(—) | F-2 | H | | 0 | H | CF3 | H | F | H | C | C | C |
| 31 | BTPP+ | H | H | O(—) | F-2 | H | | 0 | H | CF3 | H | H | F | C | C | C |
| 32 | BTPP+ | H | H | O(—) | F-2 | H | | 0 | F | H | CF3 | H | H | C | C | C |
| 33 | BTPP+ | H | H | O(—) | F-2 | H | | 0 | H | F | CF3 | H | H | C | C | C |
| 34 | BTPP+ | H | H | O(—) | F-2 | H | | 0 | H | H | CF3 | F | H | C | C | C |
| 35 | BTPP+ | H | H | O(—) | F-2 | H | | 0 | H | H | CF3 | H | F | C | C | C |
| 36 | BTPP+ | H | H | O(—) | F-2 | H | | 0 | CF3 | F | F | H | H | C | C | C |
| 37 | BTPP+ | H | H | O(—) | F-2 | H | | 0 | CF3 | F | H | F | H | C | C | C |
| 38 | BTPP+ | H | H | O(—) | F-2 | H | | 0 | CF3 | F | H | H | F | C | C | C |
| 39 | BTPP+ | H | H | O(—) | F-2 | H | | 0 | CF3 | H | F | F | H | C | C | C |
| 40 | BTPP+ | H | H | O(—) | F-2 | H | | 0 | CF3 | H | F | H | F | C | C | C |
| 41 | BTPP+ | H | H | O(—) | F-2 | H | | 0 | CF3 | H | H | F | F | C | C | C |
| 42 | BTPP+ | H | H | O(—) | F-2 | H | | 0 | F | CF3 | F | H | H | C | C | C |
| 43 | BTPP+ | H | H | O(—) | F-2 | H | | 0 | F | CF3 | H | F | H | C | C | C |
| 44 | BTPP+ | H | H | O(—) | F-2 | H | | 0 | F | CF3 | H | H | F | C | C | C |
| 45 | BTPP+ | H | H | O(—) | F-2 | H | | 0 | H | CF3 | F | F | H | C | C | C |
| 46 | BTPP+ | H | H | O(—) | F-2 | H | | 0 | H | CF3 | F | H | F | C | C | C |
| 47 | BTPP+ | H | H | O(—) | F-2 | H | | 0 | H | CF3 | H | F | F | C | C | C |
| 48 | BTPP+ | H | H | O(—) | F-2 | H | | 0 | F | F | CF3 | H | H | C | C | C |
| 49 | BTPP+ | H | H | O(—) | F-2 | H | | 0 | F | H | CF3 | F | H | C | C | C |
| 50 | BTPP+ | H | H | O(—) | F-2 | H | | 0 | F | H | CF3 | H | F | C | C | C |
| 51 | BTPP+ | H | H | O(—) | F-2 | H | | 0 | H | F | CF3 | F | H | C | C | C |
| 52 | BTPP+ | H | H | O(—) | F-2 | H | | 0 | H | F | CF3 | H | F | C | C | C |
| 53 | BTPP+ | H | H | O(—) | F-2 | H | | 0 | CF3 | F | F | F | H | C | C | C |
| 54 | BTPP+ | H | H | O(—) | F-2 | H | | 0 | CF3 | F | F | H | F | C | C | C |
| 55 | BTPP+ | H | H | O(—) | F-2 | H | | 0 | CF3 | F | H | F | F | C | C | C |
| 56 | BTPP+ | H | H | O(—) | F-2 | H | | 0 | F | CF3 | F | F | H | C | C | C |
| 57 | BTPP+ | H | H | O(—) | F-2 | H | | 0 | F | CF3 | F | H | F | C | C | C |
| 58 | BTPP+ | H | H | O(—) | F-2 | H | | 0 | F | CF3 | H | F | F | C | C | C |
| 59 | BTPP+ | H | H | O(—) | F-2 | H | | 0 | H | CF3 | F | F | F | C | C | C |
| 60 | BTPP+ | H | H | O(—) | F-2 | H | | 0 | F | F | CF3 | F | H | C | C | C |
| 61 | BTPP+ | H | H | O(—) | F-2 | H | | 0 | F | F | CF3 | H | F | C | C | C |
| 62 | BTPP+ | H | H | O(—) | F-2 | H | | 0 | F | F | CF3 | F | F | C | C | C |
| 63 | BTPP+ | H | H | O(—) | F-2 | H | | 0 | H | CF3 | F | CF3 | H | C | C | C |
| 64 | BTPP+ | H | H | O(—) | F-2 | H | | 0 | CF3 | F | CF3 | H | H | C | C | C |
| 65 | BTPP+ | H | H | O(—) | F-2 | H | | 0 | OCF3 | H | H | H | H | C | C | C |
| 66 | BTPP+ | H | H | O(—) | F-2 | H | | 0 | H | OCF3 | H | H | H | C | C | C |
| 67 | BTPP+ | H | H | O(—) | F-2 | H | | 0 | H | H | OCF3 | H | H | C | C | C |
| 68 | BTPP+ | H | H | O(—) | F-2 | H | | 0 | OCF3 | H | OCF3 | H | H | C | C | C |
| 69 | BTPP+ | H | H | O(—) | F-2 | H | | 0 | H | OCF3 | OCF3 | H | H | C | C | C |
| 70 | BTPP+ | H | H | O(—) | F-2 | H | | 0 | H | OCF3 | H | OCF3 | H | C | C | C |
| 71 | BTPP+ | H | H | O(—) | F-2 | H | | 0 | OCF3 | F | H | H | H | C | C | C |
| 72 | BTPP+ | H | H | O(—) | F-2 | H | | 0 | OCF3 | H | F | H | H | C | C | C |
| 73 | BTPP+ | H | H | O(—) | F-2 | H | | 0 | OCF3 | H | H | F | H | C | C | C |
| 74 | BTPP+ | H | H | O(—) | F-2 | H | | 0 | OCF3 | H | H | H | F | C | C | C |
| 75 | BTPP+ | H | H | O(—) | F-2 | H | | 0 | F | OCF3 | H | H | H | C | C | C |
| 76 | BTPP+ | H | H | O(—) | F-2 | H | | 0 | H | OCF3 | F | H | H | C | C | C |
| 77 | BTPP+ | H | H | O(—) | F-2 | H | | 0 | H | OCF3 | H | F | H | C | C | C |
| 78 | BTPP+ | H | H | O(—) | F-2 | H | | 0 | H | OCF3 | H | H | F | C | C | C |
| 79 | BTPP+ | H | H | O(—) | F-2 | H | | 0 | F | H | OCF3 | H | H | C | C | C |
| 80 | BTPP+ | H | H | O(—) | F-2 | H | | 0 | H | F | OCF3 | H | H | C | C | C |
| 81 | BTPP+ | H | H | O(—) | F-2 | H | | 0 | H | H | OCF3 | F | H | C | C | C |
| 82 | BTPP+ | H | H | O(—) | F-2 | H | | 0 | H | H | OCF3 | H | F | C | C | C |
| 83 | BTPP+ | H | H | O(—) | F-2 | H | | 0 | OCF3 | F | F | H | H | C | C | C |
| 84 | BTPP+ | H | H | O(—) | F-2 | H | | 0 | OCF3 | F | H | F | H | C | C | C |
| 85 | BTPP+ | H | H | O(—) | F-2 | H | | 0 | OCF3 | F | H | H | F | C | C | C |
| 86 | BTPP+ | H | H | O(—) | F-2 | H | | 0 | OCF3 | H | F | F | H | C | C | C |
| 87 | BTPP+ | H | H | O(—) | F-2 | H | | 0 | OCF3 | H | F | H | F | C | C | C |
| 88 | BTPP+ | H | H | O(—) | F-2 | H | | 0 | OCF3 | H | H | F | F | C | C | C |
| 89 | BTPP+ | H | H | O(—) | F-2 | H | | 0 | F | OCF3 | F | H | H | C | C | C |
| 90 | BTPP+ | H | H | O(—) | F-2 | H | | 0 | F | OCF3 | H | F | H | C | C | C |
| 91 | BTPP+ | H | H | O(—) | F-2 | H | | 0 | F | OCF3 | H | H | F | C | C | C |
| 92 | BTPP+ | H | H | O(—) | F-2 | H | | 0 | H | OCF3 | F | F | H | C | C | C |
| 93 | BTPP+ | H | H | O(—) | F-2 | H | | 0 | H | OCF3 | F | H | F | C | C | C |
| 94 | BTPP+ | H | H | O(—) | F-2 | H | | 0 | H | OCF3 | H | F | F | C | C | C |
| 95 | BTPP+ | H | H | O(—) | F-2 | H | | 0 | F | F | OCF3 | H | H | C | C | C |
| 96 | BTPP+ | H | H | O(—) | F-2 | H | | 0 | F | H | OCF3 | F | H | C | C | C |
| 97 | BTPP+ | H | H | O(—) | F-2 | H | | 0 | F | H | OCF3 | H | F | C | C | C |
| 98 | BTPP+ | H | H | O(—) | F-2 | H | | 0 | H | F | OCF3 | F | H | C | C | C |
| 99 | BTPP+ | H | H | O(—) | F-2 | H | | 0 | H | F | OCF3 | H | F | C | C | C |
| 100 | BTPP+ | H | H | O(—) | F-2 | H | | 0 | OCF3 | F | F | F | H | C | C | C |
| 101 | BTPP+ | H | H | O(—) | F-2 | H | | 0 | OCF3 | F | F | H | F | C | C | C |
| 102 | BTPP+ | H | H | O(—) | F-2 | H | | 0 | OCF3 | F | H | F | F | C | C | C |
| 103 | BTPP+ | H | H | O(—) | F-2 | H | | 0 | F | OCF3 | F | F | H | C | C | C |
| 104 | BTPP+ | H | H | O(—) | F-2 | H | | 0 | F | OCF3 | F | H | F | C | C | C |
| 105 | BTPP+ | H | H | O(—) | F-2 | H | | 0 | F | OCF3 | H | F | F | C | C | C |
| 106 | BTPP+ | H | H | O(—) | F-2 | H | | 0 | H | OCF3 | F | F | F | C | C | C |
| 107 | BTPP+ | H | H | O(—) | F-2 | H | | 0 | F | F | OCF3 | F | H | C | C | C |
| 108 | BTPP+ | H | H | O(—) | F-2 | H | | 0 | F | F | OCE3 | H | F | C | C | C |
| 109 | BTPP+ | H | H | O(—) | F-2 | H | | 0 | F | F | OCF3 | F | F | C | C | C |
| 110 | BTPP+ | H | H | O(—) | F-2 | H | | 0 | H | OCF3 | F | OCF3 | H | C | C | C |
| 111 | BTPP+ | H | H | O(—) | F-2 | H | | 0 | OCF3 | F | OCF3 | H | H | C | C | C |
| 112 | BTPP+ | H | H | O(—) | F-2 | H | | 0 | CF2H | H | H | H | H | C | C | C |
| 113 | BTPP+ | H | H | O(—) | F-2 | H | | 0 | H | CF2H | H | H | H | C | C | C |
| 114 | BTPP+ | H | H | O(—) | F-2 | H | | 0 | H | H | CF2H | H | H | C | C | C |
| 115 | BTPP+ | H | H | O(—) | F-2 | H | | 0 | CF2H | H | CF2H | H | H | C | C | C |
| 116 | BTPP+ | H | H | O(—) | F-2 | H | | 0 | H | CF2H | CF2H | H | H | C | C | C |
| 117 | BTPP+ | H | H | O(—) | F-2 | H | | 0 | H | CF2H | H | CF2H | H | C | C | C |
| 118 | BTPP+ | H | H | O(—) | F-2 | H | | 0 | CF2H | F | H | H | H | C | C | C |
| 119 | BTPP+ | H | H | O(—) | F-2 | H | | 0 | CF2H | H | F | H | H | C | C | C |
| 120 | BTPP+ | H | H | O(—) | F-2 | H | | 0 | CF2H | H | H | F | H | C | C | C |
| 121 | BTPP+ | H | H | O(—) | F-2 | H | | 0 | CF2H | H | H | H | F | C | C | C |
| 122 | BTPP+ | H | H | O(—) | F-2 | H | | 0 | F | CF2H | H | H | H | C | C | C |
| 123 | BTPP+ | H | H | O(—) | F-2 | H | | 0 | H | CF2H | F | H | H | C | C | C |
| 124 | BTPP+ | H | H | O(—) | F-2 | H | | 0 | H | CF2H | H | F | H | C | C | C |
| 125 | BTPP+ | H | H | O(—) | F-2 | H | | 0 | H | CF2H | H | H | F | C | C | C |
| 126 | BTPP+ | H | H | O(—) | F-2 | H | | 0 | F | H | CF2H | H | H | C | C | C |
| 127 | BTPP+ | H | H | O(—) | F-2 | H | | 0 | H | F | CF2H | H | H | C | C | C |
| 128 | BTPP+ | H | H | O(—) | F-2 | H | | 0 | H | H | CF2H | F | H | C | C | C |
| 129 | BTPP+ | H | H | O(—) | F-2 | H | | 0 | H | H | CF2H | H | F | C | C | C |
| 130 | BTPP+ | H | H | O(—) | F-2 | H | | 0 | CF2H | F | F | H | H | C | C | C |
| 131 | BTPP+ | H | H | O(—) | F-2 | H | | 0 | CF2H | F | H | F | H | C | C | C |
| 132 | BTPP+ | H | H | O(—) | F-2 | H | | 0 | CF2H | F | H | H | F | C | C | C |
| 133 | BTPP+ | H | H | O(—) | F-2 | H | | 0 | CF2H | H | F | F | H | C | C | C |
| 134 | BTPP+ | H | H | O(—) | F-2 | H | | 0 | CF2H | H | F | H | F | C | C | C |
| 135 | BTPP+ | H | H | O(—) | F-2 | H | | 0 | CF2H | H | H | F | F | C | C | C |
| 136 | BTPP+ | H | H | O(—) | F-2 | H | | 0 | F | CF2H | F | H | H | C | C | C |
| 137 | BTPP+ | H | H | O(—) | F-2 | H | | 0 | F | CF2H | H | F | H | C | C | C |
| 138 | BTPP+ | H | H | O(—) | F-2 | H | | 0 | F | CF2H | H | H | F | C | C | C |
| 139 | BTPP+ | H | H | O(—) | F-2 | H | | 0 | H | CF2H | F | F | H | C | C | C |
| 140 | BTPP+ | H | H | O(—) | F-2 | H | | 0 | H | CF2H | F | H | F | C | C | C |
| 141 | BTPP+ | H | H | O(—) | F-2 | H | | 0 | H | CF2H | H | F | F | C | C | C |
| 142 | BTPP+ | H | H | O(—) | F-2 | H | | 0 | F | F | CF2H | H | H | C | C | C |
| 143 | BTPP+ | H | H | O(—) | F-2 | H | | 0 | F | H | CF2H | F | H | C | C | C |
| 144 | BTPP+ | H | H | O(—) | F-2 | H | | 0 | F | H | CF2H | H | F | C | C | C |
| 145 | BTPP+ | H | H | O(—) | F-2 | H | | 0 | H | F | CF2H | F | H | C | C | C |
| 146 | BTPP+ | H | H | O(—) | F-2 | H | | 0 | H | F | CF2H | H | F | C | C | C |
| 147 | BTPP+ | H | H | O(—) | F-2 | H | | 0 | CF2H | F | F | F | H | C | C | C |
| 148 | BTPP+ | H | H | O(—) | F-2 | H | | 0 | CF2H | F | F | H | F | C | C | C |
| 149 | BTPP+ | H | H | O(—) | F-2 | H | | 0 | CF2H | F | H | F | F | C | C | C |
| 150 | BTPP+ | H | H | O(—) | F-2 | H | | 0 | F | CF2H | F | F | H | C | C | C |
| 151 | BTPP+ | H | H | O(—) | F-2 | H | | 0 | F | CF2H | F | H | F | C | C | C |
| 152 | BTPP+ | H | H | O(—) | F-2 | H | | 0 | F | CF2H | H | F | F | C | C | C |
| 153 | BTPP+ | H | H | O(—) | F-2 | H | | 0 | H | CF2H | F | F | F | C | C | C |
| 154 | BTPP+ | H | H | O(—) | F-2 | H | | 0 | F | F | CF2H | F | H | C | C | C |
| 155 | BTPP+ | H | H | O(—) | F-2 | H | | 0 | F | F | CF2H | H | F | C | C | C |
| 156 | BTPP+ | H | H | O(—) | F-2 | H | | 0 | F | F | CF2H | F | F | C | C | C |
| 157 | BTPP+ | H | H | O(—) | F-2 | H | | 0 | H | CF2H | F | CF2H | H | C | C | C |
| 158 | BTPP+ | H | H | O(—) | F-2 | H | | 0 | CF2H | F | CF2H | H | H | C | C | C |
| 159 | BTPP+ | H | H | O(—) | F-2 | H | | 0 | C6F5 | H | H | H | H | C | C | C |
| 160 | BTPP+ | H | H | O(—) | F-2 | H | | 0 | H | C6F5 | H | H | H | C | C | C |
| 161 | BTPP+ | H | H | O(—) | F-2 | H | | 0 | H | H | C6F5 | H | H | C | C | C |
| 162 | BTPP+ | H | H | O(—) | F-2 | H | | 0 | F | F | C6F5 | F | F | C | C | C |
| 163 | BTPP+ | H | H | O(—) | F-2 | H | | 0 | F | C6F5 | F | F | F | C | C | C |
| 164 | BTPP+ | H | H | O(—) | F-2 | H | | 0 | C6F5 | F | F | F | F | C | C | C |
| 165 | BTPP+ | H | H | O(—) | F-2 | H | | 0 | | F | F | F | F | N | C | C |
| 166 | BTPP+ | H | H | O(—) | F-2 | H | | 0 | F | — | F | F | F | C | N | C |
| 167 | BTPP+ | H | H | O(—) | F-2 | H | | 0 | F | F | — | F | F | C | C | N |
| 168 | BTPP+ | H | H | O(—) | F-2 | H | | 0 | | H | F | F | F | N | C | C |
| 169 | BTPP+ | H | H | O(—) | F-2 | H | | 0 | | F | H | F | F | N | C | C |
| 170 | BTPP+ | H | H | O(—) | F-2 | H | | 0 | | F | F | H | F | N | C | C |
| 171 | BTPP+ | H | H | O(—) | F-2 | H | | 0 | | F | F | F | H | N | C | C |
| 172 | BTPP+ | H | H | O(—) | F-2 | H | | 0 | H | | F | F | F | C | N | C |
| 173 | BTPP+ | H | H | O(—) | F-2 | H | | 0 | F | | H | F | F | C | N | C |
| 174 | BTPP+ | H | H | O(—) | F-2 | H | | 0 | F | | F | H | F | C | N | C |
| 175 | BTPP+ | H | H | O(—) | F-2 | H | | 0 | F | | F | F | H | C | N | C |
| 176 | BTPP+ | H | H | O(—) | F-2 | H | | 0 | H | F | | F | F | C | C | N |
| 177 | BTPP+ | H | H | O(—) | F-2 | H | | 0 | F | H | | F | F | C | C | N |
| 178 | BTPP+ | H | H | O(—) | F-2 | H | | 0 | H | | F | F | H | C | N | C |
| 179 | BTPP+ | H | H | O(—) | F-2 | H | | 0 | F | | H | F | H | C | N | C |
| 180 | BTPP+ | H | H | O(—) | F-2 | H | | 0 | OCF₂CF₂H | H | H | H | H | C | C | C |
| 181 | BTPP+ | H | H | O(—) | F-2 | H | | 0 | H | OCF₂CF₂H | H | H | H | C | C | C |
| 182 | BTPP+ | H | H | O(—) | F-2 | H | | 0 | H | H | OCF₂CF₂H | H | H | C | C | C |
| 183 | BTPP+ | H | H | O(—) | F-2 | H | | 0 | H | OCF₂CF₂H | H | OCF₂CF₂H | H | C | C | C |
| 184 | BTPP+ | H | H | O(—) | F-2 | H | | 0 | OCF₂CF₂H | H | OCF₂CF₂H | H | H | C | C | C |
| 185 | BTPP+ | H | H | O(—) | F-2 | H | | 0 | H | tBu | H | F | H | C | C | C |
| 186 | BTPP+ | H | H | O(—) | F-2 | H | | 0 | H | F | tBu | F | H | C | C | C |
| 187 | BTPP+ | H | H | O(—) | F-2 | H | | 0 | F | H | tBu | H | F | C | C | C |
| 188 | BTPP+ | H | H | O(—) | F-2 | H | C(O) | 1 | F | F | F | H | H | C | C | C |
| 189 | BTPP+ | H | H | O(—) | F-2 | H | C(O) | 1 | F | F | F | F | H | C | C | C |
| 190 | BTPP+ | H | H | O(—) | F-2 | H | C(O) | 1 | F | F | F | F | F | C | C | C |
| 191 | BTPP+ | H | H | O(—) | F-2 | H | C(CF₃)₂ | 1 | F | F | F | H | H | C | C | C |
| 192 | BTPP+ | H | H | O(—) | F-2 | H | C(CF₃)₂ | 1 | F | F | F | F | H | C | C | C |
| 193 | BTPP+ | H | H | O(—) | F-2 | H | C(CF₃)₂ | 1 | F | F | F | F | F | C | C | C |
| 194 | BTPP+ | H | H | O(—) | F-2 | H | O | 1 | F | F | F | F | F | C | C | C |
| 195 | BTPP+ | H | H | O(—) | F-2 | H | O | 1 | F | F | CF3 | F | F | C | C | C |
| 196 | BTPP+ | F-2 | H | O(—) | F-2 | H | | 0 | F | F | F | F | F | C | C | C |
| 197 | BTPP+ | F-2 | H | O(—) | F-2 | H | | 0 | H | F | F | F | F | C | C | C |
| 198 | BTPP+ | F-2 | H | O(—) | F-2 | H | | 0 | H | CF3 | H | CF3 | H | C | C | C |

| | | Anion | | | | | | | | | | | | | | |
|---|---|---|---|---|---|---|---|---|---|---|---|---|---|---|---|---|
| No. | Cation | R1 | R2 | R3 | R4 | R5 | Y | n | R11 | R12 | R13 | R14 | R15 | R16 | R17 | R18 |
| 198 | BTPP+ | H | H | O(—) | F-3 | H | | 0 | F-1 | F | F | F | F | F | F | F |
| 200 | BTPP+ | H | H | O(—) | F-3 | H | | 0 | F | F-1 | F | F | F | F | F | F |

In some embodiments, the curing agent is a fluorinated resorcinol analog. Exemplary fluorinated resorcinol analogs may include, but are not limited to:

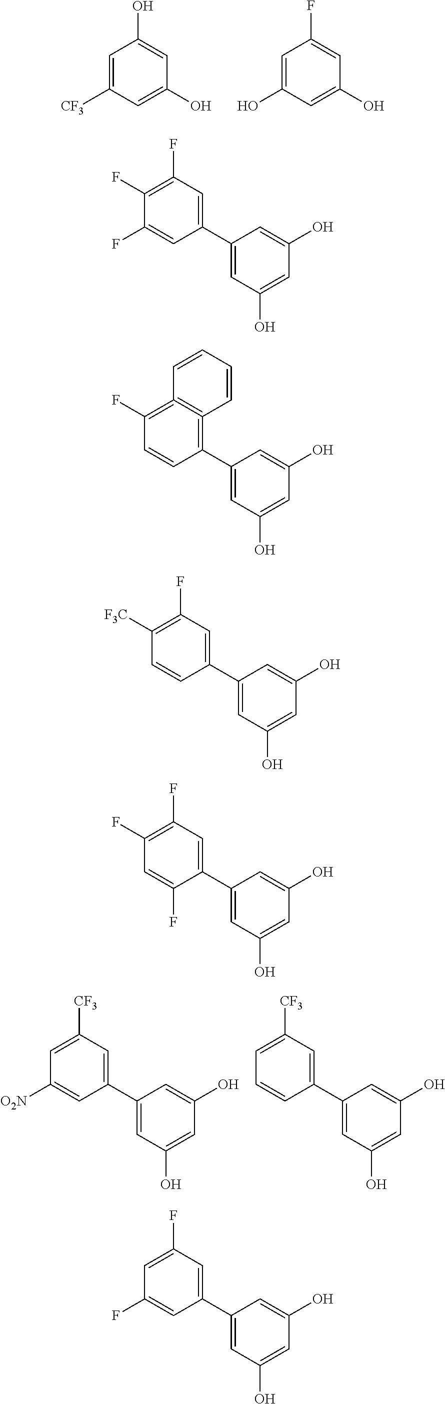

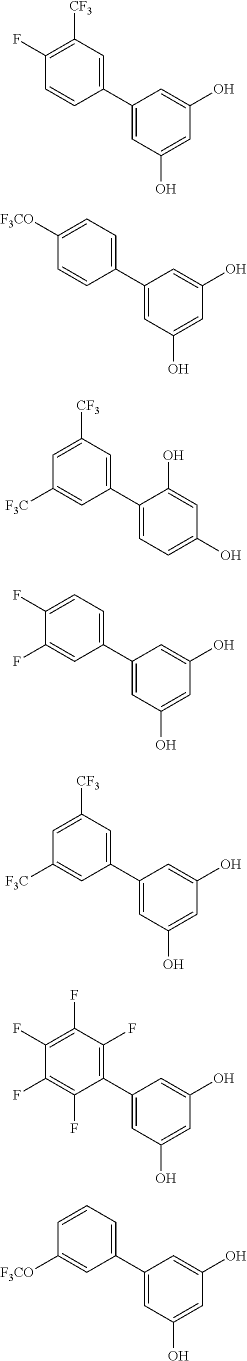

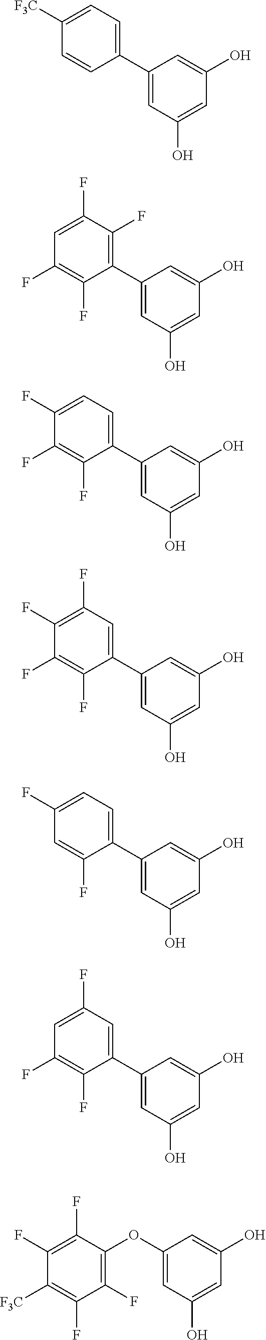

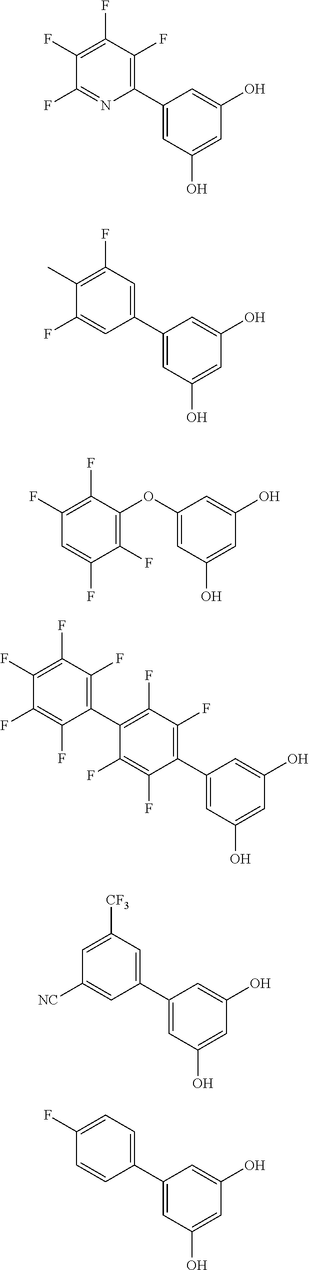

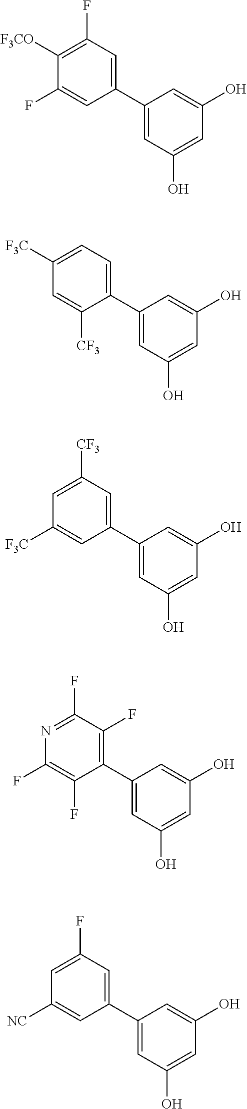 [SMILES: Oc1cc(O)cc(-c2cc(F)c(OC(F)(F)F)c(F)c2)c1 ; Oc1cc(O)cc(-c2ccc(C(F)(F)F)cc2C(F)(F)F)c1 ; Oc1cc(O)cc(-c2cc(C(F)(F)F)cc(C(F)(F)F)c2)c1 ; Oc1cc(O)cc(-c2c(F)c(F)nc(F)c2F)c1 ; N#Cc1cc(F)cc(-c2cc(O)cc(O)c2)c1]

In some embodiments, the curing agent is a fluorinated hydroquinone analog. Exemplary fluorinated hydroquinone analogs include, but are not limited to:

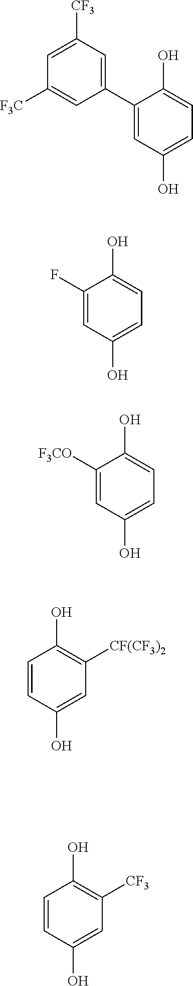

In some embodiments, the curing agent is selected from the following structures:

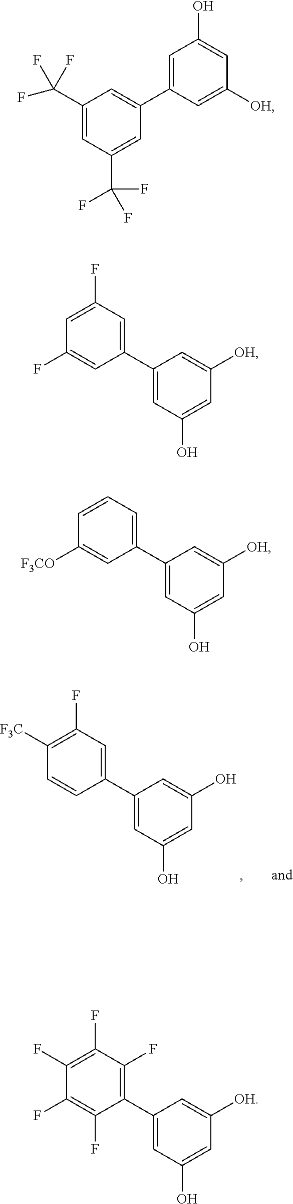 [SMILES: Oc1cc(O)cc(-c2cc(C(F)(F)F)cc(C(F)(F)F)c2)c1 ; Oc1cc(O)cc(-c2cc(F)cc(F)c2)c1 ; Oc1cc(O)cc(-c2cccc(OC(F)(F)F)c2)c1 ; Oc1cc(O)cc(-c2ccc(C(F)(F)F)c(F)c2)c1 ; Oc1cc(O)cc(-c2c(F)c(F)c(F)c(F)c2F)c1]

In some embodiments, the curing agent is part of a curable fluoroelastomer composition that further includes a polyhydroxy-curable fluoroelastomer and an acid acceptor.

In some embodiments, the curable fluoroelastomer composition includes about 0.1 to about 10 parts by weight of the curing agent per 100 parts by weight of fluoroelastomer, alternatively about 0.2 to about 5 parts by weight, alternatively about 0.5 to about 5 parts by weight, alternatively about 1 to about 2.4 parts by weight, or any value, range, or sub-range therebetween.

The fluoroelastomer may be any polyhydroxy-curable fluoroelastomer. As used herein, “polyhydroxy-curable” refers to fluoroelastomers that are known to crosslink with polyhydroxy curing agents such as BPAF. Such fluoroelastomers include, but are not limited to, those having a plurality of carbon-carbon double bonds along the main elastomer polymer chain and also fluoroelastomers that contain sites that may be readily dehydrofluorinated. The latter fluoroelastomers include, but are not limited to, those that contain adjacent copolymerized units of vinylidene fluoride (VF2) and hexafluoropropylene (HFP) as well as fluoroelastomers that contain adjacent copolymerized units of VF2 (or tetrafluoroethylene) and a fluorinated comonomer having an acidic hydrogen atom, such as, for example, 2-hydropentafluoropropylene; 1-hydropentafluoropropylene; trifluoroethylene; 2,3,3,3-tetrafluoropropene; or 3,3,3-trifluoropropene. Preferred fluoroelastomers include the copolymers of i) vinylidene fluoride with hexafluoropropylene and, optionally, tetrafluoroethylene (TFE); ii) vinylidene fluoride with a perfluoro(alkyl vinyl ether) such as perfluoro(methyl vinyl ether), 2-hydropentafluoropropylene and optionally, tetrafluoroethylene; iii) tetrafluoroethylene with propylene and 3,3,3-trifluoropropene; iv) tetrafluoroethylene, perfluoro(methyl vinyl ether) and hexafluoro-2-(pentafluorophenoxy)-1-(trifluorovinyloxy)propane, and v) ethylene with tetrafluoroethylene, perfluoro(methyl vinyl ether) and 3,3,3-trifluoropropylene. In some embodiments, the polyhydroxy-curable fluoroelastomer is a dipolymer of hexafluoropropylene and vinylidene fluoride. Polyhydroxy-curable fluoroelastomers may also include iodine-, bromine-, or chlorine-containing elastomers. For example, small amounts (0.01-1 wt %) of chlorine, bromine, or iodine can be introduced with telogens such as, for example, CH₂I₂ or I(CF₂)₄I, or monomers such as, for example, CH₂═CHCF₂CF₂X (X═Br, I) or chlorotrifluoroethylene. In some embodiments, the polyhydroxy-curable fluoroelastomer contains a bis-olefin, such as, for example, CH₂═CH(CF₂)ₙCH═CH₂ (where n=2-8) or CF₂═CFO(CF₂)ₙOCF═CF₂ (where n=2-8).

Appropriate acid acceptors may include, but are not limited to, powdered magnesium oxide, calcium hydroxide, zinc oxide, bismuth oxide, lead oxide, calcium oxide, hydrotalcite, barium carbonate, calcium carbonate, alkyl stearates, or a combination thereof. In some embodiments, the curable fluoroelastomer composition includes about 3 to about 15 parts by weight of the acid acceptor per 100 parts by weight of fluoroelastomer, alternatively about 5 to about 15 parts by weight, alternatively about 6 to about 12 parts by weight, alternatively about 8 to about 10 parts by weight, or any value, range, or sub-range therebetween. In some embodiments, a composition includes two or more acid acceptors.

In some embodiments the curable composition includes an organic base. Appropriate organic bases may include, but are not limited to, 1,8-diazobicyclo[5,4,0]undec-7-ene (DBU) or salts thereof, 1,5-diazabicyclo(4.3.0)-non-5-ene (DBN) or salts thereof, or a combination thereof.

In some embodiments, the curable composition includes one or more additives. Appropriate additives may include, but are not limited to, processing aids and/or colorants.

In some embodiments, a fluoroelastomer masterbatch includes the curing agent and a polyhydroxy-curable fluoropolymer.

In some embodiments, a curing agent and curing accelerator mixture includes the curing agent and a curing accelerator.

Appropriate curing accelerators may include, but are not limited to, tertiary sulfonium salts such as [(C₆H₅)₂S⁺(C₆H₁₃)][Cl]⁻, and [(C₆H₁₃)₂S(C₆H₅)]⁺[CH₃CO₂]⁻ and quaternary ammonium, phosphonium, arsonium, and stibonium salts of the formula R₅R₆R₇R₈Y⁺X⁻, where Y is phosphorous, nitrogen, arsenic, or antimony; R₅, R₆, R₇, and R₈ are individually C₁-C₂₀ alkyl, aryl, aralkyl, alkenyl, and the chlorine, fluorine, bromine, cyano, —OR, and —COOR substituted analogs thereof, with R being C₁-C₂₀ alkyl, aryl, aralkyl, alkenyl, and where X is halide, hydroxide, sulfate, sulfite, carbonate, pentachlorothiophenolate, tetrafluoroborate, hexafluorosilicate, hexafluorophosphate, dimethyl phosphate, and C₁-C₂₀ alkyl, aryl, aralkyl, and alkenyl carboxylates and dicarboxylates. Particularly preferred are benzyltriphenylphosphonium chloride, benzyltriphenylphosphonium bromide, tetrabutylammonium hydrogen sulfate, tetrabutylammonium hydroxide, tetrapropylammonium hydroxide, tetrabutylammonium bromide, tributylallylphosphonium chloride, tributyl-2-methoxypropylphosphonium chloride, 1,8-diazabicyclo[5.4.0]undec-7-ene, and benzyldiphenyl(dimethylamino)phosphonium chloride. Other appropriate curing accelerators include methyltrioctylammonium chloride, methyltributylammonium chloride, tetrapropylammonium chloride, benzyltrioctylphosphonium bromide, benzyltrioctylphosphonium chloride, methyltrioctylphosphonium acetate, tetraoctylphosphonium bromide, methyltriphenylarsonium tetrafluoroborate, tetraphenylstibonium bromide, 4-chlorobenzyltriphenyl phosphonium chloride, 8-benzyl-1,8-diazabicyclo(5.4.0)-7-undecenonium chloride, diphenylmethyltriphenylphosphonium chloride, allyltriphenyl-phosphonium chloride, tetrabutylphosphonium bromide, m-trifluoromethyl-benzyltrioctylphosphonium chloride, and other quaternary compounds disclosed in U.S. Pat. Nos. 5,591,804; 4,912,171; 4,882,390; 4,259,463; 4,250,278 and 3,876,654.

In some embodiments, the curing accelerator includes a quaternary phosphonium salt, a quaternary ammonium salt, or a tertiary sulfonium salt.

In some embodiments, the curing accelerator includes benzyl triphenyl phosphonium chloride or tetrabutylammonium hydrogen sulfate.

In some embodiments, the curing agent and the curing accelerator in the curing agent and curing accelerator mixture are at a weight ratio in the range of about 1:1 to about 12:1, alternatively about 1.5:1 to about 10:1, alternatively about 2:1 to about 8:1, or any value, range, or sub-range therebetween.

In some embodiments, the curing agent and curing accelerator are pre-reacted to form a salt of the curing agent and curing accelerator. In some embodiments, the curing agent is in a twofold to sixfold molar excess, alternatively in a twofold to fivefold molar excess, alternatively in a threefold to sixfold molar excess, alternatively in about a 3.5-fold molar excess, alternatively in about a 5.3-fold molar excess, or any value, range, or sub-range therebetween, with respect to the amount of curing accelerator.

In some embodiments, the pre-reacted curing agent-curing accelerator salt provides similar curing properties to but significantly better compression sets for the cured polymer than the same curing agent and curing accelerator not in a pre-reacted salt. In some embodiments, the compression sets are reduced by at least 10% with the pre-reacted salt. In some embodiments, the compression sets are reduced by at least 30% with the pre-reacted salt. In some embodiments, the compression sets are reduced by a greater percentage than when BPAF is used as the curing agent.

In some embodiments, a phenoxide derived from a curing agent of Formula 1 is in the form of a quaternary phosphonium salt or quaternary ammonium salt for use as a fluoroelastomer curing agent and curing accelerator.

In some embodiments, a process cures a polyhydroxy-curable fluoropolymer with the curing agent.

In some embodiments, the process includes forming a mixture of the polyhydroxy-curable fluoropolymer, the curing agent, at least one acid acceptor, and a curing accelerator.

In some embodiments, the mixture includes about 0.05 to about 1.5 parts by weight of the curing accelerator per 100 parts by weight of fluoroelastomer, alternatively about 0.1 to about 1 part by weight, alternatively about 0.2 to about 0.8 parts by weight, alternatively about 0.25 to about 0.6 parts by weight, or any value, range, or sub-range therebetween.

In some embodiments, the mixture further comprises a filler. The filler may one or more inorganic fillers, one or more polymeric fillers, or combinations thereof. In some embodiments, the filler is a medium thermal carbon black. Other appropriate inorganic fillers may include, but are not limited to, silica, talc, titanium dioxide (TiO₂), barium sulfate (BaSO₄), calcium carbonate (CaCO₃), or a combination thereof. Appropriate polymeric fillers may include, but are not limited to, polytetrafluoroethylene (PTFE). In some embodiments, the mixture includes about 10 to about 40 parts by weight of the filler per 100 parts by weight of fluoroelastomer, alternatively about 20 to about 40 parts by weight, alternatively about 25 to about 35 parts by weight, alternatively about 30 parts by weight, or any value, range, or sub-range therebetween.

In some embodiments, the curing temperature is in the range of about 150° C. to about 200° C., alternatively about 160° C. to about 190° C., alternatively about 170° C. to about 180ºC, or any value, range, or sub-range therebetween.

In some embodiments, the curing time is in the range of about 5 to about 60 minutes, alternatively about 5 to about 20 minutes, alternatively about 10 to about 30 minutes, alternatively about 20 to about 30 minutes, or any value, range, or sub-range therebetween.

In some embodiments, the curing agent provides cure properties that are similar to the cure properties of BPAF. Such properties may include, but are not limited to, M_{L}, M_{H}, ts1, ts2, t₅₀, and t₉₀. In some embodiments, the values are within 50%, alternatively within 40%, alternatively within 30%, alternatively within 20%, alternatively within 10%, alternatively within 5%, or any value, range, or sub-range therebetween, of the values for BPAF as a curing agent.

In some embodiments, the curing agent provides a cured fluoroelastomer having similar properties to a cured fluoroelastomer formed with BPAF as the curing agent. Such properties may include, but are not limited to, compression set resistance, tensile strength, elongation at break, and elastic modulus at 100%. In some embodiments, the values are within 50%, alternatively within 40%, alternatively within 30%, alternatively within 20%, alternatively within 10%, alternatively within 5%, or any value, range, or sub-range therebetween, of the values for BPAF as a curing agent.

In some embodiments, a method of curing a polyhydroxy-curable fluoroelastomer includes forming a curable fluoroelastomer composition including a polyhydroxy-curable fluoroelastomer, a curing agent of Formula 1, and an acid acceptor and heating the curable fluoroelastomer composition to cure the polyhydroxy-curable fluoroelastomer.

In some embodiments, the curable fluoroelastomer composition is free of or substantially free of 2,2-bis(4 hydroxyphenyl)hexafluoropropane.

In some embodiments, an article is cured by the method of curing.

In some embodiments, the article is free of or substantially free of 2,2-bis(4 hydroxyphenyl)hexafluoropropane.

In another embodiment, a compound is of Formula 1A:

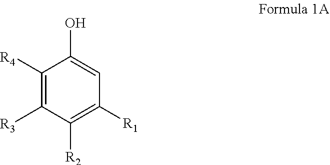

where one of R₁ and R₂ is H and the other is OH. One of R₃ and R₄ is H and the other is selected from the group consisting of Formula 2A and Formula 3A:

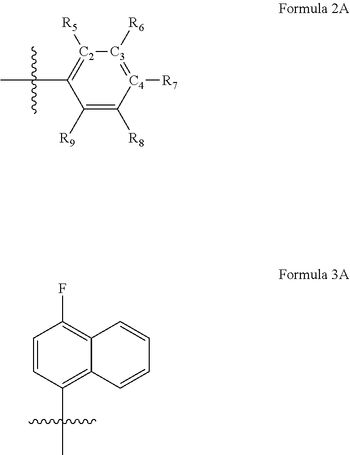

where R₅, R₆, R₇, R₈, and Ry are independently selected from the group consisting of H, F, CF₃, partially or fully fluorinated phenyl, OCF₃, CH₃, nitro, and nitrile; with the proviso that at least one of R₅, R₆, R₇, R₈, and R₉ is selected from the group consisting of F, CF₃, partially or fully fluorinated phenyl, and OCF₃; with the proviso that when R₅, R₆, R₇, R₈, and R₉ are independently selected from the group consisting of H and F, at least two are H and at least two are F; with the proviso that when R₁ is OH, R₃ is Formula 2A, and R₅, R₆, R₇, R₈, and R₉ are independently selected from the group consisting of H and CF₃, at least one of R₆ and R₈ is H; and with the proviso that N may be substituted at exactly one of C₂, C₃, or C₄ in which case respective R₅, R₆, or R₇ is absent.

Applications of the cured fluoropolymers described herein may include, but are not limited to, sealing materials, shaft seals, O-rings, containers, hoses, or wearable applications, such as, for example, wristwatch bands.

In some embodiments, the fluoroelastomers described are blended with one or more other fluoroelastomers or polymers to form a polymer blend. Appropriate blend polymers include, but are not limited to, nylon or other polyamides.

Although the curing agents have been described herein for curing curable fluoroelastomers, the curing agents may have other applications as well. In some embodiments, the curing agents are reacted with polyisocyanates to form polyurethanes.

In other embodiments, the curing agents are included in polyesters. In some such embodiments, the curing agents are condensed with aliphatic dicarboxylic acids or aromatic dicarboxylic acids, such as, for example, terephthalic acid, isophthalic acid, or mixtures thereof, or their esters to form aliphatic-aromatic polyesters or aromatic-aromatic polyesters, respectively. The resulting polymers may be amorphous, high-T_{g} materials or liquid crystalline aromatic polyesters. The introduction of the fluorinated aromatic side groups may result in good polymer processibility, good thermal stability, and/or good oxidative stability.

In other embodiments, the curing agents are included in polyimides, polyamides, polycarbonates, and/or epoxy resins.

### Test Methods

### Moving Die Rheometer (MDR) Measurements

Cure properties were measured on fluoroelastomer curing compositions of about 8 grams following ASTM D5289 on an MDR-2000 Rheometer (Alpha Technologies, Bellingham, WA). The curing temperature was 177° C., and the curing time was 24 minutes. The moving die frequency was 1.66 Hz, and the oscillation amplitude was 0.5°.

Reported cure properties include M_{L} in dN·m, M_{H} in dN·m, ts1 in minutes, ts2 in minutes, t₅₀ in minutes, and too in minutes.

### Fluoroelastomer Property Measurements

Compression set resistances were determined on the fluoroelastomers with a compression device that compressed fluoroelastomer samples to 25% deflection following ASTM D395, Test Method B. Prior to the compression set testing, the fluoroelastomer was post-cured for 16 hours at 232° C. The compression set resistance is reported as a percentage change in thickness after a predetermined time at a predetermined temperature. Three values are reported herein: at 70 hours at 200° C. (CS1), at 168 hours at 200° C. (CS2), and at 70 hours at 250° C. (CS3).

Tensile properties were determined on the unaged fluoroelastomers at 23° C. by the ISO 37:2005 C or 1 2008 testing protocol. Prior to the tensile testing, the fluoroelastomer was post-cured for 16 hours at 232° C. Measured tensile properties included the tensile strength in MPa, the elongation at break in %, and the elastic modulus at 100% in MPa.

Fluid ageing was performed on certain fluoroelastomer O-rings under the condition of placing them in sulfuric acid at 70° C. for 168 hours. Prior to the fluid ageing, the fluoroelastomer was post-cured for 16 hours at 232° C. Following the fluid ageing, swelling of the fluoroelastomer as a weight percent was measured and a compression set (CS1) was performed on the sample.

### EXAMPLES

### Synthesis Examples

Twenty-nine fluorinated resorcinol or hydroquinone analogs were prepared for evaluation as curing agents. The chemical structures of these Inventive Examples (IEs) are shown in Table 9.

The starting material for Inventive Example 1 was acquired from MilliporeSigma (Burlington, MA) and further purified by column chromatography on silica gel.

The fluorinated resorcinol analogs of Inventive Examples 2-12 and 15-19, 21-27, and 29 and the fluorinated hydroquinone analog of Inventive Example 20 were prepared by a palladium catalyst-based synthesis approach often used for coupling of aryl-boronic acids with arylbromides as building blocks. When the aromatic hydroxy groups of the building blocks were protected with methyl groups, an additional hydrolysis step of the methoxy groups was used to make the fluorinated resorcinol and hydroquinone analogs.

For example, in the synthesis of Inventive Example 2, 3,5-dimethoxy-phenylboronic acid (12.5 g), 1-bromo-2,3,5-trifluorobenzene (11 g), potassium carbonate (13.8 g), Pd(PPh₃)₄ (0.36 g), water (44 g), and toluene (132 g), were stirred and refluxed under nitrogen for 4 hours. The resulting toluene solution was separated and dried over MgSO₄. Toluene was removed by distillation and the resulting (MeO)₂C₆H₃—C₆F₃H₂ was distilled under vacuum (120-142° C./0.6-0.8 Torr). Then, the (MeO)₂C₆H₃—C₆F₃H₂ (6.3 g) was diluted in dichloromethane (22 g), and 1M BBr₃ solution in dichloromethane (52 mL) was added at a temperature of −7 to 0° C. and allowed to stir and warm to room temperature overnight. The mixture was then cooled to 0° C.; water was carefully added dropwise; and the product was extracted with ethyl acetate. The extract was dried and filtered and the solvent was evaporated to obtain a final yellow powder (4.4 g, m.p.=187° C., 3,5-(HO)₂C₆H₃—C₆F₃H₂) of Inventive Example 2.

TABLE 9
Chemical Formulas of Inventive Examples

| | Formula |
|---|---|
| 1 | 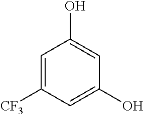 [SMILES: Oc1cc(O)cc(C(F)(F)F)c1] |
| 2 | 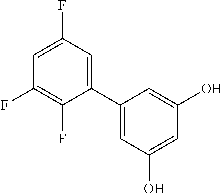 [SMILES: Oc1cc(O)cc(-c2cc(F)cc(F)c2F)c1] |
| 3 | 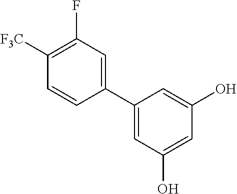 [SMILES: Oc1cc(O)cc(-c2ccc(C(F)(F)F)c(F)c2)c1] |
| 4 | 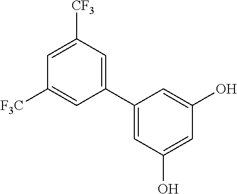 [SMILES: Oc1cc(O)cc(-c2cc(C(F)(F)F)cc(C(F)(F)F)c2)c1] |
| 5 | 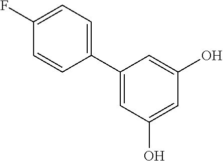 [SMILES: Oc1cc(O)cc(-c2ccc(F)cc2)c1] |
| 6 | 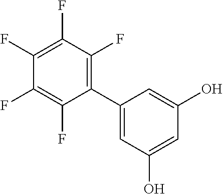 [SMILES: Oc1cc(O)cc(-c2c(F)c(F)c(F)c(F)c2F)c1] |
| 7 | 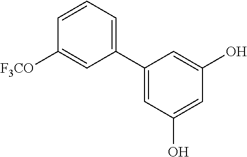 [SMILES: Oc1cc(O)cc(-c2cccc(OC(F)(F)F)c2)c1] |
| 8 | 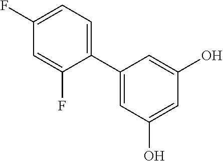 [SMILES: Oc1cc(O)cc(-c2ccc(F)cc2F)c1] |
| 9 | 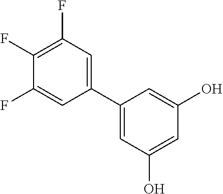 [SMILES: Oc1cc(O)cc(-c2cc(F)c(F)c(F)c2)c1] |
| 10 | 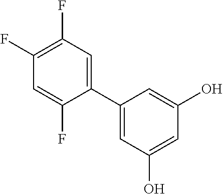 [SMILES: Oc1cc(O)cc(-c2cc(F)c(F)cc2F)c1] |
| 11 | 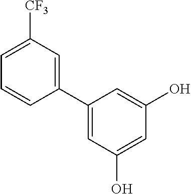 [SMILES: Oc1cc(O)cc(-c2cccc(C(F)(F)F)c2)c1] |
| 12 | 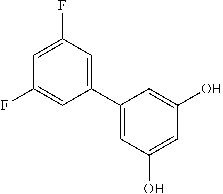 [SMILES: Oc1cc(O)cc(-c2cc(F)cc(F)c2)c1] |
| 13 | 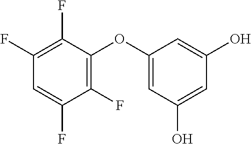 [SMILES: Oc1cc(O)cc(Oc2c(F)c(F)cc(F)c2F)c1] |
| 14 | 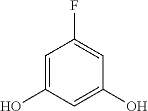 |
| 15 | 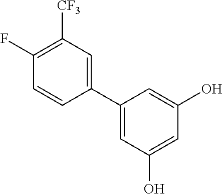 [SMILES: Oc1cc(O)cc(-c2ccc(F)c(C(F)(F)F)c2)c1] |
| 16 | 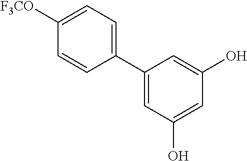 [SMILES: Oc1cc(O)cc(-c2ccc(OC(F)(F)F)cc2)c1] |
| 17 | 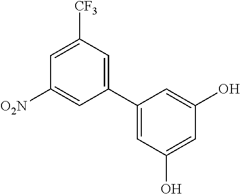 [SMILES: O=[N+]([O-])c1cc(-c2cc(O)cc(O)c2)cc(C(F)(F)F)c1] |
| 18 | 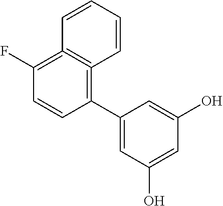 |
| 19 | 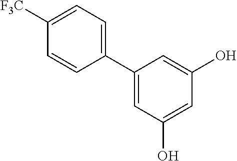 [SMILES: Oc1cc(O)cc(-c2ccc(C(F)(F)F)cc2)c1] |
| 20 | 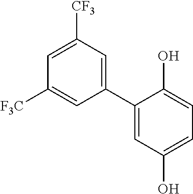 [SMILES: Oc1ccc(O)c(-c2cc(C(F)(F)F)cc(C(F)(F)F)c2)c1] |
| 21 | 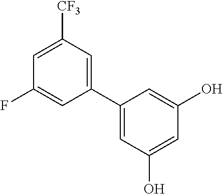 [SMILES: Oc1cc(O)cc(-c2cc(F)cc(C(F)(F)F)c2)c1] |
| 22 | 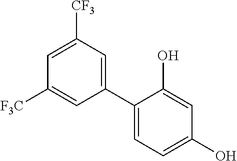 [SMILES: Oc1ccc(-c2cc(C(F)(F)F)cc(C(F)(F)F)c2)c(O)c1] |
| 23 | 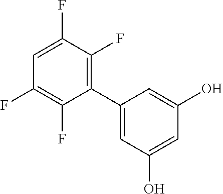 [SMILES: Oc1cc(O)cc(-c2c(F)c(F)cc(F)c2F)c1] |
| 24 | 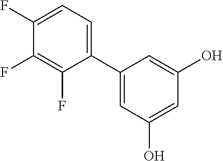 [SMILES: Oc1cc(O)cc(-c2ccc(F)c(F)c2F)c1] |
| 25 | 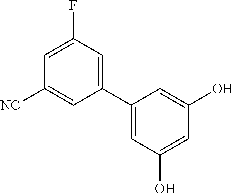 [SMILES: N#Cc1cc(F)cc(-c2cc(O)cc(O)c2)c1] |
| 26 | 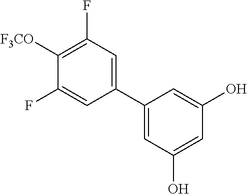 [SMILES: Oc1cc(O)cc(-c2cc(F)c(OC(F)(F)F)c(F)c2)c1] |
| 27 | 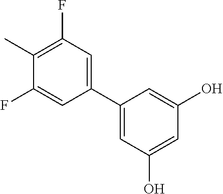 [SMILES: Cc1c(F)cc(-c2cc(O)cc(O)c2)cc1F] |
| 28 | 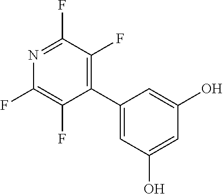 [SMILES: Oc1cc(O)cc(-c2c(F)c(F)nc(F)c2F)c1] |
| 29 | 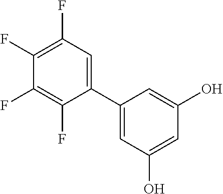 [SMILES: Oc1cc(O)cc(-c2cc(F)c(F)c(F)c2F)c1] |

For Inventive Example 12, 3,5-difluoro-phenylboronic acid (16.08 g), 1-bromo-3,5-dimethoxy-benzene (17 g), potassium carbonate (19.5 g), Pd(PPh₃)₄ (0.45 g), water (68 g), and toluene (206 g) were stirred and refluxed under nitrogen for 4 hours. The resulting toluene solution was separated and dried over MgSO₄. Toluene was removed by distillation and the resulting (MeO)₂C₆H₃—C₆F₂H₃ was distilled under vacuum (141-145° C./1.3 Torr). The obtained (MeO)₂C₆H₃—C₆F₂H₃ (11.8 g) was reacted with 48% hydrobromic acid (39 g) and acetic acid (34.6 g), at a temperature 114° C. for 20 hours. A majority of the acids were removed by distillation in vacuum, and the distillation residue was neutralized with 6.9 g of 25% aqueous NaOH solution, extracted with ethyl acetate and dried over MgSO₄. Ethyl acetate was removed by distillation, and the resulting 3,5-(HO)₂C₆H₃—C₆F₂H₃ was recrystallized to obtain a final off-white solid (7.55 g, m.p.=144° C.) of Inventive Example 12.

For Inventive Example 13, a 250 mL reactor was charged with potassium carbonate (17.7 g), N,N-dimethylformamide (DMF, 65.56 g), and pentafluorobenzene (10.6 g) and pre-heated to 80° C. A mixture of 3,5-dimethoxyphenol (9.8 g) and DMF (11.9 g) was added over 20 min from a dropping funnel. The mixture was stirred and heated to 90° C. for 8 h. Then, additional 1.9 g of pentafluorobenzene was added and the heating continued for 9 hours to achieve 98% conversion of 3,5-dimethoxyphenol by GC/MS. Water (150 g) was added, and crude 3,5-dimethoxyphenyl 2,3,5,6-tetrafluorophenyl ether was filtered as a solid and purified by distillation in vacuum at 102-118° C./0.6-0.8 Torr. Then 3,5-dimethoxyphenyl 2,3,5,6-tetrafluorophenyl ether (MeO)₂C₆H₃—O—C₆F₄H (12.9 g) was diluted in dichloromethane (68 g), and a 1M BBr₃ solution in dichloromethane (60 mL) was added at −14 to −3° C. over 15 min and allowed to stir and slowly warm to room temperature overnight. The mixture was cooled with an ice-water bath, and water was carefully added dropwise. The product was extracted with ethyl acetate. The extract was dried and filtered and the solvent was evaporated to obtain a final yellow powder (11 g, m.p.=142° C.) of Inventive Example 13.

For Inventive Example 14, 85 g (0.34 mol) of boron tribromide was added dropwise to a vigorously stirred solution of 25 g (0.16 mol) of 3,5-dimethoxy-1-fluorobenzene in 200 mL of dichloromethane, at 0-3° C. (ice-water cooling bath). Stirring of the reaction mixture continued while warming up to room temperature and then the mixture was left to stir overnight at room temperature. The reaction mixture was then cooled with ice-water and quenched with deionized water. Solvent was removed with a rotary evaporator, and the crude product was extracted from the mixture with diethyl ether. The crude product was purified by passing through a silica gel column. This synthesis was repeated using the same loads of the starting materials, and the product from the two batches was combined. The combined material was recrystallized from toluene and dried to remove residual solvent to yield 27.5 g (67% average of the two batches) of Inventive Example 14.

For Inventive Example 28, a solution of 3,5-dimethoxy-phenyl magnesium bromide in 2-methyl tetrahydrofuran (32 mL, 0.71 mol) was added over 5 min to a flask containing pentafluoropyridine (9.5 g, 0.56 mol) at 10° C. After reacting at room temperature for 40 min, the reaction was heated to 52° C. for 24 h. The reaction was then quenched with water, washed, and dried over MgSO₄. Solvent removed was removed on a rotary evaporator under vacuum to obtain the crude adduct (13.1 g) which, according to ¹H and ¹⁹F NMR, contained 4-(3,5-dimethoxyphenyl)-2,3,5,6-tetrafluoropyridine and 2-(3,5-dimethoxyphenyl)-3,4,5,6-tetrafluoropyridine (64.5: 35.5 ratio by GC/MS). After two re-crystallizations from toluene, a white crystalline solid was obtained containing 4-(3,5-dimethoxyphenyl)-2,3,5,6-tetrafluoropyridine and 2-(3,5-dimethoxyphenyl)-3,4,5,6-tetrafluoropyridine (99:1 by GC/MS and ¹⁹F NMR). 5.6 g of re-crystallized product was reacted with 48% hydrobromic acid (19 g) and acetic acid (27 g), at 114-116° C. for 12 h. After confirmation of the complete conversion by GC/MS, the majority of excess acids was removed by distillation in vacuum, and the distillation residue was diluted with ethyl acetate and neutralized with 6.4 g of 25% aqueous NaOH solution. The organic layer was washed with water and dried over MgSO₄. The ethyl acetate was removed by rotary evaporation and vacuum drying to obtain 4.6 g (m.p.=214-218° C.) of Inventive Example 28 (5-(2,3,5,6-tetrafluoropyridin-4-yl)benzene-1,3-diol) as a white solid.

The melting points of the inventive examples were determined, except for IE20, and are given in Table 10.

TABLE 10
Melting Points of Inventive Examples

| Inventive Example | Melting Point (° C.) |
|---|---|
| 1 | 65 |
| 2 | 187 |
| 3 | 105 |
| 4 | 119 |
| 5 | 161 |
| 6 | 207 |
| 7 | 112 |
| 8 | 185 |
| 9 | 144 |
| 10 | 175-184 |
| 11 | 87 |
| 12 | 144 |
| 13 | 142 |
| 14 | 135 |
| 15 | 161 |
| 16 | 124-127 |
| 17 | 140 |
| 18 | 144 |
| 19 | 131-134 |
| 20 | N/D |
| 21 | 94 |
| 22 | 117 |
| 23 | 229-230 |
| 24 | 194-195 |
| 25 | 217 |
| 26 | 97-99 |
| 27 | 135 |
| 28 | 214-218 |
| 29 | 146-147 |

### Comparative Fluoroelastomer Curing Examples

Since conditions for each set of curing runs was slightly different, 2,2-bis(4-hydroxyphenyl)hexafluoropropane (BPAF) (Comparative Examples A-P) was used as the curing agent for comparison for each set of curing runs for Inventive Examples of curing agents.

Comparative fluoroelastomer curing compositions included 100 parts by weight Viton™ A-500 (The Chemours Company FC LLC, Wilmington, DE) as the polyhydroxy-curable fluoroelastomer, 30 parts by weight medium thermal carbon black (MT Black) as a filler, 3 parts by weight powdered MgO (Elastomag® 170, Akrochem Corporation, Akron, OH) as an acid acceptor, 6 part by weight calcium hydroxide (Hallstar International, Chicago, IL) as an acid acceptor, 2 parts by weight BPAF, and 0.55 parts by weight benzyl triphenyl phosphonium chloride (BTPPC) as a curing accelerator. The only exception was Comparative Example B, which included 3.3 parts by weight powdered MgO instead of 3.

In an additional comparative example, 2,3,5,6-tetrafluorohydroquinone, obtained from Synquest Laboratories, Inc. (Alachua, FL), was tested as a polyfluorinated hydroquinone curing agent with fluorine atoms at the R₁, R₂, R₄, and R₅ sites. The curing response was extremely low, achieving only very low M_{H} values of 3.77 and 5.36 dN·m, which was too low of a cure state to mold and measure the physical properties.

### Inventive Fluoroelastomer Curing Examples

The prepared fluorinated resorcinol and hydroquinone analogs were evaluated as curing agents (Inventive Examples 1-29) in curing compositions.

Inventive fluoroelastomer curing compositions included 100 parts by weight Viton™ A-500 as the polyhydroxy-curable fluoroelastomer, 30 parts by weight MT Black as a filler, 3 parts by weight powdered MgO as an acid acceptor, 6 part by weight calcium hydroxide as an acid acceptor, 1.02 to 2.42 parts by weight curing agent, and 0.25 to 0.60 parts by weight BTPPC as a curing accelerator. The amounts of curing agent and BTPPC for each curing composition are shown in Table 11. For Inventive Example 20, the curing agent was provided in the form of 10% by weight on MT Black in a total amount of 19.33 parts by weight.

TABLE 11
Fluoroelastomer Curing Compositions

| Inventive Example | Amount | BTPPC |
|---|---|---|
| 1 | 1.30 | 0.25 |
| 2 | 1.85 | 0.35 |
| 3 | 2.04 | 0.45 |
| 4 | 2.00 | 0.25 |
| 5 | 1.60 | 0.55 |
| 6 | 2.08 | 0.39 |
| 7 | 1.63 | 0.33 |
| 8 | 1.65 | 0.45 |
| 9 | 1.75 | 0.48 |
| 10 | 1.71 | 0.41 |
| 11 | 1.74 | 0.38 |
| 12 | 1.63 | 0.50 |
| 13 | 1.89 | 0.37 |
| 14 | 1.02 | 0.42 |
| 15 | 1.63 | 0.33 |
| 16 | 1.87 | 0.41 |
| 17 | 2.06 | 0.48 |
| 18 | 1.96 | 0.60 |
| 19 | 1.53 | 0.33 |
| 20 | 1.93 | 0.33 |
| 21 | 1.91 | 0.42 |
| 22 | 2.42 | 0.39 |
| 23 | 1.76 | 0.35 |
| 24 | 1.67 | 0.44 |
| 25 | 1.60 | 0.40 |
| 26 | 2.04 | 0.43 |
| 27 | 1.42 | 0.40 |
| 28 | 2.12 | 0.60 |
| 29 | 1.68 | 0.36 |

In some cases, several runs were made with the same curing agent, where the amount of curing agent and BTPPC were adjusted based on previous results to obtain cure properties and/or fluoroelastomer properties more similar to those with BPAF as the curing agent.

### Fluoroelastomer Curing Results

Cure properties for the Inventive Examples of Table 9 in the curing compositions of Table 11 and their respective Comparative Examples are shown in Tables 12-15. Each Comparative Example is listed directly before the Inventive Example(s) from the same set of MDR runs. When multiple runs were made with the same curing agent, only the run with the best combination of curing and fluoroelastomer properties was selected for inclusion in Tables 12-15.

TABLE 12
Cure Properties (Comparative Examples A-E and Inventive Examples 1-6)

| Example | A | 1 | B | 2 | C | 3 | D | 4 | E | 5 | 6 |
|---|---|---|---|---|---|---|---|---|---|---|---|
| M_{L} (dN · m) | 1.27 | 1.59 | 1.27 | 1.12 | 1.28 | 1.00 | 1.28 | 1.03 | 0.90 | 0.87 | 0.76 |
| M_{H} (dN · m) | 25.87 | 24.89 | 24.61 | 26.30 | 24.10 | 25.81 | 23.66 | 22.04 | 24.18 | 25.11 | 24.69 |
| ts1 (min) | 1.35 | 1.30 | 1.35 | 1.46 | 1.45 | 1.25 | 1.33 | 1.18 | 1.07 | 0.97 | 1.07 |
| ts2 (min) | 1.55 | 1.71 | 1.53 | 1.69 | 1.66 | 1.43 | 1.52 | 1.45 | 1.20 | 1.09 | 1.23 |
| t₅₀ (min) | 1.97 | 3.48 | 1.95 | 2.34 | 2.14 | 2.02 | 1.91 | 2.53 | 1.51 | 1.42 | 1.77 |
| t₉₀ (min) | 3.18 | 6.61 | 2.70 | 3.14 | 3.07 | 2.93 | 2.60 | 3.69 | 2.21 | 1.92 | 2.44 |

TABLE 13
Cure Properties (Comparative Examples F-H and Inventive Examples 7-14)

| Example | F | 7 | 8 | 9 | G | 10 | 11 | 12 | H | 13 | 14 |
|---|---|---|---|---|---|---|---|---|---|---|---|
| M_{L} (dN · m) | 0.93 | 0.89 | 0.91 | 0.90 | 0.99 | 0.85 | 0.71 | 0.87 | 0.95 | 0.82 | 1.04 |
| M_{H} (dN · m) | 23.39 | 21.05 | 24.78 | 24.99 | 24.58 | 24.20 | 23.63 | 24.99 | 24.02 | 23.96 | 23.11 |
| ts1 (min) | 1.14 | 0.99 | 0.98 | 1.03 | 1.03 | 1.04 | 1.07 | 1.06 | 1.09 | 1.08 | 0.97 |
| ts2 (min) | 1.27 | 1.16 | 1.11 | 1.18 | 1.15 | 1.19 | 1.25 | 1.19 | 1.22 | 1.26 | 1.15 |
| t₅₀ (min) | 1.58 | 1.68 | 1.46 | 1.59 | 1.46 | 1.64 | 1.81 | 1.55 | 1.53 | 1.85 | 1.77 |
| t₉₀ (min) | 2.21 | 2.41 | 2.03 | 2.24 | 2.15 | 2.28 | 2.70 | 2.14 | 2.20 | 2.68 | 2.68 |

TABLE 14
Cure Properties (Comparative Examples I-L and Inventive Examples 15-22)

| Example | I | 15 | J | 16 | 17 | K | 18 | 19 | 20 | L | 21 | 22 |
|---|---|---|---|---|---|---|---|---|---|---|---|---|
| M_{L} (dN · m) | 0.91 | 0.77 | 0.96 | 0.72 | 0.57 | 0.93 | 0.91 | 0.92 | 1.17 | 0.91 | 0.83 | 0.65 |
| M_{H} (dN · m) | 23.63 | 20.66 | 24.59 | 24.20 | 22.54 | 23.63 | 24.65 | 19.73 | 20.30 | 23.84 | 25.14 | 22.29 |
| ts1 (min) | 1.08 | 0.95 | 1.24 | 1.02 | 1.01 | 1.16 | 1.56 | 1.32 | 0.54 | 1.13 | 0.89 | 1.03 |
| ts2 (min) | 1.20 | 1.10 | 1.41 | 1.17 | 1.16 | 1.29 | 1.90 | 1.58 | 0.63 | 1.27 | 1.01 | 1.22 |
| t₅₀ (min) | 1.48 | 1.55 | 1.79 | 1.63 | 1.60 | 1.62 | 2.58 | 2.26 | 1.10 | 1.58 | 1.40 | 2.09 |
| t₉₀ (min) | 2.06 | 2.27 | 2.62 | 2.32 | 2.40 | 2.34 | 3.64 | 3.34 | 2.25 | 2.20 | 1.91 | 3.28 |

TABLE 15
Cure Properties (Comparative Examples M-P and Inventive Examples 23-29)

| Example | M | 23 | 24 | N | 25 | O | 26 | 27 | 28 | P | 29 |
|---|---|---|---|---|---|---|---|---|---|---|---|
| M_{L} (dN · m) | 0.83 | 0.75 | 0.84 | 1.05 | 1.02 | 1.17 | 1.16 | 1.35 | 1.03 | 1.18 | 1.18 |
| M_{H} (dN · m) | 23.13 | 23.25 | 23.89 | 24.44 | 21.86 | 26.10 | 25.57 | 23.05 | 26.09 | 26.61 | 25.11 |
| ts1 (min) | 1.10 | 1.01 | 0.99 | 1.28 | 1.25 | 1.23 | 1.06 | 1.48 | 0.97 | 1.16 | 1.03 |
| ts2 (min) | 1.24 | 1.19 | 1.16 | 1.48 | 1.56 | 1.39 | 1.22 | 1.79 | 1.12 | 1.31 | 1.20 |
| t₅₀ (min) | 1.57 | 1.77 | 1.64 | 1.94 | 2.41 | 1.76 | 1.80 | 2.60 | 1.63 | 1.66 | 1.79 |
| t₉₀ (min) | 2.52 | 2.99 | 2.58 | 3.16 | 3.95 | 2.70 | 2.95 | 3.99 | 2.46 | 2.42 | 2.65 |

Tables 12-15 show that the Inventive Examples provided cure properties that were similar to the cure properties of BPAF. Tables 12-15 show that for the Inventive Examples, M_{L} values for the were in the range of 0.57 to 1.59 dN·m, M_{H} values were in the range of 19.73 to 26.3 dN·m, ts1 values were in the range of 0.54 to 1.56 minutes, ts2 values were in the range of 0.63 to 1.90 minutes, t₅₀ values were in the range of 1.10 to 3.48 minutes, and t₉₀ values were in the range of 1.91 to 6.61 minutes.

### Fluoroelastomer Properties

Fluoroelastomer properties for the fluoroelastomers formed from the Inventive Examples of Table 9 in the curing compositions of Table 11 and the fluoroelastomers formed from their respective Comparative Examples are shown in Tables 16-19. When multiple runs were made with the same curing agent, only the run with the best combination of curing and fluoroelastomer properties was selected for inclusion in Tables 16-19.

TABLE 16
Fluoroelastomer Properties (Comparative Examples A-E and Inventive Examples 1-6)

| Example | A | 1 | B | 2 | C | 3 | D | 4 | E | 5 | 6 |
|---|---|---|---|---|---|---|---|---|---|---|---|
| TS [MPa] | 15.7 | 15.5 | 16.1 | 15.0 | 15.2 | 15.4 | 14.5 | 15.2 | 13.7 | 15.7 | 15.6 |
| EB (%) | 212 | 174 | 206 | 168 | 213 | 181 | 212 | 204 | 181 | 187 | 149 |
| M100 [MPa] | 6.7 | 7.5 | 6.6 | 7.4 | 6.2 | 7.3 | 6.0 | 5.6 | 6.3 | 7.0 | 8.7 |
| CS1 (%) | 19.1 | 16.9 | 21.3 | 19.1 | 18.0 | 18.2 | 20.2 | 15.7 | 18.6 | 25.6 | 18.6 |
| CS2 (%) | 30.3 | 23.6 | 33.7 | 29.2 | 30.7 | 32.6 | 30.7 | 25.8 | 31.4 | 34.5 | 29.1 |
| CS3 (%) | N/D | N/D | N/D | N/D | N/D | N/D | N/D | N/D | 66.3 | 68.6 | 59.3 |

TABLE 17
Fluoroelastomer Properties (Comparative Examples F-H and Inventive Examples 7-14)

| Example | F | 7 | 8 | 9 | G | 10 | 11 | 12 | H | 13 | 14 |
|---|---|---|---|---|---|---|---|---|---|---|---|
| TS [MPa] | 15.3 | 15.2 | 15.7 | 14.3 | 13.4 | 15.4 | 13.5 | 15.0 | 12.8 | 15.1 | 16.5 |
| EB [%] | 207 | 212 | 190 | 167 | 186 | 183 | 182 | 187 | 176 | 172 | 170 |
| M100 [MPa] | 5.9 | 5.3 | 6.8 | 7.3 | 6.1 | 6.9 | 6.3 | 6.8 | 3.0 | 3.1 | 3.3 |
| CS1 (%) | 18.4 | 19.5 | 20.9 | 20.9 | 18.4 | 19.8 | 20.7 | 20.7 | 19.8 | 20.9 | 19.8 |
| CS2 (%) | 30.2 | 31.0 | 31.4 | 32.6 | 27.9 | 27.9 | 30.2 | 31.8 | 27.6 | 34.9 | 32.6 |
| CS3 (%) | 60.5 | 58.6 | 62.8 | 65.5 | 62.4 | 59.3 | 62.8 | 63.2 | 59.3 | 62.8 | 80.2 |

TABLE 18
Fluoroelastomer Properties (Comparative Examples I-L and Inventive Examples 15-22)

| Example | I | 15 | J | 16 | 17 | K | 18 | 19 | 20 | L | 21 | 22 |
|---|---|---|---|---|---|---|---|---|---|---|---|---|
| TS [MPa] | 14.1 | 14.2 | 15.7 | 15.8 | 16.1 | 14.8 | 13.8 | 16.0 | 17.1 | 15.2 | 17.1 | 13.3 |
| EB [%] | 202 | 188 | 205 | 190 | 160 | 197 | 180 | 215 | 215 | 197 | 185 | 135 |
| M100 [MPa] | 5.9 | 5.6 | 6.1 | 6.4 | 8.0 | 6.2 | 7.2 | 5.5 | 5.6 | 6.3 | 7.6 | 8.1 |
| CS1 (%) | 16.3 | 18.4 | 16.3 | 20.9 | 22.1 | 17.4 | 23.3 | 20.0 | 18.6 | 18.6 | 19.8 | 25.6 |
| CS2 (%) | 28.7 | 31 | 25.6 | 31.4 | 33.7 | 27.9 | 36.5 | 28.4 | 32.6 | 30.2 | 31.0 | 40.7 |
| CS3 (%) | 60.9 | 60.9 | 60.5 | 65.1 | 68.6 | 62.8 | 71.6 | 62.4 | 73.3 | 62.8 | 63.2 | 75.6 |

TABLE 19
Fluoroelastomer Properties (Comparative Examples
M-P and Inventive Examples 23-29)

| Example | M | 23 | 24 | N | 25 | O | 26 | 27 | 28 | P | 29 |
|---|---|---|---|---|---|---|---|---|---|---|---|
| TS [MPa] | 13.9 | 15.8 | 15.2 | 13.5 | 13.2 | 14.0 | 14.6 | 15.2 | 14.1 | 14.5 | 13.7 |
| EB [%] | 212 | 200 | 208 | 227 | 195 | 182 | 170 | 195 | 144 | 190 | 170 |
| M100 [MPa] | 5.1 | 6.0 | 5.9 | 5.2 | 5.7 | 6.5 | 7.3 | 6.4 | 8.0 | 6.9 | 7.1 |
| CS1 (%) | 17.4 | 17.4 | 19.5 | 16.3 | 19.8 | 17.2 | 19.5 | 20.7 | 20.7 | 22.7 | 21.8 |
| CS2 (%) | 26.7 | 25.6 | 30.2 | 25.6 | 29.4 | 26.4 | 27.6 | 33.0 | 32.2 | 29.5 | 31.0 |
| CS3 (%) | 62.8 | 55.8 | 61.6 | 62.8 | 61.6 | 65.5 | 65.1 | 69.3 | 64.4 | 67.0 | 63.2 |

Tables 16-19 show that the Inventive Examples provided a cured fluoroelastomer having similar properties to a cured fluoroelastomer formed with BPAF as the curing agent. Tables 16-19 show that for the Inventive Examples, TS values for the were in the range of 13.2 to 17.1 MPa, EB values were in the range of 135 to 215%, M100 values were in the range of 3.06 to 8.69 MPa, CS1 values were in the range of 15.7 to 25.6%, CS2 values were in the range of 23.6 to 40.7%, and CS3 values were in the range of 55.8 to 80.2%.

Finally, Inventive Example 1 was fluid aged and then tested. The swell of the fluid-aged fluoroelastomer formed with Inventive Example 1 as the curing agent was 3.5 wt % with a standard deviation of 0.2 wt %, and the CS1 of the fluid-aged In fluoroelastomer formed with Inventive Example 1 as the curing agent was 22.0%. These results indicate that the acid resistance was excellent for fluoroelastomer formed with fluorinated resorcinol and hydroquinone analogs as the curing agent.

### Curing Agent Salt Examples

### Curing Agent Salt Formation

To form an inventive example of a curing agent salt, Inventive Example 9 (IE9) was pre-reacted with BTPPC by the following procedure. A 500-mL round bottom flask equipped with a magnetic stir bar and dropping addition funnel topped with a nitrogen-tee was charged with IE9 (20.34 g, 0.0847 mol) and methanol (45 g). 25 wt % sodium methylate (18.53 g, 0.0857 mol) in methanol was then added rapidly via the addition funnel and the solution was stirred at room temperature for 15 min. Next a solution of BTPPC (33.0 g, 0.0849 mol) in methanol (17.9 g) was added rapidly via the addition funnel. The mixture was stirred for 30 min while sodium chloride precipitated. The slurry was filtered through a polypropylene filter funnel with a 10-micron polyethylene frit to remove sodium chloride, then the filtrate, which contained BTPP⁺IE9⁻ salt, was combined with a 3.5-fold molar excess of IE9 (71.34 g, 0.297 mol) dissolved in methanol (200 mL). The bulk of the methanol was removed with a rotary evaporator, then the residual methanol was removed under vacuum with magnetic stirring at 150° C./30 torr. While the mixture was still molten, a portion was removed and rapidly cooled with liquid nitrogen then dried under vacuum at room temperature for 18 hours. The solid designated as IE9/PRC1 (65.2 g) was recovered and determined by ¹H NMR in MeOH-d₄ to be 4.50:1 IE9:BTPP+ mol:mol. The balance of the molten mixture was allowed to cool to room temperature then was dried in an 80° C. vacuum oven for 18 hours. The solid designated as IE9/PRC2 (53.0 g) was recovered and determined by ¹H NMR in MeOH-d₄ to be 4.49:1 IE9:BTPP+ mole ratio. The overall yield was 98.7%. The structure of the BTPP⁺IE9⁻ salt is shown as Formula 4.

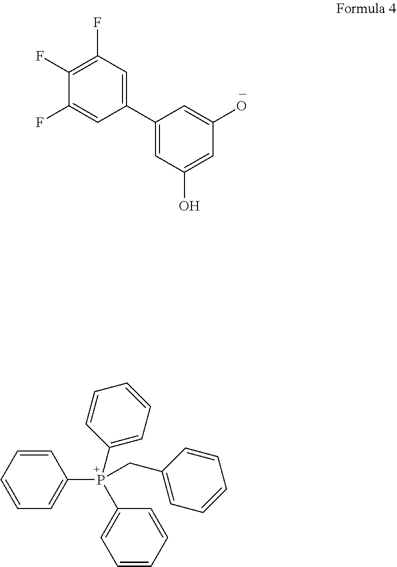

In a similar manner, a BTPP⁺IE9⁻ salt was formed using a 3-fold molar excess of IE9. The product isolated by the rapid cooling method was designated IE9/PRC3 and was determined by ¹H NMR in MeOH-d₄ to be 4.07:1 IE9:BTPP+ mol:mol.

In a similar manner, a BTPP⁺IE9⁻ salt was formed using a 4-fold molar excess of IE9. The product isolated by the rapid cooling method was designated IE9/PRC4 and was determined by ¹H NMR in MeOH-d₄ to be 5.08:1 IE9:BTPP+ mol:mol.

To form an inventive example of a curing agent salt, Inventive Example 23 (IE23) was pre-reacted with BTPPC by a procedure as for IE9 to form a BTPP⁺IE9⁻ salt using a 3-fold molar excess of IE23. While the product mixture was still molten, a portion was removed and rapidly cooled with liquid nitrogen then dried under vacuum at room temperature for 18 hours. The solid, designated as IE23/PRC1 (5.09 g), was recovered and determined by ¹H NMR in MeOH-d₄ to be 4.05:1 I-23:BTPP+ mol:mol. The balance of the molten mixture was allowed to cool to room temperature then was dried in an 80° C. vacuum oven for 18 hours. The solid designated as IE23/PRC2 (6.56 g) was recovered and determined by ¹H NMR in MeOH-d₄ to be 4.00:1 IE23:BTPP+ mol:mol. The overall yield was 100%.

For comparison, Viton™ VC-50, a pre-reacted salt blend of BPAF and BTPP+ available from Chemours, was used.

### Curing Examples with Curing Agent Salts

Comparative and inventive fluoroelastomer curing compositions included 100 parts by weight Viton™ B-600 as the polyhydroxy-curable fluoroelastomer, 30 parts by weight MT Black as a filler, 3 parts by weight powdered MgO as an acid acceptor, 6 parts by weight calcium hydroxide as an acid acceptor, and equimolar amounts of BPAF and IE9, either not pre-reacted or pre-reacted with BTPPC. Viton™ B-600 fluoroelastomer is a terpolymer of hexafluoropropylene, vinylidene fluoride, and tetrafluoroethylene available from Chemours. The amount of pre-reacted salt was adjusted for the curatives to cure to t₉₀ in less than 3 min. IE9, for example, required slightly less salt to do this. Curings were then compared at the fixed levels of BPAF and IE9 or IE23. For the cases of IE9/PRC1 and IE9/PRC2, for example, since IE9 cures with less BTPP+, an additional 0.35 phr IE9 was added to achieve equimolar loading of IE9.

Cure properties for curing compositions with curing agents that were not pre-reacted or with pre-reacted curing agents are shown in Table 20. In the first set of MDR runs, the curing agents were BPAF (BPAF1), pre-reacted BPAF (BPAFS), IE9 (IE91), IE9/PRC1 (9/1), and IE9/PRC2 (9/2). In the second set of MDR runs, the curing agents were BPAF (BPAF2), IE9 (IE92), IE9/PRC3 (9/3), and IE9/PRC4 (9/4). In the third set of MDR runs, the curing agents were BPAF (BPAF3), IE23 (IE23), IE23/PRC1 (23/1), and IE23/PRC2 (23/2).

TABLE 20
Cure Properties

| Example | BPAF1 | BPAFS | IE91 | 9/1 | 9/2 | BPAF2 | IE92 | 9/3 | 9/4 | BPAF3 | IE23 | 23/1 | 23/2 |
|---|---|---|---|---|---|---|---|---|---|---|---|---|---|
| M_{L} (dN · m) | 1.08 | 0.94 | 0.81 | 0.87 | 0.90 | 1.14 | 1.06 | 1.14 | 1.17 | 1.38 | 1.31 | 1.33 | 1.41 |
| M_{H} (dN · m) | 26.25 | 25.71 | 18.54 | 21.63 | 21.89 | 22.54 | 20.25 | 22.83 | 23.36 | 21.85 | 20.42 | 21.12 | 17.18 |
| ts1 (min) | 1.01 | 1.49 | 0.83 | 1.00 | 1.02 | 1.21 | 1.01 | 1.06 | 1.09 | 2.28 | 1.52 | 1.49 | 2.21 |
| ts2 (min) | 1.12 | 1.70 | 0.95 | 1.17 | 1.21 | 1.37 | 1.20 | 1.26 | 1.30 | 2.73 | 1.87 | 1.97 | 3.01 |
| t₅₀ (min) | 1.40 | 2.11 | 1.29 | 1.65 | 1.70 | 1.70 | 1.84 | 1.82 | 1.92 | 3.55 | 2.73 | 3.14 | 4.75 |
| t₉₀ (min) | 2.00 | 2.92 | 1.75 | 2.28 | 2.34 | 2.31 | 2.60 | 2.45 | 2.59 | 5.14 | 4.01 | 4.70 | 7.26 |

Table 20 shows that both Inventive Examples that were not pre-reacted and pre-reacted Inventive Examples provided cure properties that were similar to the cure properties of BPAF.

### Fluoroelastomer Properties

Fluoroelastomer properties for the fluoroelastomers whose cure properties are shown in Table 20 are shown in Table 21.

TABLE 21
Fluoroelastomer Properties

| Example | BPAF1 | BPAFS | IE91 | 9/1 | 9/2 | BPAF2 | IE92 | 9/3 | 9/4 | BPAF3 | IE23 | 23/1 | 23/2 |
|---|---|---|---|---|---|---|---|---|---|---|---|---|---|
| TS [MPa] | 12.5 | 14.0 | 15.0 | 13.4 | 13.6 | 14.2 | 14.4 | 14.3 | 15.4 | 13.8 | 16.0 | 14.8 | 14.8 |
| EB [%] | 182 | 202 | 251 | 214 | 225 | 213 | 246 | 228 | 263 | 204 | 235 | 216 | 223 |
| M100 [MPa] | 6.1 | 5.9 | 4.8 | 4.7 | 4.8 | 5.4 | 4.9 | 5.1 | 5.2 | 5.4 | 5.2 | 5.6 | 5.3 |
| CS1 (%) | 25.3 | 19.8 | 39.5 | 24.1 | 25.3 | 25.3 | 34.5 | 21.8 | 21.6 | 25.3 | 27.3 | 23.0 | 25.3 |
| ΔCS1 | | 5.5 | | 15.4 | 14.2 | | | 12.7 | 12.9 | | | 4.3 | 2.0 |
| CS2 (%) | 33.3 | 5.4 | 50.6 | 34.5 | 33.3 | 36.8 | 46.0 | 32.2 | 31.8 | 34.9 | 37.9 | 32.2 | 40.2 |
| ΔCS2 | | | | 16.1 | 17.3 | | | 13.8 | 14.2 | | | 5.7 | −2.3 |
| CS3 (%) | 74.4 | 76.7 | 82.8 | 72.4 | 72.1 | 75.6 | 77.0 | 69.0 | 69.0 | 72.1 | 67.0 | 60.9 | 65.5 |
| ΔCS3 | | −2.3 | | 10.4 | 10.7 | | | 8.0 | 8.0 | | | 6.1 | 1.5 |

Table 21 shows that the fluoroelastomer properties of fluoroelastomers formed with IE9/PRC1 and IE9/PRC2 were substantially the same. For the fluoroelastomers formed in the first set of MDR runs, the pre-reacted inventive curing agents showed a significantly greater improvement compared to BPAF in CS1 (ΔCS1), CS2 (ΔCS1), and CS3 (ΔCS1) relative to their equivalent that was not pre-reacted. A positive ΔCS value indicates a decrease in CS. The improvement in CS that the pre-reacted IE9 catalyst salt offers was surprisingly significantly larger than for BPAF. Additionally, the pre-reacted BPAF CS was actually worse at 250° C. (CS3), whereas the pre-reacted IE9 CS was improved by 10.4-10.7 points.

The second set of MDR runs, where the IE9:BTPPC molar ratios were slightly lower or slightly higher than in the first set of MDR runs, showed a similar trend with ΔCS values, although the improvements were not quite as good but still substantial.

For the third set of MDR runs, IE23/PRC1 showed a significant but lesser improvement in ΔCS values than IE9. Interestingly, the cure rate and compression set for IE23/PRC1 were superior to IE23/PRC2, which did not show much of an improvement and was actually slightly worse at 168 hours at 200° C. (CS2). This demonstrates that in some instances it is advantageous to cool the melted eutectic catalyst mixture rapidly and dry and store at room temperature prior to rubber compounding rather than slowly cooling only to room temperature prior to drying at 80° C.

All above-mentioned references are hereby incorporated by reference herein.

While the invention has been described with reference to a preferred embodiment, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A curable fluoroelastomer composition comprising:
a polyhydroxy-curable fluoroelastomer;
a curing agent of Formula 1: 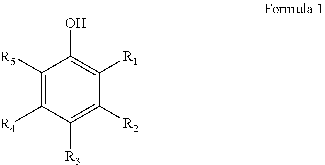
wherein R₁ and R₅ are independently selected from the group consisting of H, halogen, C₁₋₁₈ alkyl which may be partially or fully halogenated, C₁₋₁₈ alkoxy which may be partially or fully halogenated, and X;
wherein R₂, R₃, and R₄ are independently selected from the group consisting of OH, H, halogen, C₁₋₁₈ alkyl which may be partially or fully halogenated, C₁₋₁₈ alkoxy which may be partially or fully halogenated, and X,
with the proviso that at least one of R₂, R₃, and R₄ is OH;
with the proviso that not more than 3 of R₁, R₂, R₃, R₄, and R₅ are halogen;
wherein X is selected from the group consisting of Formula 2 and Formula 3: 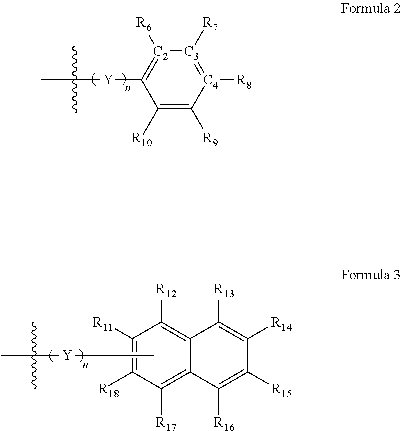
wherein R₆, R₇, R₈, R₉, and R₁₀ are independently selected from the group consisting of H, C₁₋₁₈ alkyl which may be partially or fully halogenated, C₁₋₁₈ alkoxy which may be partially or fully halogenated, partially or fully fluorinated phenyl, acetyl or methylsulfonyl which may be alkyl- or aryl-substituted or partially or fully halogenated, nitro, nitrile, and halogen, with the proviso that N may be substituted at exactly one of C₂, C₃, or C₄ in which case respective R₆, R₇, or R₈ is absent;
wherein R₁₁, R₁₂, R₁₃, R₁₄, R₁₅, R₁₆, R₁₇, and R₁₈ are independently selected from the group consisting of H, C₁₋₁₈ alkyl which may be partially or fully halogenated, C₁₋₁₈ alkoxy which may be partially or fully halogenated, nitro, nitrile, and halogen with the proviso that one of R₁₁, R₁₂, R₁₃, R₁₄, R₁₅, R₁₆, R₁₇, and R₁₈ is a single bond to —(Y)ₙ—;
wherein Y is selected from the group consisting of —SO₂—, —C(O)—, —C(CF₃)₂—, and —O—; and
wherein n is 0 or 1;
with the proviso that at least one of R₁, R₂, R₃, R₄, and R₅, is fluorine, a fluorine-containing C₁₋₁₈ alkyl, a fluorine-containing C₁₋₁₈ alkoxy, or X;
with the proviso that, when X is present and none of R₁, R₂, R₃, R₄, and R₅ is fluorine, a fluorine-containing C₁₋₁₈ alkyl, or a fluorine-containing C₁₋₁₈ alkoxy, at least one of R₆, R₇, R₈, R₉, R₁₀, R₁₁, R₁₂, R₁₃, R₁₄, R₁₅, R₁₆, R₁₇, and R₁₈ is fluorine, a fluorine-containing C₁₋₁₈ alkyl, or a fluorine-containing C₁₋₁₈ alkoxy; and
an acid acceptor.

2. The curable fluoroelastomer composition of claim 1, wherein at least one of R₁ and R₅ is H.

3. The curable fluoroelastomer composition of claim 1, wherein at least one of R₁, R₂, R₃, R₄, and R₅ is H when it is an adjacent substituent to OH.

4. The curable fluoroelastomer composition of claim 1, wherein no more than one of R₁, R₂, R₃, R₄, and R₅ is X.

5. The curable fluoroelastomer composition of claim 1, wherein no more than 2 of R₁, R₂, R₃, R₄, and R₅ are halogen.

6. The curable fluoroelastomer composition of claim 1, wherein only one of R₂, R₃, and R₄ is OH.

7. The curable fluoroelastomer composition of claim 6, wherein either R₂ or R₄ is OH.

8. The curable fluoroelastomer composition of claim 1, wherein R₁ and R₅ are selected from the group consisting of H, fluorine, C₁₋₁₈ alkyl which may be partially or fully fluorinated, C₁₋₁₈ alkoxy which may be partially or fully fluorinated, and X; and wherein R₂, R₃, and R₄ are independently selected from the group consisting of OH, H, fluorine, C₁₋₁₈ alkyl which may be partially or fully fluorinated, C₁₋₁₈ alkoxy which may be partially or fully fluorinated, and X.

9. The curable fluoroelastomer composition of claim 1, wherein one of R₁, R₂, R₃, R₄, and R₅ is X, and X is Formula 2.

10. The curable fluoroelastomer composition of claim 9, wherein R₆, R₇, R₈, R₉, and R₁₀ are independently selected from the group consisting of H, fluorine, C₁₋₆ alkyl which may be partially or fully fluorinated, and C₁₋₆ alkoxy which may be partially or fully fluorinated, and at least one of R₆, R₇, R₈, R₉, and R₁₀ is fluorine, C₁₋₆ alkyl which may be partially or fully fluorinated, or C₁₋₆ alkoxy which may be partially or fully fluorinated.

11. The curable fluoroelastomer composition of claim 9, wherein R₆, R₇, R₈, R₉, and R₁₀ are independently selected from the group consisting of H, fluorine, perfluoromethyl, and perfluoromethoxy, and at least one of R₆, R₇, R₈, R₉, and R₁₀ is fluorine, perfluoromethyl, or perfluoromethoxy.

12. The curable fluoroelastomer composition of claim 1, wherein n is 0.

13. The curable fluoroelastomer composition of claim 1, wherein —Y— is —O—.

14. The curable fluoroelastomer composition of claim 1, wherein the curing agent is selected from the group consisting of:

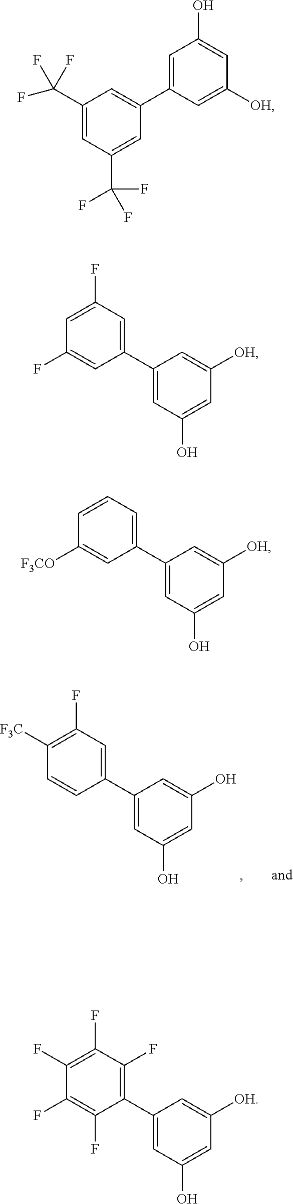 [SMILES: Oc1cc(O)cc(-c2cc(C(F)(F)F)cc(C(F)(F)F)c2)c1 ; Oc1cc(O)cc(-c2cc(F)cc(F)c2)c1 ; Oc1cc(O)cc(-c2cccc(OC(F)(F)F)c2)c1 ; Oc1cc(O)cc(-c2ccc(C(F)(F)F)c(F)c2)c1 ; Oc1cc(O)cc(-c2c(F)c(F)c(F)c(F)c2F)c1]

15. The curable fluoroelastomer composition of claim 1 containing about 0.1 to about 10 parts by weight of said curing agent per 100 parts by weight of said polyhydroxy-curable fluoroelastomer.

16. The curable fluoroelastomer composition of claim 1, wherein the polyhydroxy-curable fluoroelastomer is a copolymer of hexafluoropropylene and vinylidene fluoride.

17. The curable fluoroelastomer composition of claim 1, wherein the polyhydroxy-curable fluoroelastomer is a terpolymer of hexafluoropropylene, vinylidene fluoride, and tetrafluoroethylene.

18. The curable fluoroelastomer composition of claim 1, wherein the acid acceptor is selected from the group consisting of powdered magnesium oxide, calcium hydroxide, and a combination thereof.

19. The curable fluoroelastomer composition of claim 1, wherein the curable fluoroelastomer composition is free of 2,2-bis(4-hydroxyphenyl)hexafluoropropane.

20. The curable fluoroelastomer composition of claim 1 further comprising a curing accelerator.

21. The curable fluoroelastomer composition of claim 20, wherein the curing accelerator is in the form of a pre-reacted salt with a portion of the curing agent.

22. The curable fluoroelastomer composition of claim 21, wherein the curing agent is present in a threefold to fourfold molar excess of the curing accelerator.
